SEQUENCE LISTING (175 sequences)

SEQ ID NO: 1 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 2 - protein, 48 aa, synthetic construct
STEEPVMKKRTYCTYCPSKIRRKANASCKKCKKVICREHNIDMCQSCF

SEQ ID NO: 3 - protein, 656 aa, synthetic construct
MAPKKKRKVGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 4 - DNA, 35 bp, synthetic construct
  source 1..35: mol_type = other DNA
ccctagaaagatagtctgcgtaaaattgacgcatg

SEQ ID NO: 5 - DNA, 63 bp, synthetic construct
  source 1..63: mol_type = other DNA
ccctagaaagataatcatattgtgacgtacgttaaagataatcatgcgtaaaattgacgcatg

SEQ ID NO: 6 - DNA, 35 bp, synthetic construct; identical to SEQ ID NO: 4
  source 1..35: mol_type = other DNA

SEQ ID NO: 7 - DNA, 36 bp, synthetic construct
  source 1..36: mol_type = other DNA
ccctagaaagatagtcatgcgtaaaattgacgcatg

SEQ ID NO: 8 - DNA, 37 bp, synthetic construct
  source 1..37: mol_type = other DNA
ccctagaaagatagtcattgcgtaaaattgacgcatg

SEQ ID NO: 9 - DNA, 38 bp, synthetic construct
  source 1..38: mol_type = other DNA
ccctagaaagatagtcatatgcgtaaaattgacgcatg

SEQ ID NO: 10 - DNA, 38 bp, synthetic construct
  source 1..38: mol_type = other DNA
ccctagaaagataatcatatgcgtaaaattgacgcatg

SEQ ID NO: 11 - DNA, 10 bp, synthetic construct
  source 1..10: mol_type = other DNA
aaagatagtc

SEQ ID NO: 12 - DNA, 10 bp, synthetic construct
  source 1..10: mol_type = other DNA
aaagataatc

SEQ ID NO: 13 - DNA, 8920 bp, synthetic construct
  source 1..8920: mol_type = other DNA
ggcctaactggccggtacctgagtcttccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctcgtccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaaagcttggcaatccggtactgttggtaaagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcaagcttatcgataccgtcgacctcgagggggggcccggtacccaattcgccctatagtgagtcgtattaagatctaattaaccctcactaaagggaacaaaagctggagctccaccgcggtggcggccgctctagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagccataccaatgggccctaaaaaaggaatccagtcaattccggggctaaacctggctgccactgtttctttagggacttcgttcctgtgaggacaggaaaccctgcaacctggttgaccagagcaagggatataagaacccagtttgggaacactgctgtttaaatattaaacagggatgtcttgtcccccggaagggagaccagatgctgcctgggcccccctgagatggtgcctcagcacggcagggctcactgattttttgggggtactgccaacaccttggaattcgcggccaccctcaaaggcatcaccgcgggccaggtgaatatcaaatcctcctcgtttttggaaactgacaatcttagcgcagaagtcatgcccgcttttgagagggagtactcaccccaacagctggcccttgcagatgagaccatggtgcatggcacatgggcgtcgagcggatgtatgctatacgaagttatccgggttgtggcgatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcgagaccatgcatcacacattgatgcgagctgaagcacaagcggctgggtgccggtccataccgctttcttgtgcgcaggacataggcaatgtaccggcggcttgctcttcaacctcaggcggaaagccgtgagaattcgccagaatgcgttcgcacagccgccagccggtcactccgttgatggttactcggaacagcagggagccgtcggggttgatcaggcgctcgtcgataattttgttgccgttccacagggtccctgttacagtgatctttttgccgtcgaacacggcgatgccttcatacggccgtccgaaatagtcgatcatgttcggcgtaaccccgtcgattaccagtgtgccatagtgcaggatcaccttaaagtgatgatcatccacagggtacaccaccttaaaaattttttcgatctggcccatttggtcgccgctcagaccttcatacgggatgatgacatggatgtcgatcttcagcccattttcaccgctcaggacaatcctttggatcggagttacggacaccccgagattctgaaacaaactggacacacctccctgttcaaggacttggtccaggttgtagccggctgtctgtcgccagtccccaacgaaatcttcgagtgtgaagaccatggtggccacacaggcctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccaattcccactcctttcaagacctagaaggtccattagctgcaaagattcctctctgtttaaaactttatccatctttgcaaagctggccttggaggccttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctagaactagtggatcccccgggctgcaggaattcgataaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 14 - protein, 53 aa, synthetic construct
GTSDDSTEEPVMKKRTYCTYCPSKIRRKANASCKKCKKVICREHNIDMCQSCF

SEQ ID NO: 15 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 16 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 17 - protein, 1141 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 18 - protein, 1141 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 19 - protein, 1142 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 20 - protein, 1142 aa, synthetic construct; identical to SEQ ID NO: 19

SEQ ID NO: 21 - DNA, 36 bp, synthetic construct
  source 1..36: mol_type = other DNA
ccctagaaagataatcatgcgtaaaattgacgcatg

SEQ ID NO: 22 - DNA, 1264 bp, synthetic construct
  source 1..1264: mol_type = other DNA
agctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 23 - DNA, 684 bp, synthetic construct
  source 1..684: mol_type = other DNA
atgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccattaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatga

SEQ ID NO: 24 - DNA, 225 bp, synthetic construct
  source 1..225: mol_type = other DNA
gagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagacc

SEQ ID NO: 25 - DNA, 236 bp, synthetic construct
  source 1..236: mol_type = other DNA
atgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccattaa

SEQ ID NO: 26 - DNA, 452 bp, synthetic construct
  source 1..452: mol_type = other DNA
ttaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatga

SEQ ID NO: 27 - protein, 4 aa, synthetic construct
AGGG

SEQ ID NO: 28 - protein, 563 aa, synthetic construct
TLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 29 - protein, 562 aa, synthetic construct
LPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 30 - protein, 561 aa, synthetic construct
PQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 31 - protein, 560 aa, synthetic construct
QRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 32 - protein, 559 aa, synthetic construct
RTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 33 - protein, 558 aa, synthetic construct
TIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 34 - protein, 557 aa, synthetic construct
IRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 35 - protein, 556 aa, synthetic construct
RGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 36 - protein, 555 aa, synthetic construct
GKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 37 - protein, 554 aa, synthetic construct
KNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 38 - protein, 553 aa, synthetic construct
NKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 39 - protein, 552 aa, synthetic construct
KHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 40 - protein, 551 aa, synthetic construct
HCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 41 - protein, 550 aa, synthetic construct
CWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 42 - protein, 549 aa, synthetic construct
WSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 43 - protein, 548 aa, synthetic construct
STSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 44 - protein, 547 aa, synthetic construct
TSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 45 - protein, 546 aa, synthetic construct
SKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 46 - protein, 545 aa, synthetic construct
KSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 47 - protein, 544 aa, synthetic construct
STRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 48 - protein, 543 aa, synthetic construct
TRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 49 - protein, 564 aa, synthetic construct
TLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 50 - protein, 563 aa, synthetic construct
LPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 51 - protein, 562 aa, synthetic construct
PQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 52 - protein, 561 aa, synthetic construct
QRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 53 - protein, 560 aa, synthetic construct
RTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 54 - protein, 559 aa, synthetic construct
TIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 55 - protein, 558 aa, synthetic construct
IRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 56 - protein, 557 aa, synthetic construct
RGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 57 - protein, 556 aa, synthetic construct
GKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 58 - protein, 555 aa, synthetic construct
KNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 59 - protein, 554 aa, synthetic construct
NKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 60 - protein, 553 aa, synthetic construct
KHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 61 - protein, 552 aa, synthetic construct
HCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 62 - protein, 551 aa, synthetic construct
CWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 63 - protein, 550 aa, synthetic construct
WSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 64 - protein, 549 aa, synthetic construct
STSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 65 - protein, 548 aa, synthetic construct
TSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 66 - protein, 547 aa, synthetic construct
SKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 67 - protein, 546 aa, synthetic construct
KSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 68 - protein, 545 aa, synthetic construct
STRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 69 - protein, 544 aa, synthetic construct
TRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 70 - protein, 594 aa, synthetic construct
GGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 71 - protein, 646 aa, synthetic construct
GGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 72 - protein, 699 aa, synthetic construct
GGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 73 - protein, 646 aa, synthetic construct
GGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 74 - protein, 99 aa, synthetic construct
GGGGSERPYACPVESCDRRFSRSDELTRHIRIHTGQKPFQCRICMRNFSRSDHLTTHIRTHTGEKPFACDICGRKFARSDERKRHTKIHLRQKDGGGGS

SEQ ID NO: 75 - protein, 136 aa, synthetic construct
VDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 76 - protein, 66 aa, synthetic construct
SIVAQLSRPDPALAALTNDHLVALACLGGRPALDAVKKGLPHAPALIKRTNRRIPERTSHRVATAL

SEQ ID NO: 77 - protein, 136 aa, synthetic construct
VDLRTLGYSQQQQEKIKPKVRSTVAQHHEALVGHGFTHAHIVALSQHPAALGTVAVKYQDMIAALPEATHEAIVGVGKSRSGARALEALLTVAGELRGPPLQLDTGQLLKIAKRGGVTAVEAVHAWRNALTGAPLN

SEQ ID NO: 78 - protein, 135 aa, synthetic construct
VDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLN

SEQ ID NO: 79 - protein, 34 aa, synthetic construct
  VARIANT 11..13
LTPDQVVAIAXXXGGKQALETVQRLLPVLCQDHG

SEQ ID NO: 80 - protein, 34 aa, synthetic construct
  VARIANT 11..13
LTPEQVVAIAXXXGGKQALETVQRLLPVLCQAHG

SEQ ID NO: 81 - protein, 34 aa, synthetic construct
  VARIANT 11..13
LTPDQVVAIAXXXGGKQALETVQRLLPVLCQAHG

SEQ ID NO: 82 - protein, 34 aa, synthetic construct
  VARIANT 11..13
LTPAQVVAIAXXXGGKQALETVQRLLPVLCQDHG

SEQ ID NO: 83 - protein, 20 aa, synthetic construct
  VARIANT 11..13
LTPEQVVAIAXXXGGRPALE

SEQ ID NO: 84 - protein, 326 aa, synthetic construct
LTPDQVVAIASNIGGKQALETVQRLLPVLCQDHGLTPEQVVAIASHDGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIANNNGGKQALETVQRLLPVLCQDHGLTPEQVVAIASHDGGRPALE

SEQ ID NO: 85 - DNA, 11 bp, synthetic construct
  source 1..11: mol_type = other DNA
tacgcccacgc

SEQ ID NO: 86 - protein, 661 aa, synthetic construct
MAPKKKRKVGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 87 - protein, 661 aa, synthetic construct; identical to SEQ ID NO: 86

SEQ ID NO: 88 - protein, 7 aa, synthetic construct
PKKKRKV

SEQ ID NO: 89 - protein, 326 aa, synthetic construct
LTPDQVVAIASNIGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASHDGGRPALE

SEQ ID NO: 90 - protein, 326 aa, synthetic construct
LTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALE

SEQ ID NO: 91 - protein, 91 aa, synthetic construct
GSSLDDEHILSALLQSDDELVGEDSDSEVSDHVSEDDVQSDTEEAFIDEVHEVQPTSSGSEILDEQNVIEQPGSSLASNRILTLPQRTIRG

SEQ ID NO: 92 - protein, 755 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 93 - protein, 755 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 94 - DNA, 40 bp, synthetic construct
  source 1..40: mol_type = other DNA
acgcccacgcttacatctttaaagatgtaagcgtgggcgt

SEQ ID NO: 95 - DNA, 38 bp, synthetic construct
  source 1..38: mol_type = other DNA
acgcccacgctacatctttaaagatgtagcgtgggcgt

SEQ ID NO: 96 - DNA, 36 bp, synthetic construct
  source 1..36: mol_type = other DNA
acgcccacgctcatctttaaagatgagcgtgggcgt

SEQ ID NO: 97 - DNA, 34 bp, synthetic construct
  source 1..34: mol_type = other DNA
acgcccacgctctctttaaagagagcgtgggcgt

SEQ ID NO: 98 - DNA, 37 bp, synthetic construct
  source 1..37: mol_type = other DNA
ccctagaaagataatcattgcgtaaaattgacgcatg

SEQ ID NO: 99 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGLLVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 100 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVLTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 101 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLWDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 102 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMIYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 103 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRFDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 104 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSTRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 105 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDKFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 106 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHNCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 107 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGEHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 108 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPAKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 109 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMLCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 110 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDAGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 111 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGKGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 112 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTGTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 113 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKRLSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 114 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPIHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 115 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVAKNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 116 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEELKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 117 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGSSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 118 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPNKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 119 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSTCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 120 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSMDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 121 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCHEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 122 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASIDESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 123 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPEMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 124 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMIMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 125 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMLNIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 126 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRRKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 127 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSPFMRKRLEAPTLKRYLRDNISNILPKEVPGTSDDSTEEPVMKKRTYCTYCPSKIRRKANASCKKCKKVICREHNIDMCQSCF

SEQ ID NO: 128 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLPRYLRDNISNILPKEVPGTSDDSTEEPVMKKRTYCTYCPSKIRRKANASCKKCKKVICREHNIDMCQSCF

SEQ ID NO: 129 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIDRKANASCKKCKKVICREHNIDMCQSCF

SEQ ID NO: 130 - DNA, 11 bp, synthetic construct
  source 1..11: mol_type = other DNA
ttgtctctttt

SEQ ID NO: 131 - DNA, 6165 bp, synthetic construct
  source 1..6165: mol_type = other DNA
cgaaccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgtcatcgagctcgagatctggcgaaggcgatgggggtcttgaaggcgtgctggtactccacgatgcccagctcggtgttgctgtgcagctcctccacgcggcggaaggcgaacatggggcccccgttctgcaggatgctggggtggatggcgctcttgaagtgcatgtggctgtccaccacgaagctgtagtagccgccgtcgcgcaggctgaaggtgcgggcgaagctgcccaccagcacgttatcgcccatggggtgcaggtgctccacggtggcgttgctgcggatgatcttgtcggtgaagatcacgctgtcctcggggaagccggtgcccaccaccttgaagtcgccgatcacgcggccggcctcgtagcggtagctgaagctcacgtgcagcacgccgccgtcctcgtacttctcgatgcgggtgttggtgtagccgccgttgttaaccctagaaagatagtctgcgtaaaattgacgcatgcaatctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctacatcgcgaattgcagaaacatgcgtcaattttacgcatgattatctttaacgtacgtcacaatatgattatctttctagggttaatggcgtgcaggaaggggttctcgtagccgctggggtaggtgccgaagtggtagaagccgtagcccatcacgtggctcagcaggtaggggctgaaggtcagggcgcctttggtgctcttcatcttgttggtcatgcggccctgctcgggggtgccctctccgccgcccaccagctcgaactccacgccgttcagggtgccggtgatgcggcactcgatcttcatggcgggcatggtggcctgcaggactaaacctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggacagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaatttcaaatggctagagacttatcgaaagcagcgagacaggcgcgaaggtgccaccagattcgcacgcggcggccccagcgcccaggccaggcctcaactcaagcacgaggcgaaggggctcctaaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccaagcttggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtaccttctttgtctcttttgataaaacagactttaaaccaacaaagatcaaaagagacaagactgccaccatggtttctaagggcgaagccgtgatcaaagaattcatgcggttcaaggtgcacatggaaggcagcatgaacggccacgagttcgagatcgaaggcgaaggcgagggcagaccttatgagggaacacagaccgccaagctgaaagtgaccaaaggcggccctctgcctttcagctgggacattctgagccctcagtttatgtacggcagccgggccttcacaaagcaccctgccgatattcccgactactacaagcagagcttccccgagggcttcaagtgggagagagtgatgaacttcgaggacggcggagccgtgaccgtgacacaggatacaagcctggaagatggcaccctgatctacaaagtgaagctgcggggcaccaactttccacctgatggccccgtgatgcagaaaaagaccatgggctgggaagccagcaccgagagactgtatcctgaggatggcgtgctgaagggcgacatcaagatggccctgagactgaaggatggcggcagatacctggccgacttcaagaccacctacaaggccaagaaacccgtgcagatgcctggcgcctacaacgtggacagaaagctggacatcaccagccacaacgaggactacaccgtggtggaacagtacgagcggagcgaaggcagacactctacaggcggaatggacgagctgtacaagagccatggcttcccgccggaggtggaggagcaggatgatggcacgctgcccatgtcttgtgcccaggagagcgggatggaccgtcaccctgcagcctgtgcttctgctaggatcaatgtgtgattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaagctagcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggtt

SEQ ID NO: 132 - protein, 594 aa, synthetic construct
GGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMLCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 133 - protein, 594 aa, synthetic construct
GGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVHSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 134 - protein, 594 aa, synthetic construct
GGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 135 - protein, 594 aa, synthetic construct
GGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVHSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 136 - protein, 594 aa, synthetic construct
GGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMLCDSGTKYMINGMPYLGRGTQTNGVPLGEYYVKELSKPVHGSCRNITCDNWFTSIPLAKNLLQEPYKLTIVGTVHSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 137 - protein, 646 aa, synthetic construct
GGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMLCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 138 - protein, 646 aa, synthetic construct
GGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVHSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 139 - protein, 646 aa, synthetic construct
GGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCFAGGGSTEEPVMKKRTYCTYCPSKIRRKANASCKKCKKVICREHNIDMCQSCF

SEQ ID NO: 140 - protein, 646 aa, synthetic construct
GGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVHSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCFAGGGSTEEPVMKKRTYCTYCPSKIRRKANASCKKCKKVICREHNIDMCQSCF

SEQ ID NO: 141 - protein, 646 aa, synthetic construct
GGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMLCDSGTKYMINGMPYLGRGTQTNGVPLGEYYVKELSKPVHGSCRNITCDNWFTSIPLAKNLLQEPYKLTIVGTVHSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCFAGGGSTEEPVMKKRTYCTYCPSKIRRKANASCKKCKKVICREHNIDMCQSCF

SEQ ID NO: 142 - protein, 1141 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVHSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 143 - protein, 1141 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVHSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 144 - protein, 1141 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCFAGGGSTEEPVMKKRTYCTYCPSKIRRKANASCKKCKKVICREHNIDMCQSCF

SEQ ID NO: 145 - protein, 1141 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCFAGGGSTEEPVMKKRTYCTYCPSKIRRKANASCKKCKKVICREHNIDMCQSCF

SEQ ID NO: 146 - protein, 1141 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVHSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCFAGGGSTEEPVMKKRTYCTYCPSKIRRKANASCKKCKKVICREHNIDMCQSCF

SEQ ID NO: 147 - protein, 1141 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVHSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCFAGGGSTEEPVMKKRTYCTYCPSKIRRKANASCKKCKKVICREHNIDMCQSCF

SEQ ID NO: 148 - protein, 1141 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMLCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 149 - protein, 1141 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMLCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFAGGGSTEEPVMKKRTYCTYCPSKIRRKANASCKKCKKVICREHNIDMCQSCF

SEQ ID NO: 150 - protein, 1141 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMLCDSGTKYMINGMPYLGRGTQTNGVPLGEYYVKELSKPVHGSCRNITCDNWFTSIPLAKNLLQEPYKLTIVGTVHSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCFAGGGSTEEPVMKKRTYCTYCPSKIRRKANASCKKCKKVICREHNIDMCQSCF

SEQ ID NO: 151 - protein, 1141 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMLCDSGTKYMINGMPYLGRGTQTNGVPLGEYYVKELSKPVHGSCRNITCDNWFTSIPLAKNLLQEPYKLTIVGTVHSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCFAGGGSTEEPVMKKRTYCTYCPSKIRRKANASCKKCKKVICREHNIDMCQSCF

SEQ ID NO: 152 - DNA, 4005 bp, synthetic construct
  source 1..4005: mol_type = other DNA
ctagcggtctctttaaccctagaaagatagtctgcgtaaaattgacgcatgcattcttgaaatattgctctctctttctaaatgacgcgaatccgtcgctgtgcatttaggacatctcagtcgcgatatcggcctccaaggcctgcaggagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtttagtcctgcaggtcggccgccacgaccggtgccaccatgactgaatacaaaccaactgttcgcctggcaactcgtgatgatgttccacgtgcagttcgcaccctggctgctgcatttgctgactaccctgcaacccgtcacactgtggacccagaccgccacattgaacgtgtgactgaactgcaggagctgttcctgacccgtgtgggcctggacattggcaaagtgtgggtggcagatgatggtgctgctgtggcagtgtggaccacccctgaatctgttgaagctggtgcagtgtttgctgagattggcccacgcatggcagaactgtctggcagccgcctggcagcacaacagcagatggaaggtctgctggcaccacaccgcccaaaagaacctgcttggttcctggcaactgtgggtgtgagccctgaccaccagggtaagggcctgggctctgcagtggtgctgcctggtgtggaagcagctgaacgtgcaggtgtgcctgctttcctggagacctcagctccacgcaacctgcctttctatgaacgcctgggcttcactgtgactgctgatgtggaagtgccagaaggcccacgcacttggtgcatgactcgcaaaccaggtgctagccttggctccggcgagggcaggggaagtcttctaacatgcggggacgtggaggaaaatcccggcccactcggctacccctacgacgtgcccgactacgccagcggcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttcgaactagatgctgtcgaccgatgcccttgagagccttcaacccagtcagctccttccggtgggcgcggggcaatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgactatcgtcgccgcacttatcatgcgtcaattttacgcaatgactatctttctagggttaaagagacctgtacatacctaatgatttttatcaaaatcattaagttaaggtagatacacatcttgtcatatgatcaaatggtttcgccaaaaatcaataatcagacaacaagatgtgcgaactcgatattttacacgactctctttaccaattctaccacaacgtggatcagcagttcaacctgttgatagtacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccaaaaaaaagcccgctcattaggcgggctgcgcccttaagg

SEQ ID NO: 153 - DNA, 78 bp, synthetic construct
  source 1..78: mol_type = other DNA
caggcaggatgaatctgtgctctgatccctgaggcatttaatatgttcttattattagaagctcagatgcaaagagct

SEQ ID NO: 154 - DNA, 10 bp, synthetic construct
  source 1..10: mol_type = other DNA
tctgtgctct

SEQ ID NO: 155 - DNA, 11 bp, synthetic construct
  source 1..11: mol_type = other DNA
tctgtgctctg

SEQ ID NO: 156 - DNA, 14 bp, synthetic construct
  source 1..14: mol_type = other DNA
tgaatctgtgctct

SEQ ID NO: 157 - DNA, 15 bp, synthetic construct
  source 1..15: mol_type = other DNA
tgaatctgtgctctg

SEQ ID NO: 158 - DNA, 10 bp, synthetic construct
  source 1..10: mol_type = other DNA
tgagcttcta

SEQ ID NO: 159 - DNA, 11 bp, synthetic construct
  source 1..11: mol_type = other DNA
tgagcttctaa

SEQ ID NO: 160 - DNA, 12 bp, synthetic construct
  source 1..12: mol_type = other DNA
tctgagcttcta

SEQ ID NO: 161 - DNA, 13 bp, synthetic construct
  source 1..13: mol_type = other DNA
tctgagcttctaa

SEQ ID NO: 162 - DNA, 16 bp, synthetic construct
  source 1..16: mol_type = other DNA
tgcatctgagcttcta

SEQ ID NO: 163 - DNA, 17 bp, synthetic construct
  source 1..17: mol_type = other DNA
tgcatctgagcttctaa

SEQ ID NO: 164 - DNA, 17 bp, synthetic construct
  source 1..17: mol_type = other DNA
ttgcatctgagcttcta

SEQ ID NO: 165 - DNA, 18 bp, synthetic construct
  source 1..18: mol_type = other DNA
ttgcatctgagcttctaa

SEQ ID NO: 166 - DNA, 18 bp, synthetic construct
  source 1..18: mol_type = other DNA
tttgcatctgagcttcta

SEQ ID NO: 167 - DNA, 19 bp, synthetic construct
  source 1..19: mol_type = other DNA
tttgcatctgagcttctaa

SEQ ID NO: 168 - DNA, 20 bp, synthetic construct
  source 1..20: mol_type = other DNA
tctttgcatctgagcttcta

SEQ ID NO: 169 - DNA, 21 bp, synthetic construct
  source 1..21: mol_type = other DNA
tctttgcatctgagcttctaa

SEQ ID NO: 170 - protein, 326 aa, synthetic construct
LTPDQVVAIASHDGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASHDGGRPALE

SEQ ID NO: 171 - protein, 326 aa, synthetic construct
LTPDQVVAIASNGGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIASNGGGRPALE

SEQ ID NO: 172 - protein, 1202 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLGGGGSVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMLCDSGTKYMINGMPYLGRGTQTNGVPLGEYYVKELSKPVHGSCRNITCDNWFTSIPLAKNLLQEPYKLTIVGTVHSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCFAGGGSTEEPVMKKRTYCTYCPSKIRRKANASCKKCKKVICREHNIDMCQSCF

SEQ ID NO: 173 - protein, 1202 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGFSLASNRILTLGGGGSVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMLCDSGTKYMINGMPYLGRGTQTNGVPLGEYYVKELSKPVHGSCRNITCDNWFTSIPLAKNLLQEPYKLTIVGTVHSNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCFAGGGSTEEPVMKKRTYCTYCPSKIRRKANASCKKCKKVICREHNIDMCQSCF

SEQ ID NO: 174 - DNA, 4293 bp, synthetic construct
  source 1..4293: mol_type = other DNA
ccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgtcgacgttggtaaagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcttaaccctagaaagatagtctgcgtaaaattgacgcatgcattcttgaaatattgctctctctttctaaatagcgcgaatccgtcgctgtgcatttaggacatctcagtcgcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaaacatgcgtcaattttacgcagactatctttctagggttaagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaatcgagtctagagggcccgagatctttttccctctgccaaaaattatggggacatcatgaagccccttgagcatctgacttctggctaataaaggaaatttattttcattgcaatagtgtgttggaattttttgtgtctctcactcggaaggacataagggcggccgctagc

SEQ ID NO: 175 - DNA, 52 bp, synthetic construct
  source 1..52: mol_type = other DNA
tcaagaaggactttcataagtcacttaaagatttacaggagagcactgctaa

(19) United States
(12) Patent Application Publication
(10) Pub. No.: US 2026/0055383 A1
(43) Pub. Date: Feb. 26, 2026

(54) TRANSPOSASES AND USES THEREOF

(71) Applicant: Poseida Therapeutics, Inc., San Diego, CA (US)

(72) Inventors: Joseph S. LUCAS, San Diego, CA (US); Olga BATALOV, San Diego, CA (US); Michelle Jiajia XU, San Diego, CA (US); Blair B. MADISON, San Diego, CA (US); Ming-Yue LEE, San Diego, CA (US)

(21) Appl. No.: 19/382,756

(22) Filed: Nov. 7, 2025

Related U.S. Application Data
(63) Continuation of application No. PCT/US2024/028629, filed on May 9, 2024.
(60) Provisional application No. 63/501,233, filed on May 10, 2023.
(60) Provisional application No. 63/604,996, filed on Dec. 1, 2023.

(51) Int. Cl. C12N 9/16; A61K 48/00

(52) U.S. Cl. CPC C12N 9/16; A61K 48/0066; C07K 2319/01; C07K 2319/09; C07K 2319/81

(57) ABSTRACT

This disclosure generally relates to transposase domains, in particular, transposase domains comprising amino terminal deletions, as well as transposase domains forming obligate heterodimers and transposase domains comprising DNA targeting domains.

### CROSS-REFERENCE TO RELATED APPLICATIONS

This application is a continuation of International Patent Application No. PCT/US2024/028629, filed May 9, 2024, which claims the benefit of U.S. Provisional Application No. 63/604,996, filed Dec. 1, 2023, and U.S. Provisional Application No. 63/501,233, filed May 10, 2023. Each of the foregoing applications is incorporated herein by reference in its entirety.

### REFERENCE TO SEQUENCE LISTING SUBMITTED ELECTRONICALLY

The instant application contains a Sequence Listing which has been submitted in XML format via Patent Center and is herein incorporated by reference in its entirety. Said XML copy, created on Nov. 7, 2025 is named “000218-0144-101-SL.xml” and is 272,411 bytes in size.

### FIELD

This disclosure generally relates to transposase domains, in particular, transposase domains comprising dual cysteine rich domains (CRD), as well as transposase domains comprising N-terminal deletions, transposase domains forming obligate heterodimers and fusion proteins comprising the transposes domains and DNA targeting domains. Also provided are methods of use of the fusion proteins for site-specific transposition.

### BACKGROUND

Transposases may be used to introduce non-endogenous DNA sequences into genomic DNA, and are in many ways advantageous to other methods of gene editing.

PiggyBac transposase consists of several protein domains. Binding to the transposon's ITRs is mediated by the DNA dimerization and binding domain (DDBD) along with the cysteine rich C-terminal domain (CRD). The DDBD and CRD serve dual roles, as they are also involved in protein dimerization. Binding of the transposase dimer to the ITRs positions the catalytic domain of the transposase on the TTAA cleavage sites which flank the transposon. A second transposase dimer is thought to bind further along the ITRs, distal to TTAA. While this dimer is not involved in catalyzing the transposase reaction, it's presence may stabilize the hairpin structure of the transposon in the transposasome.

The PiggyBac transposase was originally isolated from the genome of the cabbage looper moth Trichoplusia ni. Since then, several point mutations have been identified that boost the transposition activity of PiggyBac when used as a genome editing tool. For example, the Super PiggyBac (SPB) transposase comprises four point mutations, I30V, G165S, M282V, and N538K. Another PiggyBac transposase version called “Hyperactive PiggyBac transposase” or hyPBase was described (Yusa et al. (2011), PNAS 108(4):1531-1536). HyPBase contains the four point mutations found in SPB (I30V, G165S, M282V, N538K) plus three additional mutations (S103P, S509G, N571S).

Furthermore, an integration deficient (or excision only) variant of PiggyBac, called “PBx,” has been described that contains two mutations (R372A, K375A). Converting the positively charged amino acids to uncharged residues at this position results in a transposase that cannot interact with DNA targets. PBx can be built on top of the SPB or HyPBase hyperactive mutations and is incorporated into ssSPB. Additionally, in the context of PBx (but not SPB), the D450N mutation adds a further boost to excision activity. Alternative versions of PBx can be created by converting positions 372 and 375 to alternative amino acids, in theory allowing one to titrate the strength of interaction with target DNA. By using site-saturation mutagenesis, the R372H mutations was found to improve integration efficiency of ssSPB.

However, there remains an unmet need for site-specific transposases for use in e.g., gene editing. Provided herein are improved versions of piggyBac transposases that comprise dual CRD domains, point mutations with the potential to improve protein thermostability, as well as additional hyperactive mutations.

### SUMMARY

In one aspect, provided herein is a fusion protein comprising, in N-terminal to C-terminal order: a nuclear localization signal (NLS), a DNA targeting domain, and a first transposase domain comprising an amino acid sequence of any of SEQ ID NOs: 28-69. In some embodiments, the first transposase domain comprises an amino acid sequence of any of SEQ ID NOs: 28-48. In some embodiments, the first transposase domain comprises an amino acid sequence of any of SEQ ID NOs: 49-69. In some embodiments, the first transposase domain comprises an amino acid sequence of SEQ ID NO: 30 or 38. In some embodiments, the first transposase domain comprises an amino acid sequence of SEQ ID NO: 51 or 59.

In some embodiments, the DNA targeting domain comprises one or more Zinc Finger Motifs. In some embodiments, the DNA targeting domain comprises the sequence of SEQ ID NO: 74. In some embodiments, the DNA targeting domain comprises one or more TAL domains. In some embodiments, the DNA targeting domain binds to a nucleic acid sequence encoding GFP, a LINE1 repeat element, or the zinc finger 268 (ZFM268) binding site.

In another aspect, provided herein is a fusion protein, comprising: (a) a TAL Array; and (b) a modified Super piggyBac transposase (“SPB”) comprising a N-terminal deletion and a second cysteine rich domain (CRD) fused to the C-terminus of the SPB transposase; wherein the C-terminus of the TAL Array is fused to the N-terminal amino acid of the N-terminal deleted SPB to generate a TAL Array-N-terminal deleted SPB fusion protein. In some embodiments, the fusion protein further comprises an GS or GGGS linker positioned between the TAL Array and the N-terminal deleted SPB. In some embodiments, the SPB comprises a N-terminal deletion comprising a deletion of amino acids 1-83, 1-84, 1-85, 186, 1-87, 1-88, 1-89, 1-90, 1-91, 1-92, 1-93, 1-94, 1-95, 1-96, 1-97, 1-98, 1-99, 1-100, 1-101, 1-102 or 1-103.

In another aspect, provided herein is a fusion protein, comprising: (a) a TAL Array; and (b) a modified Super piggyBac transposase (“SPB”) comprising a N-terminal deletion, one or more integration-deficient PBx mutations and a second cysteine rich domain (CRD) fused to the C-terminus of the SPB transposase; wherein the C-terminus of the TAL Array is fused to the N-terminal amino acid of the N-terminal deleted SPB to generate a TAL Array-N-terminal deleted SPB fusion protein. In some embodiments, the fusion protein further comprise a GS or a GGGS linker positioned between the TAL Array and the N-terminal deleted SPB. In some embodiments, the modified Super piggyBac transposase comprises the amino acid sequence of any of SEQ ID Nos: 28-69.

In another aspect, provided herein is a fusion protein comprising, in N-terminal to C-terminal order: a DNA targeting domain and a first transposase domain comprising the sequence set forth in SEQ ID NO: 1 or 3, wherein the first transposase domain comprises a deletion of the 83-103 most N-terminal amino acids of SEQ ID NO: 1 or 73. In some embodiments, the DNA targeting domain comprises one or more Zinc Finger Motifs. In some embodiments, the DNA targeting domain comprises one or more TAL domains. In some embodiments, the DNA targeting domain binds to a nucleic acid sequence encoding GFP, zinc finger 268 (ZFM268), phenylalanine hydroxylase (PAH), beta-2-microglobulin (B2M) or a LINE1 repeat element.

In some embodiments, the first transposase domain and the DNA targeting domain are connected by a linker. In some embodiments, the linker comprises the sequence GGGGS.

In some embodiments, the first transposase domain comprises an N-terminal deletion of amino acids 1-83, 1-84, 1-85, 186, 1-87, 1-88, 1-89, 1-90, 1-91, 1-92, 1-93, 1-94, 1-95, 1-96, 1-97, 1-98, 1-99, 1-100, 1-101, 1-102 or 1-103. In some embodiments, the first transposase domain comprises (a) at least one mutation selected from the group consisting of M185R, M185K, D197K, D197R, D198K, D198R, D201K, and D201R; or (b) at least one mutation selected from the group consisting of L204D, L204E, K500D, K500E, R504E, and R504D relative to SEQ ID NO: 1 or 73, with numbering beginning at the 12^{th} residue of SEQ ID NO: 1 and the first residue of SEQ ID NO: 73.

In some embodiments, the fusion protein of further comprises a second transposase domain C-terminal to the first transposase domain, wherein the second transposase domain comprises the sequence set forth in SEQ ID NO: 1 or 73. In some embodiments, the second transposase domain comprises a deletion of N-terminal amino acids 1-83, 1-84, 1-85, 186, 1-87, 1-88, 1-89, 1-90, 1-91, 1-92, 1-93, 1-94, 1-95, 1-96, 1-97, 1-98, 1-99, 1-100, 1-101, 1-102 or 1-103 of SEQ ID NO: 1 or 73. In some embodiments, the second transposase domain comprises (a) at least one mutation selected from the group consisting of M185R, M185K, D197K, D197R, D198K, D198R, D201K, and D201R; or (b) at least one mutation selected from the group consisting of L204D, L204E, K500D, K500E, R504E, and R504D relative to SEQ ID NO: 1 or 73 with numbering beginning at the 12^{th} residue of SEQ ID NO: 1 and the first residue of SEQ ID NO: 73.

In another aspect, provided herein is a polynucleotide comprising a nucleic acid sequence encoding a fusion protein described herein.

In another aspect, provided herein is a transposon, comprising symmetrical left end (LE) and right end (RE) inverted terminal repeat sequences (ITRs), wherein the nucleotide sequences of the LE ITR and the RE ITR are each SEQ ID NO: 6.

In another aspect, provided herein is a transposon, comprising symmetrical left end (LE) and right end (RE) inverted terminal repeat sequences (ITRs), wherein the nucleotide sequence of the LE ITR comprises SEQ ID NO: 6 and the nucleotide sequence of the RE ITR comprises SEQ ID NO: 7, SEQ ID NO: 8, SEQ ID NO: 9, SEQ ID NO: 10, SEQ ID NO: 21, or SEQ ID NO: 98. In some embodiments, the transposon comprises a nucleotide sequence encoding a therapeutic protein. In some embodiments, the transposon comprises a promoter sequence controlling expression of the therapeutic protein.

In another aspect, provided herein is a method for site specific integration of a therapeutic gene into one or more genomic locus of a cell, comprising co-introducing into the cell a transposon described herein and a polynucleotide described herein.

In another aspect, provided herein is a method of modifying the genome of a cell, the method comprising: providing the cell with a fusion protein described herein, wherein the cell comprises a modified binding site comprising, in 5′ to 3′ order, the reverse of the sequence of a target site for the DNA targeting domain, a first spacer, a TTAA target integration site for SPB, a second spacer, and the complement of the sequence of the target site for the DNA targeting domain.

### BRIEF DESCRIPTION OF DRAWINGS

FIG. 1 shows a schematic depiction of the dual reporter plasmid design used to confirm the rates of excision and integration using each mutant transposon. Using an H-2kk GFP transposon reporter (Reporter 1), an increase in H2kk expression is observed if there is an increase in excision of the transposon. Using Reporter 2, an increase in GFP expression is observed if there is an increase in the integration of the transposon. In an alternative design of Reporter 2, an increase in Firefly luciferase expression is observed if there is an increase in excision of the transposon and an increase in NanoLuc is observed if there is an increase in the integration of the transposon.

FIG. 2 is a schematic depiction of the dual reporter plasmid design used to confirm the rates of excision and integration of transposases comprising dual CRD domains using transposons comprising a wild type RE ITR or a modified symmetrical RE ITRs (0 bp) or a modified symmetrical RE ITR comprising an additional, 1 bp, 2 bp, 3 bp or 3 bp separating the DDBD and CRD binding sites or a modified symmetrical RE ITR comprising an additional 3 bp separating the DDBD and CRD binding sites and using the DDBD binding domain binding site of the RE ITR (1 bp difference).

FIG. 3 shows excision and integration activity of TAL-ssSPB mutations comprising thermostability mutations.

FIG. 4 illustrates TAL target sequences, TAL target length, and spacing from the TTAA integration site for the constructs described in Example 8.

### DETAILED DESCRIPTION

Provided herein are transposase domains and fusion proteins comprising the same, in particular, transposase domains comprising N-terminal deletions and dual C-terminal cysteine rich domains (CRDs). The fusion proteins comprising said transposase domains may be further mutated so that they form obligate heterodimers. Also provided are methods of making the transposase domains and fusion proteins, cells that are modified using the fusion proteins provided herein and methods of treatment using such cells.

Transposase domains provided herein may be, for example, wildtype transposase domains or integration deficient (excision only) transposase domains.

Also provided herein are fusion proteins comprising one or more transposase domains and a DNA targeting domain. In some embodiment, the fusion protein further comprises a protein stabilization domain.

Also provided herein are transposons comprising symmetrical left end (LE) and right end (RE) inverted terminal repeat sequence (ITRs).

### Transposase Domains Comprising Dual Cysteine Rich Domains (CRD)

The dimerization and DNA binding domain (DDBD) allows the transposase to bind to the inverted terminal repeats (ITRs) at the ends of the transposon and is also involved in protein dimerization. The catalytic domain catalyzes the transposition reaction while the insertion domain allows the transposase to interact with the target DNA in which the transposon integrates. At the C-terminus, a cysteine rich domain (CRD) is attached to the rest of the protein by a linker approximately 20 amino acids in length. Like the DDBD, the CRD is also involved in ITR binding and protein dimerization. Upon binding of PiggyBac transpose to the left end (LE) and right end (RE) ITRs, protein dimerization brings the two ITRs together to form a synaptic hairpin complex. This arrangement is required for transposition to occur as the transposase cuts the flanking TTAA sequences in trans.

The DDBD and the CRD bind to the ITRs in a sequence-specific manner. The DDBD interacts with about 10 bp of DNA located 6 bp in from the TTAA sequences flanking the transposon. Binding of the DDBD is symmetrical, with the DDBD of one transposase monomer binding the LE ITR and the DDBD of the second monomer binding the RE ITR. The CRD domains of the first dimer bind with a 19 bp sequence of the LE ITR found immediately distal to the DDBD binding site. CRD binding is asymmetric with both CRD domains of the first dimer interacting with the 19 bp sequence on the LE ITR only. The RE ITR contains a second DDBD binding sequence followed by a 19 bp CRD binding sequence starting 34 bp in from the TTAA. The first dimer that binds proximal to the TTAAs catalyzes the transposition reaction.

In one aspect, provided herein are transposase domains comprising a second cysteine rich domain (CRD). In some embodiments, the second CRD domain is linked to the C-terminus to generate transposase domains comprising dual CRDs. In some embodiments, the transposase domains comprising dual CRDs comprise an N-terminal deletion. In some embodiments, the transposase domain comprising dual CRDs is a piggyBac transposase domain. In some embodiments, the piggyBac transposase domain is a hyperactive piggyBac transposase domain. In preferred embodiments, the transposase domain comprising dual CRDs is a Super piggyBac® transposase domains (SPB). Non-limiting examples of SPB transposases are described in detail in U.S. Pat. Nos. 6,218,182; 6,962,810; 8,399,643 and PCT Publication No. WO 2010/099296, each of which is incorporated herein by reference in its entirety for examples of transposase domains that may be used in connection with the fusion proteins described herein.

In some embodiments, the transposase domain comprising dual CRDs is a Super PiggyBac transposase (SPB) domain. An exemplary wildtype SPB sequence with an NLS is shown in SEQ ID NO: 1 with the NLS shown in italics, hyperactive mutations shown in bold, and the Cysteine Rich Domain (CRD) underlined. The numbering of sequence of the SPB transposase domain for the purpose of describing deletions and mutations begins at residue 12 of SEQ ID NO: 1:

(SEQ ID NO: 1)
MAPKKKRKVGGGGSSLDDEHILSALLQSDDELVGEDSDSEVSDHVSEDDVQSDTEEAFI
DEVHEVQPTSSGSEILDEQNVIEQPGSSLASNRILTLPQRTIRGKNKHCWSTSKSTRRSRV
SALNIVRSQRGPTRMCRNIYDPLLCFKLFFTDEIISEIVKWTNAEISLKRRESMTSATFRDT
NEDEIYAFFGILVMTAVRKDNHMSTDDLFDRSLSMVYVSVMSRDRFDFLIRCLRMDDK
SIRPTLRENDVFTPVRKIWDLFIHQCIQNYTPGAHLTIDEQLLGFRGRCPFRVYIPNKPSK
YGIKILMMCDSGTKYMINGMPYLGRGTQTNGVPLGEYYVKELSKPVHGSCRNITCDNW
FTSIPLAKNLLQEPYKLTIVGTVRSNKREIPEVLKNSRSRPVGTSMFCFDGPLTLVSYKPK
PAKMVYLLSSCDEDASINESTGKPQMVMYYNQTKGGVDTLDQMCSVMTCSRKTNRWP
MALLYGMINIACINSFIIYSHNVSSKGEKVQSRKKFMRNLYMSLTSSFMRKRLEAPTLKR
YLRDNISNILPKEVPGTSDDSTEEPVMKKRTYCTYCPSKIRRKANASCKKCKKVICREHN
IDMCQSCF

An exemplary wildtype SPB sequence comprising a second, C-terminal CRD domain attached via an AGGG peptide linker sequence (SEQ ID NO: 27) is shown in SEQ ID NO: 3 with the NLS shown in italics, hyperactive mutations shown in bold, the linker sequence shown in lower case font and each of the dual Cysteine Rich Domains (CRD) underlined. The numbering of sequence of the SPB transposase domain for the purpose of describing deletions and mutations begins at residue 12 of SEQ ID NO: 3:

(SEQ ID NO: 3)
MAPKKKRKVGGGGSSLDDEHILSALLQSDDELVGEDSDSEVSDHVSEDDVQSDTEEAFI
DEVHEVQPTSSGSEILDEQNVIEQPGSSLASNRILTLPQRTIRGKNKHCWSTSKSTRRSRV
SALNIVRSQRGPTRMCRNIYDPLLCFKLFFTDEIISEIVKWTNAEISLKRRESMTSATFRDT
NEDEIYAFFGILVMTAVRKDNHMSTDDLFDRSLSMVYVSVMSRDRFDFLIRCLRMDDK
SIRPTLRENDVFTPVRKIWDLFIHQCIQNYTPGAHLTIDEQLLGFRGRCPFRVYIPNKPSK
YGIKILMMCDSGTKYMINGMPYLGRGTQTNGVPLGEYYVKELSKPVHGSCRNITCDNW
FTSIPLAKNLLQEPYKLTIVGTVRSNKREIPEVLKNSRSRPVGTSMFCFDGPLTLVSYKPK
PAKMVYLLSSCDEDASINESTGKPQMVMYYNQTKGGVDTLDQMCSVMTCSRKTNRWP
MALLYGMINIACINSFIIYSHNVSSKGEKVQSRKKFMRNLYMSLTSSFMRKRLEAPTLKR
YLRDNISNILPKEVPGTSDDSTEEPVMKKRTYCTYCPSKIRRKANASCKKCKKVICREHN
IDMCQSCFagggSTEEPVMKKRTYCTYCPSKIRRKANASCKKCKKVICREHNIDMCQSCF

The amino acid sequence of an SPB transposase domain comprising dual CRD domains not comprising an NLS is set forth in SEQ ID NO: 73:

(SEQ ID NO: 73)
GGSSLDDEHILSALLQSDDELVGEDSDSEVSDHVSEDDVQSDTEEAFIDEVHEVQPTSSG
SEILDEQNVIEQPGSSLASNRILTLPQRTIRGKNKHCWSTSKSTRRSRVSALNIVRSQRGPT
RMCRNIYDPLLCFKLFFTDEIISEIVKWTNAEISLKRRESMTSATFRDTNEDEIYAFFGILV
MTAVRKDNHMSTDDLFDRSLSMVYVSVMSRDRFDFLIRCLRMDDKSIRPTLRENDVFT
PVRKIWDLFIHQCIQNYTPGAHLTIDEQLLGFRGRCPFRVYIPNKPSKYGIKILMMCDSGT
KYMINGMPYLGRGTQTNGVPLGEYYVKELSKPVHGSCRNITCDNWFTSIPLAKNLLQEP
YKLTIVGTVRSNKREIPEVLKNSRSRPVGTSMFCFDGPLTLVSYKPKPAKMVYLLSSCDE
DASINESTGKPQMVMYYNQTKGGVDTLDQMCSVMTCSRKTNRWPMALLYGMINIACI
NSFIIYSHNVSSKGEKVQSRKKFMRNLYMSLTSSFMRKRLEAPTLKRYLRDNISNILPKE
VPGTSDDSTEEPVMKKRTYCTYCPSKIRRKANASCKKCKKVICREHNIDMCQSCFAGGG
STEEPVMKKRTYCTYCPSKIRRKANASCKKCKKVICREHNIDMCQSCF.

The transposase domains used in the fusion proteins described herein can be isolated or derived from an insect, vertebrate, crustacean or urochordate as described in more detail in PCT Publication No. WO 2019/173636 and PCT/US2019/049816. In preferred aspects, the SPB transposase domain is isolated or derived from the insect Trichoplusia ni (GenBank Accession No. AAA87375) or Bombyx mori (GenBank Accession No. BAD 11135).

In some embodiments, the transposase domain is integration deficient. An integration deficient transposase domain is a transposase that can excise its corresponding transposon, but that integrates the excised transposon at a lower frequency than a corresponding wild type transposase. Examples of integration deficient transposases are disclosed in U.S. Pat. Nos. 6,218,185; 6,962,810, 8,399,643 and International Patent Application Publication No. WO 2019/173636 each of which is incorporated herein by reference in its entirety for examples of transposase domains that may be used in connection with the fusion proteins described herein. A list of integration deficient amino acid substitutions is disclosed in U.S. Pat. No. 10,041,077, which is incorporated herein by reference in its entirety for examples of mutations that may be introduced into the transposase domains described herein. A wildtype SPB may be rendered integration deficient by introducing mutations, for example, K93A, R372A, K375A, R376A and/or D450N (relative to SEQ ID NO: 1, with numbering beginning at residue 12). It is believed that the introduction of mutations R372A, K375A, R376A and D450N renders the transposase integration deficient, but retains the excision function. The amino acid sequence of an integration deficient PBx transposase domain not comprising an NLS is set forth in SEQ ID NO: 70:

(SEQ ID NO: 70)
GGSSLDDEHILSALLQSDDELVGEDSDSEVSDHVSEDDVQSDTEEAFIDEVHEVQPTSSG
SEILDEQNVIEQPGSSLASNRILTLPQRTIRGKNKHCWSTSKSTRRSRVSALNIVRSQRGPT
RMCRNIYDPLLCFKLFFTDEIISEIVKWTNAEISLKRRESMTSATFRDTNEDEIYAFFGILV
MTAVRKDNHMSTDDLFDRSLSMVYVSVMSRDRFDFLIRCLRMDDKSIRPTLRENDVFT
PVRKIWDLFIHQCIQNYTPGAHLTIDEQLLGFRGRCPFRVYIPNKPSKYGIKILMMCDSGT
KYMINGMPYLGRGTQTNGVPLGEYYVKELSKPVHGSCRNITCDNWFTSIPLAKNLLQEP
YKLTIVGTVASNAREIPEVLKNSRSRPVGTSMFCFDGPLTLVSYKPKPAKMVYLLSSCDE
DASINESTGKPQMVMYYNQTKGGVDTLNQMCSVMTCSRKTNRWPMALLYGMINIACI
NSFIIYSHNVSSKGEKVQSRKKFMRNLYMSLTSSFMRKRLEAPTLKRYLRDNISNILPKE
VPGTSDDSTEEPVMKKRTYCTYCPSKIRRKANASCKKCKKVICREHNIDMCQSCF.

The amino acid sequence of an integration deficient PBx transpose domain not comprising an NL S but comprising a second CRD domain sequence (SEQ ID NO: 2) linked to the C-terminus of PBx sequence via an AGGG linker sequence (SEQ ID NO: 27) is set forth in SEQ ID NO: 71:

(SEQ ID NO: 71)
GGSSLDDEHILSALLQSDDELVGEDSDSEVSDHVSEDDVQSDTEEAFIDEVHEVQPTSSG
SEILDEQNVIEQPGSSLASNRILTLPQRTIRGKNKHCWSTSKSTRRSRVSALNIVRSQRGPT
RMCRNIYDPLLCFKLFFTDEIISEIVKWTNAEISLKRRESMTSATFRDTNEDEIYAFFGILV
MTAVRKDNHMSTDDLFDRSLSMVYVSVMSRDRFDFLIRCLRMDDKSIRPTLRENDVFT
PVRKIWDLFIHQCIQNYTPGAHLTIDEQLLGFRGRCPFRVYIPNKPSKYGIKILMMCDSGT
KYMINGMPYLGRGTQTNGVPLGEYYVKELSKPVHGSCRNITCDNWFTSIPLAKNLLQEP
YKLTIVGTVASNAREIPEVLKNSRSRPVGTSMFCFDGPLTLVSYKPKPAKMVYLLSSCDE
DASINESTGKPQMVMYYNQTKGGVDTLNQMCSVMTCSRKTNRWPMALLYGMINIACI
NSFIIYSHNVSSKGEKVQSRKKFMRNLYMSLTSSFMRKRLEAPTLKRYLRDNISNILPKE
VPGTSDDSTEEPVMKKRTYCTYCPSKIRRKANASCKKCKKVICREHNIDMCQSCFAGGG
STEEPVMKKRTYCTYCPSKIRRKANASCKKCKKVICREHNIDMCQSCF

The amino acid sequence of an integration deficient PBx transpose domain not comprising an NL S but comprising a second CRD domain sequence (SEQ ID NO: 14) linked to the C-terminus of PBx sequence is set forth in SEQ ID NO: 72:

(SEQ ID NO: 72)
GGSSLDDEHILSALLQSDDELVGEDSDSEVSDHVSEDDVQSDTEEAFIDEVHEV
QPTSSGSEILDEQNVIEQPGSSLASNRILTLPQRTIRGKNKHCWSTSKSTRRSRVSALNIVR
SQRGPTRMCRNIYDPLLCFKLFFTDEIISEIVKWTNAEISLKRRESMTSATFRDTNEDEIYA
FFGILVMTAVRKDNHMSTDDLFDRSLSMVYVSVMSRDRFDFLIRCLRMDDKSIRPTLRE
NDVFTPVRKIWDLFIHQCIQNYTPGAHLTIDEQLLGFRGRCPFRVYIPNKPSKYGIKILMM
CDSGTKYMINGMPYLGRGTQTNGVPLGEYYVKELSKPVHGSCRNITCDNWFTSIPLAK
NLLQEPYKLTIVGTVASNAREIPEVLKNSRSRPVGTSMFCFDGPLTLVSYKPKPAKMVYL
LSSCDEDASINESTGKPQMVMYYNQTKGGVDTLNQMCSVMTCSRKTNRWPMALLYG
MINIACINSFIIYSHNVSSKGEKVQSRKKFMRNLYMSLTSSFMRKRLEAPTLKRYLRDNIS
NILPKEVPGTSDDSTEEPVMKKRTYCTYCPSKIRRKANASCKKCKKVICREHNIDMCQS
CFGTSDDSTEEPVMKKRTYCTYCPSKIRRKANASCKKCKKVICREHNIDMCQSCF

### Transposase Domains Comprising N-terminal Deletions

In some embodiments, provided herein are dual CRD transposase domains (e.g., SPB transposase domains or PBx transposase domains) comprising a deletion of a portion of the amino terminus (also referred to as the “N-terminus” or the “N-terminal Domain,” or “NTD) of the transposase domain. Without wishing to be bound by theory, it is believed that, in the context of a tandem dimer transposase (or a dimer comprising two fusion proteins described herein) the N-terminal domain of a transposase (e.g., SPB) may introduce steric hindrance between the two dimers of a tandem dimer, or between two pairs of dimers, or between a dimer and the DNA.

In some embodiments, the deleted portion of the N-terminus is about 20 amino acids, about 40 amino acids, about 60 amino acids, about 80 amino acids, about 100 amino acids or about 115 amino acids. In some embodiments, the deleted portion of the N-terminus is about 15-25 amino acids, about 25-35 amino acids, about 35-45 amino acids, about 45-55 amino acids, about 55-65 amino acids, about 65-75 amino acids, about 75-85 amino acids, about 85-95 amino acids, about 95-105 amino acids, or about 105-120 amino acids.

In some embodiments, the transposase domain comprises a deletion of amino acids 1-83 of the N-terminus relative to SEQ ID NOs: 71-73. In some embodiments, the transposase domain comprises a deletion of amino acids 1-84 of the N-terminus relative to SEQ ID NOs: 71-73. In some embodiments, the transposase domain comprises a deletion of amino acids 1-85 of the N-terminus relative to SEQ ID NOs: 71-73. In some embodiments, the transposase domain comprises a deletion of amino acids 1-86 of the N-terminus relative to SEQ ID NOs: 71-73. In some embodiments, the transposase domain comprises a deletion of amino acids 1-87 of the N-terminus relative to SEQ ID NOs: 71-73. In some embodiments, the transposase domain comprises a deletion of amino acids 1-88 of the N-terminus relative to SEQ ID NOs: 71-73. In some embodiments, the transposase domain comprises a deletion of amino acids 1-89 of the N-terminus relative to SEQ ID NOs: 71-73. In some embodiments, the transposase domain comprises a deletion of amino acids 1-90 of the N-terminus relative to SEQ ID NOs: 71-73. In some embodiments, the transposase domain comprises a deletion of amino acids 1-91 of the N-terminus relative to SEQ ID NOs: 71-73. In some embodiments, the transposase domain comprises a deletion of amino acids 1-92 of the N-terminus relative to SEQ ID NOs: 71-73. In some embodiments, the transposase domain comprises a deletion of amino acids 1-93 of the N-terminus relative to SEQ ID NOs: 71-73. In some embodiments, the transposase domain comprises a deletion of amino acids 1-94 of the N-terminus relative to SEQ ID NOs: 71-73. In some embodiments, the transposase domain comprises a deletion of amino acids 1-95 of the N-terminus relative to SEQ ID NOs: 71-73. In some embodiments, the transposase domain comprises a deletion of amino acids 1-96 of the N-terminus relative to SEQ ID NOs: 71-73. In some embodiments, the transposase domain comprises a deletion of amino acids 1-97 of the N-terminus relative to SEQ ID NOs: 71-73. In some embodiments, the transposase domain comprises a deletion of amino acids 1-98 of the N-terminus relative to SEQ ID NOs: 71-73. In some embodiments, the transposase domain comprises a deletion of amino acids 1-99 of the N-terminus relative to SEQ ID NOs: 71-73. In some embodiments, the transposase domain comprises a deletion of amino acids 1-100 of the N-terminus relative to SEQ ID NOs: 71-73. In some embodiments, the transposase domain comprises a deletion of amino acids 1-101 of the N-terminus relative to SEQ ID NOs: 71-73. In some embodiments, the transposase domain comprises a deletion of amino acids 1-102 of the N-terminus relative to SEQ ID NOs: 71-73. In some embodiments, the transposase domain comprises a deletion of amino acids 1-103 of the N-terminus relative to SEQ ID NOs: 71-73.

Illustrative sequences of a PBx transposase domain comprising dual CRD domains with a deletion of amino acids 1-93 of the N-terminus are shown in SEQ ID NOs: 38 and 59:

(SEQ ID NO: 38)
NKHCWSTSKSTRRSRVSALNIVRSQRGPTRMCRNIYDPLLCFKLFFTDEIISEIVKWTNAE
ISLKRRESMTSATFRDTNEDEIYAFFGILVMTAVRKDNHMSTDDLFDRSLSMVYVSVMS
RDRFDFLIRCLRMDDKSIRPTLRENDVFTPVRKIWDLFIHQCIQNYTPGAHLTIDEQLLGF
RGRCPFRVYIPNKPSKYGIKILMMCDSGTKYMINGMPYLGRGTQTNGVPLGEYYVKELS
KPVHGSCRNITCDNWFTSIPLAKNLLQEPYKLTIVGTVASNAREIPEVLKNSRSRPVGTS
MFCFDGPLTLVSYKPKPAKMVYLLSSCDEDASINESTGKPQMVMYYNQTKGGVDTLN
QMCSVMTCSRKTNRWPMALLYGMINIACINSFIIYSHNVSSKGEKVQSRKKFMRNLYM
SLTSSFMRKRLEAPTLKRYLRDNISNILPKEVPGTSDDSTEEPVMKKRTYCTYCPSKIRRK
ANASCKKCKKVICREHNIDMCQSCFAGGGSTEEPVMKKRTYCTYCPSKIRRKANASCK
KCKKVICREHNIDMCQSCF
(SEQ ID NO: 59)
NKHCWSTSKSTRRSRVSALNIVRSQRGPTRMCRNIYDPLLCFKLFFTDEIISEIVKWTNAE
ISLKRRESMTSATFRDTNEDEIYAFFGILVMTAVRKDNHMSTDDLFDRSLSMVYVSVMS
RDRFDFLIRCLRMDDKSIRPTLRENDVFTPVRKIWDLFIHQCIQNYTPGAHLTIDEQLLGF
RGRCPFRVYIPNKPSKYGIKILMMCDSGTKYMINGMPYLGRGTQTNGVPLGEYYVKELS
KPVHGSCRNITCDNWFTSIPLAKNLLQEPYKLTIVGTVASNAREIPEVLKNSRSRPVGTS
MFCFDGPLTLVSYKPKPAKMVYLLSSCDEDASINESTGKPQMVMYYNQTKGGVDTLN
QMCSVMTCSRKTNRWPMALLYGMINIACINSFIIYSHNVSSKGEKVQSRKKFMRNLYM
SLTSSFMRKRLEAPTLKRYLRDNISNILPKEVPGTSDDSTEEPVMKKRTYCTYCPSKIRRK
ANASCKKCKKVICREHNIDMCQSCFGTSDDSTEEPVMKKRTYCTYCPSKIRRKANASCK
KCKKVICREHNIDMCQSCF

Illustrative sequences of PBx transposase domains comprising N-terminal deletions and a second piggyBac CRD domain appended via an AGGG linker are set forth in SEQ ID NOs: 28-48 in Table 1.

TABLE 1
Illustrative sequences of N-terminally deleted, Dual CRD PBx Domains

| Deletion | Sequence |
|---|---|
| PBx | TLPQRTIRGKNKHCWSTSKSTRRSRVSALNIVRSQRGPTRMCRNIYDPLL |
| Delta 83 | CFKLFFTDEIISEIVKWTNAEISLKRRESMTSATFRDTNEDEIYAFFGILVM |
| N- | TAVRKDNHMSTDDLFDRSLSMVYVSVMSRDRFDFLIRCLRMDDKSIRPT |
| Terminal | LRENDVFTPVRKIWDLFIHQCIQNYTPGAHLTIDEQLLGFRGRCPFRVYIP |
| Dual | NKPSKYGIKILMMCDSGTKYMINGMPYLGRGTQTNGVPLGEYYVKELS |
| CRD | KPVHGSCRNITCDNWFTSIPLAKNLLQEPYKLTIVGTVASNAREIPEVLK |
| | NSRSRPVGTSMFCFDGPLTLVSYKPKPAKMVYLLSSCDEDASINESTGKP |
| | QMVMYYNQTKGGVDTLNQMCSVMTCSRKTNRWPMALLYGMINIACIN |
| | SFIIYSHNVSSKGEKVQSRKKFMRNLYMSLTSSFMRKRLEAPTLKRYLR |
| | DNISNILPKEVPGTSDDSTEEPVMKKRTYCTYCPSKIRRKANASCKKCKK |
| | VICREHNIDMCQSCFAGGGSTEEPVMKKRTYCTYCPSKIRRKANASCKK |
| | CKKVICREHNIDMCQSCF (SEQ ID NO: 28) |
| PBx | LPQRTIRGKNKHCWSTSKSTRRSRVSALNIVRSQRGPTRMCRNIYDPLLC |
| Delta 84 | FKLFFTDEIISEIVKWTNAEISLKRRESMTSATFRDTNEDEIYAFFGILVMT |
| N- | AVRKDNHMSTDDLFDRSLSMVYVSVMSRDRFDFLIRCLRMDDKSIRPTL |
| Terminal | RENDVFTPVRKIWDLFIHQCIQNYTPGAHLTIDEQLLGFRGRCPFRVYIPN |
| Dual | KPSKYGIKILMMCDSGTKYMINGMPYLGRGTQTNGVPLGEYYVKELSK |
| CRD | PVHGSCRNITCDNWFTSIPLAKNLLQEPYKLTIVGTVASNAREIPEVLKNS |
| | RSRPVGTSMFCFDGPLTLVSYKPKPAKMVYLLSSCDEDASINESTGKPQ |
| | MVMYYNQTKGGVDTLNQMCSVMTCSRKTNRWPMALLYGMINIACINS |
| | FIIYSHNVSSKGEKVQSRKKFMRNLYMSLTSSFMRKRLEAPTLKRYLRD |
| | NISNILPKEVPGTSDDSTEEPVMKKRTYCTYCPSKIRRKANASCKKCKKV |
| | ICREHNIDMCQSCFAGGGSTEEPVMKKRTYCTYCPSKIRRKANASCKKC |
| | KKVICREHNIDMCQSCF (SEQ ID NO: 29) |
| PBx | PQRTIRGKNKHCWSTSKSTRRSRVSALNIVRSQRGPTRMCRNIYDPLLCF |
| Delta 85 | KLFFTDEIISEIVKWTNAEISLKRRESMTSATFRDTNEDEIYAFFGILVMTA |
| N- | VRKDNHMSTDDLFDRSLSMVYVSVMSRDRFDFLIRCLRMDDKSIRPTLR |
| Terminal | ENDVFTPVRKIWDLFIHQCIQNYTPGAHLTIDEQLLGFRGRCPFR VYIPNK |
| Dual | PSKYGIKILMMCDSGTKYMINGMPYLGRGTQTNGVPLGEYYVKELSKP |
| CRD | VHGSCRNITCDNWFTSIPLAKNLLQEPYKLTIVGTVASNAREIPEVLKNS |
| | RSRPVGTSMFCFDGPLTLVSYKPKPAKMVYLLSSCDEDASINESTGKPQ |
| | MVMYYNQTKGGVDTLNQMCSVMTCSRKTNRWPMALLYGMINIACINS |
| | FIIYSHNVSSKGEKVQSRKKFMRNLYMSLTSSFMRKRLEAPTLKRYLRD |
| | NISNILPKEVPGTSDDSTEEPVMKKRTYCTYCPSKIRRKANASCKKCKKV |
| | ICREHNIDMCQSCFAGGGSTEEPVMKKRTYCTYCPSKIRRKANASCKKC |
| | KKVICREHNIDMCQSCF (SEQ ID NO: 30) |
| PBx | QRTIRGKNKHCWSTSKSTRRSRVSALNIVRSQRGPTRMCRNIYDPLLCFK |
| Delta 86 | LFFTDEIISEIVKWTNAEISLKRRESMTSATFRDTNEDEIYAFFGILVMTAV |
| N- | RKDNHMSTDDLFDRSLSMVYVSVMSRDRFDFLIRCLRMDDKSIRPTLRE |
| Terminal | NDVFTPVRKIWDLFIHQCIQNYTPGAHLTIDEQLLGFRGRCPFRVYIPNKP |
| | SKYGIKILMMCDSGTKYMINGMPYLGRGTQTNGVPLGEYYVKELSKPV |
| | HGSCRNITCDNWFTSIPLAKNLLQEPYKLTIVGTVASNAREIPEVLKNSRS |
| | RPVGTSMFCFDGPLTLVSYKPKPAKMVYLLSSCDEDASINESTGKPQMV |
| | MYYNQTKGGVDTLNQMCSVMTCSRKTNRWPMALLYGMINIACINSFII |
| Dual | YSHNVSSKGEKVQSRKKFMRNLYMSLTSSFMRKRLEAPTLKRYLRDNIS |
| CRD | NILPKEVPGTSDDSTEEPVMKKRTYCTYCPSKIRRKANASCKKCKKVICR |
| | EHNIDMCQSCFAGGGSTEEPVMKKRTYCTYCPSKIRRKANASCKKCKK |
| | VICREHNIDMCQSCF (SEQ ID NO: 31). |
| PBx | RTIRGKNKHCWSTSKSTRRSRVSALNIVRSQRGPTRMCRNIYDPLLCFKL |
| Delta 87 | FFTDEIISEIVKWTNAEISLKRRESMTSATFRDTNEDEIYAFFGILVMTAV |
| N- | RKDNHMSTDDLFDRSLSMVYVSVMSRDRFDFLIRCLRMDDKSIRPTLRE |
| Terminal | NDVFTPVRKIWDLFIHQCIQNYTPGAHLTIDEQLLGFRGRCPFRVYIPNKP |
| Dual | SKYGIKILMMCDSGTKYMINGMPYLGRGTQTNGVPLGEYYVKELSKPV |
| CRD | HGSCRNITCDNWFTSIPLAKNLLQEPYKLTIVGTVASNAREIPEVLKNSRS |
| | RPVGTSMFCFDGPLTLVSYKPKPAKMVYLLSSCDEDASINESTGKPQMV |
| | MYYNQTKGGVDTLNQMCSVMTCSRKTNRWPMALLYGMINIACINSFII |
| | YSHNVSSKGEKVQSRKKFMRNLYMSLTSSFMRKRLEAPTLKRYLRDNIS |
| | NILPKEVPGTSDDSTEEPVMKKRTYCTYCPSKIRRKANASCKKCKKVICR |
| | EHNIDMCQSCFAGGGSTEEPVMKKRTYCTYCPSKIRRKANASCKKCKK |
| | VICREHNIDMCQSCF (SEQ ID NO: 32) |
| PBx | TIRGKNKHCWSTSKSTRRSRVSALNIVRSQRGPTRMCRNIYDPLLCFKLF |
| Delta 88 | FTDEIISEIVKWTNAEISLKRRESMTSATFRDTNEDEIYAFFGILVMTAVR |
| N- | KDNHMSTDDLFDRSLSMVYVSVMSRDRFDFLIRCLRMDDKSIRPTLREN |
| Terminal | DVFTPVRKIWDLFIHQCIQNYTPGAHLTIDEQLLGFRGRCPFRVYIPNKPS |
| Dual | KYGIKILMMCDSGTKYMINGMPYLGRGTQTNGVPLGEYYVKELSKPVH |
| CRD | GSCRNITCDNWFTSIPLAKNLLQEPYKLTIVGTVASNAREIPEVLKNSRSR |
| | PVGTSMFCFDGPLTLVSYKPKPAKMVYLLSSCDEDASINESTGKPQMVM |
| | YYNQTKGGVDTLNQMCSVMTCSRKTNRWPMALLYGMINIACINSFIIYS |
| | HNVSSKGEKVQSRKKFMRNLYMSLTSSFMRKRLEAPTLKRYLRDNISNI |
| | LPKEVPGTSDDSTEEPVMKKRTYCTYCPSKIRRKANASCKKCKKVICRE |
| | HNIDMCQSCFAGGGSTEEPVMKKRTYCTYCPSKIRRKANASCKKCKKVI |
| | CREHNIDMCQSCF (SEQ ID NO: 33) |
| PBx | IRGKNKHCWSTSKSTRRSRVSALNIVRSQRGPTRMCRNIYDPLLCFKLFF |
| Delta 89 | TDEIISEIVKWTNAEISLKRRESMTSATFRDTNEDEIYAFFGILVMTAVRK |
| N- | DNHMSTDDLFDRSLSMVYVSVMSRDRFDFLIRCLRMDDKSIRPTLREND |
| Terminal | VFTPVRKIWDLFIHQCIQNYTPGAHLTIDEQLLGFRGRCPFRVYIPNKPSK |
| Dual | YGIKILMMCDSGTKYMINGMPYLGRGTQTNGVPLGEYYVKELSKPVHG |
| CRD | SCRNITCDNWFTSIPLAKNLLQEPYKLTIVGTVASNAREIPEVLKNSRSRP |
| | VGTSMFCFDGPLTLVSYKPKPAKMVYLLSSCDEDASINESTGKPQMVM |
| | YYNQTKGGVDTLNQMCSVMTCSRKTNRWPMALLYGMINIACINSFIIYS |
| | HNVSSKGEKVQSRKKFMRNLYMSLTSSFMRKRLEAPTLKRYLRDNISNI |
| | LPKEVPGTSDDSTEEPVMKKRTYCTYCPSKIRRKANASCKKCKKVICRE |
| | HNIDMCQSCFAGGGSTEEPVMKKRTYCTYCPSKIRRKANASCKKCKKVI |
| | CREHNIDMCQSCF (SEQ ID NO: 34) |
| PBx | RGKNKHCWSTSKSTRRSRVSALNIVRSQRGPTRMCRNIYDPLLCFKLFFT |
| Delta 90 | DEIISEIVKWTNAEISLKRRESMTSATFRDTNEDEIYAFFGILVMTAVRKD |
| N- | NHMSTDDLFDRSLSMVYVSVMSRDRFDFLIRCLRMDDKSIRPTLRENDV |
| Terminal | FTPVRKIWDLFIHQCIQNYTPGAHLTIDEQLLGFRGRCPFRVYIPNKPSKY |
| Dual | GIKILMMCDSGTKYMINGMPYLGRGTQTNGVPLGEYYVKELSKPVHGS |
| CRD | CRNITCDNWFTSIPLAKNLLQEPYKLTIVGTVASNAREIPEVLKNSRSRPV |
| | GTSMFCFDGPLTLVSYKPKPAKMVYLLSSCDEDASINESTGKPQMVMY |
| | YNQTKGGVDTLNQMCSVMTCSRKTNRWPMALLYGMINIACINSFIIYSH |
| | NVSSKGEKVQSRKKFMRNLYMSLTSSFMRKRLEAPTLKRYLRDNISNIL |
| | PKEVPGTSDDSTEEPVMKKRTYCTYCPSKIRRKANASCKKCKKVICREH |
| | NIDMCQSCFAGGGSTEEPVMKKRTYCTYCPSKIRRKANASCKKCKKVIC |
| | REHNIDMCQSCF (SEQ ID NO: 35) |
| PBx | GKNKHCWSTSKSTRRSRVSALNIVRSQRGPTRMCRNIYDPLLCFKLFFTD |
| Delta 91 | EIISEIVKWTNAEISLKRRESMTSATFRDTNEDEIYAFFGILVMTAVRKDN |
| N- | HMSTDDLFDRSLSMVYVSVMSRDRFDFLIRCLRMDDKSIRPTLRENDVF |
| Terminal | TPVRKIWDLFIHQCIQNYTPGAHLTIDEQLLGFRGRCPFRVYIPNKPSKYG |
| Dual | IKILMMCDSGTKYMINGMPYLGRGTQTNGVPLGEYYVKELSKPVHGSC |
| CRD | RNITCDNWFTSIPLAKNLLQEPYKLTIVGTVASNAREIPEVLKNSRSRPVG |
| | TSMFCFDGPLTLVSYKPKPAKMVYLLSSCDEDASINESTGKPQMVMYY |
| | NQTKGGVDTLNQMCSVMTCSRKTNRWPMALLYGMINIACINSFIIYSHN |
| | VSSKGEKVQSRKKFMRNLYMSLTSSFMRKRLEAPTLKRYLRDNISNILP |
| | KEVPGTSDDSTEEPVMKKRTYCTYCPSKIRRKANASCKKCKKVICREHN |
| | IDMCQSCFAGGGSTEEPVMKKRTYCTYCPSKIRRKANASCKKCKKVICR |
| | EHNIDMCQSCF (SEQ ID NO: 36) |
| PBx | KNKHCWSTSKSTRRSRVSALNIVRSQRGPTRMCRNIYDPLLCFKLFFTDE |
| Delta 92 | IISEIVKWTNAEISLKRRESMTSATFRDTNEDEIYAFFGILVMTAVRKDNH |
| N- | MSTDDLFDRSLSMVYVSVMSRDRFDFLIRCLRMDDKSIRPTLRENDVFT |
| Terminal | PVRKIWDLFIHQCIQNYTPGAHLTIDEQLLGFRGRCPFRVYIPNKPSKYGI |
| Dual | KILMMCDSGTKYMINGMPYLGRGTQTNGVPLGEYYVKELSKPVHGSCR |
| CRD | NITCDNWFTSIPLAKNLLQEPYKLTIVGTVASNAREIPEVLKNSRSRPVGT |
| | SMFCFDGPLTLVSYKPKPAKMVYLLSSCDEDASINESTGKPQMVMYYN |
| | QTKGGVDTLNQMCSVMTCSRKTNRWPMALLYGMINIACINSFIIYSHNV |
| | SSKGEKVQSRKKFMRNLYMSLTSSFMRKRLEAPTLKRYLRDNISNILPKE |
| | VPGTSDDSTEEPVMKKRTYCTYCPSKIRRKANASCKKCKKVICREHNID |
| | MCQSCFAGGGSTEEPVMKKRTYCTYCPSKIRRKANASCKKCKKVICRE |
| | HNIDMCQSCF (SEQ ID NO: 37) |
| PBx | NKHCWSTSKSTRRSRVSALNIVRSQRGPTRMCRNIYDPLLCFKLFFTDEII |
| Delta 93 | SEIVKWTNAEISLKRRESMTSATFRDTNEDEIYAFFGILVMTAVRKDNH |
| N- | MSTDDLFDRSLSMVYVSVMSRDRFDFLIRCLRMDDKSIRPTLRENDVFT |
| Terminal | PVRKIWDLFIHQCIQNYTPGAHLTIDEQLLGFRGRCPFRVYIPNKPSKYGI |
| Dual | KILMMCDSGTKYMINGMPYLGRGTQTNGVPLGEYYVKELSKPVHGSCR |
| CRD | NITCDNWFTSIPLAKNLLQEPYKLTIVGTVASNAREIPEVLKNSRSRPVGT |
| | SMFCFDGPLTLVSYKPKPAKMVYLLSSCDEDASINESTGKPQMVMYYN |
| | QTKGGVDTLNQMCSVMTCSRKTNRWPMALLYGMINIACINSFIIYSHNV |
| | SSKGEKVQSRKKFMRNLYMSLTSSFMRKRLEAPTLKRYLRDNISNILPKE |
| | VPGTSDDSTEEPVMKKRTYCTYCPSKIRRKANASCKKCKKVICREHNID |
| | MCQSCFAGGGSTEEPVMKKRTYCTYCPSKIRRKANASCKKCKKVICRE |
| | HNIDMCQSCF (SEQ ID NO: 38) |
| PBx | KHCWSTSKSTRRSRVSALNIVRSQRGPTRMCRNIYDPLLCFKLFFTDEIIS |
| Delta 94 | EIVKWTNAEISLKRRESMTSATFRDTNEDEIYAFFGILVMTAVRKDNHM |
| N- | STDDLFDRSLSMVYVSVMSRDRFDFLIRCLRMDDKSIRPTLRENDVFTPV |
| Terminal | RKIWDLFIHQCIQNYTPGAHLTIDEQLLGFRGRCPFRVYIPNKPSKYGIKI |
| Dual | LMMCDSGTKYMINGMPYLGRGTQTNGVPLGEYYVKELSKPVHGSCRNI |
| CRD | TCDNWFTSIPLAKNLLQEPYKLTIVGTVASNAREIPEVLKNSRSRPVGTS |
| | MFCFDGPLTLVSYKPKPAKMVYLLSSCDEDASINESTGKPQMVMYYNQ |
| | TKGGVDTLNQMCSVMTCSRKTNRWPMALLYGMINIACINSFIIYSHNVS |
| | SKGEKVQSRKKFMRNLYMSLTSSFMRKRLEAPTLKRYLRDNISNILPKE |
| | VPGTSDDSTEEPVMKKRTYCTYCPSKIRRKANASCKKCKKVICREHNID |
| | MCQSCFAGGGSTEEPVMKKRTYCTYCPSKIRRKANASCKKCKKVICRE |
| | HNIDMCQSCF (SEQ ID NO: 39) |
| PBx | HCWSTSKSTRRSRVSALNIVRSQRGPTRMCRNIYDPLLCFKLFFTDEIISEI |
| Delta 95 | VKWTNAEISLKRRESMTSATFRDTNEDEIYAFFGILVMTAVRKDNHMST |
| N- | DDLFDRSLSMVYVSVMSRDRFDFLIRCLRMDDKSIRPTLRENDVFTPVR |
| Terminal | KIWDLFIHQCIQNYTPGAHLTIDEQLLGFRGRCPFRVYIPNKPSKYGIKIL |
| Dual | MMCDSGTKYMINGMPYLGRGTQTNGVPLGEYYVKELSKPVHGSCRNIT |
| CRD | CDNWFTSIPLAKNLLQEPYKLTIVGTVASNAREIPEVLKNSRSRPVGTSM |
| | FCFDGPLTLVSYKPKPAKMVYLLSSCDEDASINESTGKPQMVMYYNQT |
| | KGGVDTLNQMCSVMTCSRKTNRWPMALLYGMINIACINSFIIYSHNVSS |
| | KGEKVQSRKKFMRNLYMSLTSSFMRKRLEAPTLKRYLRDNISNILPKEV |
| | PGTSDDSTEEPVMKKRTYCTYCPSKIRRKANASCKKCKKVICREHNIDM |
| | CQSCFAGGGSTEEPVMKKRTYCTYCPSKIRRKANASCKKCKKVICREHN |
| | IDMCQSCF (SEQ ID NO: 40) |
| PBx | CWSTSKSTRRSRVSALNIVRSQRGPTRMCRNIYDPLLCFKLFFTDEIISEIV |
| Delta 96 | KWTNAEISLKRRESMTSATFRDTNEDEIYAFFGILVMTAVRKDNHMSTD |
| N- | DLFDRSLSMVYVSVMSRDRFDFLIRCLRMDDKSIRPTLRENDVFTPVRKI |
| Terminal | WDLFIHQCIQNYTPGAHLTIDEQLLGFRGRCPFRVYIPNKPSKYGIKILM |
| Dual | MCDSGTKYMINGMPYLGRGTQTNGVPLGEYYVKELSKPVHGSCRNITC |
| CRD | DNWFTSIPLAKNLLQEPYKLTIVGTVASNAREIPEVLKNSRSRPVGTSMF |
| | CFDGPLTLVSYKPKPAKMVYLLSSCDEDASINESTGKPQMVMYYNQTK |
| | GGVDTLNQMCSVMTCSRKTNRWPMALLYGMINIACINSFIIYSHNVSSK |
| | GEKVQSRKKFMRNLYMSLTSSFMRKRLEAPTLKRYLRDNISNILPKEVP |
| | GTSDDSTEEPVMKKRTYCTYCPSKIRRKANASCKKCKKVICREHNIDMC |
| | QSCFAGGGSTEEPVMKKRTYCTYCPSKIRRKANASCKKCKKVICREHNI |
| | DMCQSCF (SEQ ID NO: 41) |
| PBx | WSTSKSTRRSRVSALNIVRSQRGPTRMCRNIYDPLLCFKLFFTDEIISEIVK |
| Delta 97 | WTNAEISLKRRESMTSATFRDTNEDEIYAFFGILVMTAVRKDNHMSTDD |
| N- | LFDRSLSMVYVSVMSRDRFDFLIRCLRMDDKSIRPTLRENDVFTPVRKIW |
| Terminal | DLFIHQCIQNYTPGAHLTIDEQLLGFRGRCPFRVYIPNKPSKYGIKILMMC |
| Dual | DSGTKYMINGMPYLGRGTQTNGVPLGEYYVKELSKPVHGSCRNITCDN |
| CRD | WFTSIPLAKNLLQEPYKLTIVGTVASNAREIPEVLKNSRSRPVGTSMFCF |
| | DGPLTLVSYKPKPAKMVYLLSSCDEDASINESTGKPQMVMYYNQTKGG |
| | VDTLNQMCSVMTCSRKTNRWPMALLYGMINIACINSFIIYSHNVSSKGE |
| | KVQSRKKFMRNLYMSLTSSFMRKRLEAPTLKRYLRDNISNILPKEVPGTS |
| | DDSTEEPVMKKRTYCTYCPSKIRRKANASCKKCKKVICREHNIDMCQSC |
| | FAGGGSTEEPVMKKRTYCTYCPSKIRRKANASCKKCKKVICREHNIDMC |
| | QSCF (SEQ ID NO: 42) |
| PBx | STSKSTRRSRVSALNIVRSQRGPTRMCRNIYDPLLCFKLFFTDEIISEIVKW |
| Delta 98 | TNAEISLKRRESMTSATFRDTNEDEIYAFFGILVMTAVRKDNHMSTDDLF |
| N- | DRSLSMVYVSVMSRDRFDFLIRCLRMDDKSIRPTLRENDVFTPVRKIWD |
| Terminal | LFIHQCIQNYTPGAHLTIDEQLLGFRGRCPFRVYIPNKPSKYGIKILMMCD |
| Dual | SGTKYMINGMPYLGRGTQTNGVPLGEYYVKELSKPVHGSCRNITCDNW |
| CRD | FTSIPLAKNLLQEPYKLTIVGTVASNAREIPEVLKNSRSRPVGTSMFCFDG |
| | PLTLVSYKPKPAKMVYLLSSCDEDASINESTGKPQMVMYYNQTKGGVD |
| | TLNQMCSVMTCSRKTNRWPMALLYGMINIACINSFIIYSHNVSSKGEKV |
| | QSRKKFMRNLYMSLTSSFMRKRLEAPTLKRYLRDNISNILPKEVPGTSDD |
| | STEEPVMKKRTYCTYCPSKIRRKANASCKKCKKVICREHNIDMCQSCFA |
| | GGGSTEEPVMKKRTYCTYCPSKIRRKANASCKKCKKVICREHNIDMCQS |
| | CF (SEQ ID NO: 43) |
| PBx | TSKSTRRSRVSALNIVRSQRGPTRMCRNIYDPLLCFKLFFTDEIISEIVKWT |
| Delta 99 | NAEISLKRRESMTSATFRDTNEDEIYAFFGILVMTAVRKDNHMSTDDLF |
| N- | DRSLSMVYVSVMSRDRFDFLIRCLRMDDKSIRPTLRENDVFTPVRKIWD |
| Terminal | LFIHQCIQNYTPGAHLTIDEQLLGFRGRCPFRVYIPNKPSKYGIKILMMCD |
| Dual | SGTKYMINGMPYLGRGTQTNGVPLGEYYVKELSKPVHGSCRNITCDNW |
| CRD | FTSIPLAKNLLQEPYKLTIVGTVASNAREIPEVLKNSRSRPVGTSMFCFDG |
| | PLTLVSYKPKPAKMVYLLSSCDEDASINESTGKPQMVMYYNQTKGGVD |
| | TLNQMCSVMTCSRKTNRWPMALLYGMINIACINSFIIYSHNVSSKGEKV |
| | QSRKKFMRNLYMSLTSSFMRKRLEAPTLKRYLRDNISNILPKEVPGTSDD |
| | STEEPVMKKRTYCTYCPSKIRRKANASCKKCKKVICREHNIDMCQSCFA |
| | GGGSTEEPVMKKRTYCTYCPSKIRRKANASCKKCKKVICREHNIDMCQS |
| | CF (SEQ ID NO: 44) |
| PBx | SKSTRRSRVSALNIVRSQRGPTRMCRNIYDPLLCFKLFFTDEIISEIVKWT |
| Delta 100 | NAEISLKRRESMTSATFRDTNEDEIYAFFGILVMTAVRKDNHMSTDDLF |
| N- | DRSLSMVYVSVMSRDRFDFLIRCLRMDDKSIRPTLRENDVFTPVRKIWD |
| Terminal | LFIHQCIQNYTPGAHLTIDEQLLGFRGRCPFRVYIPNKPSKYGIKILMMCD |
| Dual | SGTKYMINGMPYLGRGTQTNGVPLGEYYVKELSKPVHGSCRNITCDNW |
| CRD | FTSIPLAKNLLQEPYKLTIVGTVASNAREIPEVLKNSRSRPVGTSMFCFDG |
| | PLTLVSYKPKPAKMVYLLSSCDEDASINESTGKPQMVMYYNQTKGGVD |
| | TLNQMCSVMTCSRKTNRWPMALLYGMINIACINSFIIYSHNVSSKGEKV |
| | QSRKKFMRNLYMSLTSSFMRKRLEAPTLKRYLRDNISNILPKEVPGTSDD |
| | STEEPVMKKRTYCTYCPSKIRRKANASCKKCKKVICREHNIDMCQSCFA |
| | GGGSTEEPVMKKRTYCTYCPSKIRRKANASCKKCKKVICREHNIDMCQS |
| | CF (SEQ ID NO: 45) |
| PBx | KSTRRSRVSALNIVRSQRGPTRMCRNIYDPLLCFKLFFTDEIISEIVKWTN |
| Delta 101 | AEISLKRRESMTSATFRDTNEDEIYAFFGILVMTAVRKDNHMSTDDLFD |
| N- | RSLSMVYVSVMSRDRFDFLIRCLRMDDKSIRPTLRENDVFTPVRKIWDLF |
| Terminal | IHQCIQNYTPGAHLTIDEQLLGFRGRCPFRVYIPNKPSKYGIKILMMCDSG |
| Dual | TKYMINGMPYLGRGTQTNGVPLGEYYVKELSKPVHGSCRNITCDNWFT |
| CRD | SIPLAKNLLQEPYKLTIVGTVASNAREIPEVLKNSRSRPVGTSMFCFDGPL |
| | TLVSYKPKPAKMVYLLSSCDEDASINESTGKPQMVMYYNQTKGGVDTL |
| | NQMCSVMTCSRKTNRWPMALLYGMINIACINSFIIYSHNVSSKGEKVQS |
| | RKKFMRNLYMSLTSSFMRKRLEAPTLKRYLRDNISNILPKEVPGTSDDST |
| | EEPVMKKRTYCTYCPSKIRRKANASCKKCKKVICREHNIDMCQSCFAGG |
| | GSTEEPVMKKRTYCTYCPSKIRRKANASCKKCKKVICREHNIDMCQSCF |
| | (SEQ ID NO: 46) |
| PBx | STRRSRVSALNIVRSQRGPTRMCRNIYDPLLCFKLFFTDEIISEIVKWTNA |
| Delta 102 | EISLKRRESMTSATFRDTNEDEIYAFFGILVMTAVRKDNHMSTDDLFDRS |
| N- | LSMVYVSVMSRDRFDFLIRCLRMDDKSIRPTLRENDVFTPVRKIWDLFIH |
| Terminal | QCIQNYTPGAHLTIDEQLLGFRGRCPFRVYIPNKPSKYGIKILMMCDSGT |
| Dual | KYMINGMPYLGRGTQTNGVPLGEYYVKELSKPVHGSCRNITCDNWFTSI |
| CRD | PLAKNLLQEPYKLTIVGTVASNAREIPEVLKNSRSRPVGTSMFCFDGPLT |
| | LVSYKPKPAKMVYLLSSCDEDASINESTGKPQMVMYYNQTKGGVDTLN |
| | QMCSVMTCSRKTNRWPMALLYGMINIACINSFIIYSHNVSSKGEKVQSR |
| | KKFMRNLYMSLTSSFMRKRLEAPTLKRYLRDNISNILPKEVPGTSDDSTE |
| | EPVMKKRTYCTYCPSKIRRKANASCKKCKKVICREHNIDMCQSCFAGG |
| | GSTEEPVMKKRTYCTYCPSKIRRKANASCKKCKKVICREHNIDMCQSCF |
| | (SEQ ID NO: 47) |
| PBx | TRRSRVSALNIVRSQRGPTRMCRNIYDPLLCFKLFFTDEIISEIVKWTNAEI |
| Delta 103 | SLKRRESMTSATFRDTNEDEIYAFFGILVMTAVRKDNHMSTDDLFDRSL |
| N- | SMVYVSVMSRDRFDFLIRCLRMDDKSIRPTLRENDVFTPVRKIWDLFIHQ |
| Terminal | CIQNYTPGAHLTIDEQLLGFRGRCPFRVYIPNKPSKYGIKILMMCDSGTK |
| Dual | YMINGMPYLGRGTQTNGVPLGEYYVKELSKPVHGSCRNITCDNWFTSIP |
| CRD | LAKNLLQEPYKLTIVGTVASNAREIPEVLKNSRSRPVGTSMFCFDGPLTL |
| | VSYKPKPAKMVYLLSSCDEDASINESTGKPQMVMYYNQTKGGVDTLN |
| | QMCSVMTCSRKTNRWPMALLYGMINIACINSFIIYSHNVSSKGEKVQSR |
| | KKFMRNLYMSLTSSFMRKRLEAPTLKRYLRDNISNILPKEVPGTSDDSTE |
| | EPVMKKRTYCTYCPSKIRRKANASCKKCKKVICREHNIDMCQSCFAGG |
| | GSTEEPVMKKRTYCTYCPSKIRRKANASCKKCKKVICREHNIDMCQSCF |
| | (SEQ ID NO: 48) |

Further illustrative sequences of PBx transposase domains comprising N-terminal deletions and a second piggyBac CRD domain comprising SEQ ID NO: 14 are set forth in SEQ ID NOs: 49-69 in Table 2.

TABLE 2
Illustrative sequences of N-terminally deleted, Dual CRD PBx Domains

| Deletion | Sequence |
|---|---|
| PBx | TLPQRTIRGKNKHCWSTSKSTRRSRVSALNIVRSQRGPTRMCRNIYDPLL |
| Delta 83 | CFKLFFTDEIISEIVKWTNAEISLKRRESMTSATFRDTNEDEIYAFFGILVM |
| N- | TAVRKDNHMSTDDLFDRSLSMVYVSVMSRDRFDFLIRCLRMDDKSIRPT |
| Terminal | LRENDVFTPVRKIWDLFIHQCIQNYTPGAHLTIDEQLLGFRGRCPFRVYIP |
| Dual | NKPSKYGIKILMMCDSGTKYMINGMPYLGRGTQTNGVPLGEYYVKELS |
| CRD | KPVHGSCRNITCDNWFTSIPLAKNLLQEPYKLTIVGTVASNAREIPEVLK |
| | NSRSRPVGTSMFCFDGPLTLVSYKPKPAKMVYLLSSCDEDASINESTGKP |
| | QMVMYYNQTKGGVDTLNQMCSVMTCSRKTNRWPMALLYGMINIACIN |
| | SFIIYSHNVSSKGEKVQSRKKFMRNLYMSLTSSFMRKRLEAPTLKRYLR |
| | DNISNILPKEVPGTSDDSTEEPVMKKRTYCTYCPSKIRRKANASCKKCKK |
| | VICREHNIDMCQSCFGTSDDSTEEPVMKKRTYCTYCPSKIRRKANASCK |
| | KCKKVICREHNIDMCQSCF (SEQ ID NO: 49) |
| PBx | LPQRTIRGKNKHCWSTSKSTRRSRVSALNIVRSQRGPTRMCRNIYDPLLC |
| Delta 84 | FKLFFTDEIISEIVKWTNAEISLKRRESMTSATFRDTNEDEIYAFFGILVMT |
| N- | AVRKDNHMSTDDLFDRSLSMVYVSVMSRDRFDFLIRCLRMDDKSIRPTL |
| Terminal | RENDVFTPVRKIWDLFIHQCIQNYTPGAHLTIDEQLLGFRGRCPFRVYIPN |
| Dual | KPSKYGIKILMMCDSGTKYMINGMPYLGRGTQTNGVPLGEYYVKELSK |
| CRD | PVHGSCRNITCDNWFTSIPLAKNLLQEPYKLTIVGTVASNAREIPEVLKNS |
| | RSRPVGTSMFCFDGPLTLVSYKPKPAKMVYLLSSCDEDASINESTGKPQ |
| | MVMYYNQTKGGVDTLNQMCSVMTCSRKTNRWPMALLYGMINIACINS |
| | FIIYSHNVSSKGEKVQSRKKFMRNLYMSLTSSFMRKRLEAPTLKRYLRD |
| | NISNILPKEVPGTSDDSTEEPVMKKRTYCTYCPSKIRRKANASCKKCKKV |
| | ICREHNIDMCQSCFGTSDDSTEEPVMKKRTYCTYCPSKIRRKANASCKK |
| | CKKVICREHNIDMCQSCF (SEQ ID NO: 50) |
| PBx | PQRTIRGKNKHCWSTSKSTRRSRVSALNIVRSQRGPTRMCRNIYDPLLCF |
| Delta 85 | KLFFTDEIISEIVKWTNAEISLKRRESMTSATFRDTNEDEIYAFFGILVMTA |
| N- | VRKDNHMSTDDLFDRSLSMVYVSVMSRDRFDFLIRCLRMDDKSIRPTLR |
| Terminal | ENDVFTPVRKIWDLFIHQCIQNYTPGAHLTIDEQLLGFRGRCPFRVYIPNK |
| Dual | PSKYGIKILMMCDSGTKYMINGMPYLGRGTQTNGVPLGEYYVKELSKP |
| CRD | VHGSCRNITCDNWFTSIPLAKNLLQEPYKLTIVGTVASNAREIPEVLKNS |
| | RSRPVGTSMFCFDGPLTLVSYKPKPAKMVYLLSSCDEDASINESTGKPQ |
| | MVMYYNQTKGGVDTLNQMCSVMTCSRKTNRWPMALLYGMINIACINS |
| | FIIYSHNVSSKGEKVQSRKKFMRNLYMSLTSSFMRKRLEAPTLKRYLRD |
| | NISNILPKEVPGTSDDSTEEPVMKKRTYCTYCPSKIRRKANASCKKCKKV |
| | ICREHNIDMCQSCFGTSDDSTEEPVMKKRTYCTYCPSKIRRKANASCKK |
| | CKKVICREHNIDMCQSCF (SEQ ID NO: 51) |
| PBx | QRTIRGKNKHCWSTSKSTRRSRVSALNIVRSQRGPTRMCRNIYDPLLCFK |
| Delta 86 | LFFTDEIISEIVKWTNAEISLKRRESMTSATFRDTNEDEIYAFFGILVMTAV |
| N- | RKDNHMSTDDLFDRSLSMVYVSVMSRDRFDFLIRCLRMDDKSIRPTLRE |
| Terminal | NDVFTPVRKIWDLFIHQCIQNYTPGAHLTIDEQLLGFRGRCPFRVYIPNKP |
| Dual | SKYGIKILMMCDSGTKYMINGMPYLGRGTQTNGVPLGEYYVKELSKPV |
| CRD | HGSCRNITCDNWFTSIPLAKNLLQEPYKLTIVGTVASNAREIPEVLKNSRS |
| | RPVGTSMFCFDGPLTLVSYKPKPAKMVYLLSSCDEDASINESTGKPQMV |
| | MYYNQTKGGVDTLNQMCSVMTCSRKTNRWPMALLYGMINIACINSFII |
| | YSHNVSSKGEKVQSRKKFMRNLYMSLTSSFMRKRLEAPTLKRYLRDNIS |
| | NILPKEVPGTSDDSTEEPVMKKRTYCTYCPSKIRRKANASCKKCKKVICR |
| | EHNIDMCQSCFGTSDDSTEEPVMKKRTYCTYCPSKIRRKANASCKKCKK |
| | VICREHNIDMCQSCF (SEQ ID NO: 52). |
| PBx | RTIRGKNKHCWSTSKSTRRSRVSALNIVRSQRGPTRMCRNIYDPLLCFKL |
| Delta 87 | FFTDEIISEIVKWTNAEISLKRRESMTSATFRDTNEDEIYAFFGILVMTAV |
| N- | RKDNHMSTDDLFDRSLSMVYVSVMSRDRFDFLIRCLRMDDKSIRPTLRE |
| Terminal | NDVFTPVRKIWDLFIHQCIQNYTPGAHLTIDEQLLGFRGRCPFRVYIPNKP |
| Dual | SKYGIKILMMCDSGTKYMINGMPYLGRGTQTNGVPLGEYYVKELSKPV |
| CRD | HGSCRNITCDNWFTSIPLAKNLLQEPYKLTIVGTVASNAREIPEVLKNSRS |
| | RPVGTSMFCFDGPLTLVSYKPKPAKMVYLLSSCDEDASINESTGKPQMV |
| | MYYNQTKGGVDTLNQMCSVMTCSRKTNRWPMALLYGMINIACINSFII |
| | YSHNVSSKGEKVQSRKKFMRNLYMSLTSSFMRKRLEAPTLKRYLRDNIS |
| | NILPKEVPGTSDDSTEEPVMKKRTYCTYCPSKIRRKANASCKKCKKVICR |
| | EHNIDMCQSCFGTSDDSTEEPVMKKRTYCTYCPSKIRRKANASCKKCKK |
| | VICREHNIDMCQSCF (SEQ ID NO: 53) |
| PBx | TIRGKNKHCWSTSKSTRRSRVSALNIVRSQRGPTRMCRNIYDPLLCFKLF |
| Delta 88 | FTDEIISEIVKWTNAEISLKRRESMTSATFRDTNEDEIYAFFGIL VMTAVR |
| N- | KDNHMSTDDLFDRSLSMVYVSVMSRDRFDFLIRCLRMDDKSIRPTLREN |
| Terminal | DVFTPVRKIWDLFIHQCIQNYTPGAHLTIDEQLLGFRGRCPFRVYIPNKPS |
| Dual | KYGIKILMMCDSGTKYMINGMPYLGRGTQTNGVPLGEYYVKELSKPVH |
| CRD | GSCRNITCDNWFTSIPLAKNLLQEPYKLTIVGTVASNAREIPEVLKNSRSR |
| | PVGTSMFCFDGPLTLVSYKPKPAKMVYLLSSCDEDASINESTGKPQMVM |
| | YYNQTKGGVDTLNQMCSVMTCSRKTNRWPMALLYGMINIACINSFIIYS |
| | HNVSSKGEKVQSRKKFMRNLYMSLTSSFMRKRLEAPTLKRYLRDNISNI |
| | LPKEVPGTSDDSTEEPVMKKRTYCTYCPSKIRRKANASCKKCKKVICRE |
| | HNIDMCQSCFGTSDDSTEEPVMKKRTYCTYCPSKIRRKANASCKKCKKV |
| | ICREHNIDMCQSCF (SEQ ID NO: 54) |
| PBx | IRGKNKHCWSTSKSTRRSRVSALNIVRSQRGPTRMCRNIYDPLLCFKLFF |
| Delta 89 | TDEIISEIVKWTNAEISLKRRESMTSATFRDTNEDEIYAFFGILVMTAVRK |
| N- | DNHMSTDDLFDRSLSMVYVSVMSRDRFDFLIRCLRMDDKSIRPTLREND |
| Terminal | VFTPVRKIWDLFIHQCIQNYTPGAHLTIDEQLLGFRGRCPFRVYIPNKPSK |
| Dual | YGIKILMMCDSGTKYMINGMPYLGRGTQTNGVPLGEYYVKELSKPVHG |
| CRD | SCRNITCDNWFTSIPLAKNLLQEPYKLTIVGTVASNAREIPEVLKNSRSRP |
| | VGTSMFCFDGPLTLVSYKPKPAKMVYLLSSCDEDASINESTGKPQMVM |
| | YYNQTKGGVDTLNQMCSVMTCSRKTNRWPMALLYGMINIACINSFIIYS |
| | HNVSSKGEKVQSRKKFMRNLYMSLTSSFMRKRLEAPTLKRYLRDNISNI |
| | LPKEVPGTSDDSTEEPVMKKRTYCTYCPSKIRRKANASCKKCKKVICRE |
| | HNIDMCQSCFGTSDDSTEEPVMKKRTYCTYCPSKIRRKANASCKKCKKV |
| | ICREHNIDMCQSCF (SEQ ID NO: 55) |
| PBx | RGKNKHCWSTSKSTRRSRVSALNIVRSQRGPTRMCRNIYDPLLCFKLFFT |
| Delta 90 | DEIISEIVKWTNAEISLKRRESMTSATFRDTNEDEIYAFFGILVMTAVRKD |
| N- | NHMSTDDLFDRSLSMVYVSVMSRDRFDFLIRCLRMDDKSIRPTLRENDV |
| Terminal | FTPVRKIWDLFIHQCIQNYTPGAHLTIDEQLLGFRGRCPFRVYIPNKPSKY |
| Dual | GIKILMMCDSGTKYMINGMPYLGRGTQTNGVPLGEYYVKELSKPVHGS |
| CRD | CRNITCDNWFTSIPLAKNLLQEPYKLTIVGTVASNAREIPEVLKNSRSRPV |
| | GTSMFCFDGPLTLVSYKPKPAKMVYLLSSCDEDASINESTGKPQMVMY |
| | YNQTKGGVDTLNQMCSVMTCSRKTNRWPMALLYGMINIACINSFIIYSH |
| | NVSSKGEKVQSRKKFMRNLYMSLTSSFMRKRLEAPTLKRYLRDNISNIL |
| | PKEVPGTSDDSTEEPVMKKRTYCTYCPSKIRRKANASCKKCKKVICREH |
| | NIDMCQSCFGTSDDSTEEPVMKKRTYCTYCPSKIRRKANASCKKCKKVI |
| | CREHNIDMCQSCF (SEQ ID NO: 56) |
| PBx | GKNKHCWSTSKSTRRSRVSALNIVRSQRGPTRMCRNIYDPLLCFKLFFTD |
| Delta 91 | EIISEIVKWTNAEISLKRRESMTSATFRDTNEDEIYAFFGILVMTAVRKDN |
| N- | HMSTDDLFDRSLSMVYVSVMSRDRFDFLIRCLRMDDKSIRPTLRENDVF |
| Terminal | TPVRKIWDLFIHQCIQNYTPGAHLTIDEQLLGFRGRCPFRVYIPNKPSKYG |
| Dual | IKILMMCDSGTKYMINGMPYLGRGTQTNGVPLGEYYVKELSKPVHGSC |
| CRD | RNITCDNWFTSIPLAKNLLQEPYKLTIVGTVASNAREIPEVLKNSRSRPVG |
| | TSMFCFDGPLTLVSYKPKPAKMVYLLSSCDEDASINESTGKPQMVMYY |
| | NQTKGGVDTLNQMCSVMTCSRKTNRWPMALLYGMINIACINSFIIYSHN |
| | VSSKGEKVQSRKKFMRNLYMSLTSSFMRKRLEAPTLKRYLRDNISNILP |
| | KEVPGTSDDSTEEPVMKKRTYCTYCPSKIRRKANASCKKCKKVICREHN |
| | IDMCQSCFGTSDDSTEEPVMKKRTYCTYCPSKIRRKANASCKKCKKVIC |
| | REHNIDMCQSCF (SEQ ID NO: 57) |
| PBx | KNKHCWSTSKSTRRSRVSALNIVRSQRGPTRMCRNIYDPLLCFKLFFTDE |
| Delta 92 | IISEIVKWTNAEISLKRRESMTSATFRDTNEDEIYAFFGILVMTAVRKDNH |
| N- | MSTDDLFDRSLSMVYVSVMSRDRFDFLIRCLRMDDKSIRPTLRENDVFT |
| Terminal | PVRKIWDLFIHQCIQNYTPGAHLTIDEQLLGFRGRCPFRVYIPNKPSKYGI |
| Dual | KILMMCDSGTKYMINGMPYLGRGTQTNGVPLGEYYVKELSKPVHGSCR |
| CRD | NITCDNWFTSIPLAKNLLQEPYKLTIVGTVASNAREIPEVLKNSRSRPVGT |
| | SMFCFDGPLTLVSYKPKPAKMVYLLSSCDEDASINESTGKPQMVMYYN |
| | QTKGGVDTLNQMCSVMTCSRKTNRWPMALLYGMINIACINSFIIYSHNV |
| | SSKGEKVQSRKKFMRNLYMSLTSSFMRKRLEAPTLKRYLRDNISNILPKE |
| | VPGTSDDSTEEPVMKKRTYCTYCPSKIRRKANASCKKCKKVICREHNID |
| | MCQSCFGTSDDSTEEPVMKKRTYCTYCPSKIRRKANASCKKCKKVICRE |
| | HNIDMCQSCF (SEQ ID NO: 58) |
| PBx | NKHCWSTSKSTRRSRVSALNIVRSQRGPTRMCRNIYDPLLCFKLFFTDEII |
| Delta 93 | SEIVKWTNAEISLKRRESMTSATFRDTNEDEIYAFFGILVMTAVRKDNH |
| N- | MSTDDLFDRSLSMVYVSVMSRDRFDFLIRCLRMDDKSIRPTLRENDVFT |
| Terminal | PVRKIWDLFIHQCIQNYTPGAHLTIDEQLLGFRGRCPFRVYIPNKPSKYGI |
| Dual | KILMMCDSGTKYMINGMPYLGRGTQTNGVPLGEYYVKELSKPVHGSCR |
| CRD | NITCDNWFTSIPLAKNLLQEPYKLTIVGTVASNAREIPEVLKNSRSRPVGT |
| | SMFCFDGPLTLVSYKPKPAKMVYLLSSCDEDASINESTGKPQMVMYYN |
| | QTKGGVDTLNQMCSVMTCSRKTNRWPMALLYGMINIACINSFIIYSHNV |
| | SSKGEKVQSRKKFMRNLYMSLTSSFMRKRLEAPTLKRYLRDNISNILPKE |
| | VPGTSDDSTEEPVMKKRTYCTYCPSKIRRKANASCKKCKKVICREHNID |
| | MCQSCFGTSDDSTEEPVMKKRTYCTYCPSKIRRKANASCKKCKKVICRE |
| | HNIDMCQSCF (SEQ ID NO: 59) |
| PBx | KHCWSTSKSTRRSRVSALNIVRSQRGPTRMCRNIYDPLLCFKLFFTDEIIS |
| Delta 94 | EIVKWTNAEISLKRRESMTSATFRDTNEDEIYAFFGILVMTAVRKDNHM |
| N- | STDDLFDRSLSMVYVSVMSRDRFDFLIRCLRMDDKSIRPTLRENDVFTPV |
| Terminal | RKIWDLFIHQCIQNYTPGAHLTIDEQLLGFRGRCPFRVYIPNKPSKYGIKI |
| Dual | LMMCDSGTKYMINGMPYLGRGTQTNGVPLGEYYVKELSKPVHGSCRNI |
| CRD | TCDNWFTSIPLAKNLLQEPYKLTIVGTVASNAREIPEVLKNSRSRPVGTS |
| | MFCFDGPLTLVSYKPKPAKMVYLLSSCDEDASINESTGKPQMVMYYNQ |
| | TKGGVDTLNQMCSVMTCSRKTNRWPMALLYGMINIACINSFIIYSHNVS |
| | SKGEKVQSRKKFMRNLYMSLTSSFMRKRLEAPTLKRYLRDNISNILPKE |
| | VPGTSDDSTEEPVMKKRTYCTYCPSKIRRKANASCKKCKKVICREHNID |
| | MCQSCFGTSDDSTEEPVMKKRTYCTYCPSKIRRKANASCKKCKKVICRE |
| | HNIDMCQSCF (SEQ ID NO: 60) |
| PBx | HCWSTSKSTRRSRVSALNIVRSQRGPTRMCRNIYDPLLCFKLFFTDEIISEI |
| Delta 95 | VKWTNAEISLKRRESMTSATFRDTNEDEIYAFFGILVMTAVRKDNHMST |
| N- | DDLFDRSLSMVYVSVMSRDRFDFLIRCLRMDDKSIRPTLRENDVFTPVR |
| Terminal | KIWDLFIHQCIQNYTPGAHLTIDEQLLGFRGRCPFRVYIPNKPSKYGIKIL |
| Dual | MMCDSGTKYMINGMPYLGRGTQTNGVPLGEYYVKELSKPVHGSCRNIT |
| CRD | CDNWFTSIPLAKNLLQEPYKLTIVGTVASNAREIPEVLKNSRSRPVGTSM |
| | FCFDGPLTLVSYKPKPAKMVYLLSSCDEDASINESTGKPQMVMYYNQT |
| | KGGVDTLNQMCSVMTCSRKTNRWPMALLYGMINIACINSFIIYSHNVSS |
| | KGEKVQSRKKFMRNLYMSLTSSFMRKRLEAPTLKRYLRDNISNILPKEV |
| | PGTSDDSTEEPVMKKRTYCTYCPSKIRRKANASCKKCKKVICREHNIDM |
| | CQSCFGTSDDSTEEPVMKKRTYCTYCPSKIRRKANASCKKCKKVICREH |
| | NIDMCQSCF (SEQ ID NO: 61) |
| PBx | CWSTSKSTRRSRVSALNIVRSQRGPTRMCRNIYDPLLCFKLFFTDEIISEIV |
| Delta 96 | KWTNAEISLKRRESMTSATFRDTNEDEIYAFFGILVMTAVRKDNHMSTD |
| N- | DLFDRSLSMVYVSVMSRDRFDFLIRCLRMDDKSIRPTLRENDVFTPVRKI |
| Terminal | WDLFIHQCIQNYTPGAHLTIDEQLLGFRGRCPFRVYIPNKPSKYGIKILM |
| Dual | MCDSGTKYMINGMPYLGRGTQTNGVPLGEYYVKELSKPVHGSCRNITC |
| CRD | DNWFTSIPLAKNLLQEPYKLTIVGTVASNAREIPEVLKNSRSRPVGTSMF |
| | CFDGPLTLVSYKPKPAKMVYLLSSCDEDASINESTGKPQMVMYYNQTK |
| | GGVDTLNQMCSVMTCSRKTNRWPMALLYGMINIACINSFIIYSHNVSSK |
| | GEKVQSRKKFMRNLYMSLTSSFMRKRLEAPTLKRYLRDNISNILPKEVP |
| | GTSDDSTEEPVMKKRTYCTYCPSKIRRKANASCKKCKKVICREHNIDMC |
| | QSCFGTSDDSTEEPVMKKRTYCTYCPSKIRRKANASCKKCKKVICREHNI |
| | DMCQSCF (SEQ ID NO: 62) |
| PBx | WSTSKSTRRSRVSALNIVRSQRGPTRMCRNIYDPLLCFKLFFTDEIISEIVK |
| Delta 97 | WTNAEISLKRRESMTSATFRDTNEDEIYAFFGILVMTAVRKDNHMSTDD |
| N- | LFDRSLSMVYVSVMSRDRFDFLIRCLRMDDKSIRPTLRENDVFTPVRKIW |
| Terminal | DLFIHQCIQNYTPGAHLTIDEQLLGFRGRCPFRVYIPNKPSKYGIKILMMC |
| Dual | DSGTKYMINGMPYLGRGTQTNGVPLGEYYVKELSKPVHGSCRNITCDN |
| CRD | WFTSIPLAKNLLQEPYKLTIVGTVASNAREIPEVLKNSRSRPVGTSMFCF |
| | DGPLTLVSYKPKPAKMVYLLSSCDEDASINESTGKPQMVMYYNQTKGG |
| | VDTLNQMCSVMTCSRKTNRWPMALLYGMINIACINSFIIYSHNVSSKGE |
| | KVQSRKKFMRNLYMSLTSSFMRKRLEAPTLKRYLRDNISNILPKEVPGTS |
| | DDSTEEPVMKKRTYCTYCPSKIRRKANASCKKCKKVICREHNIDMCQSC |
| | FGTSDDSTEEPVMKKRTYCTYCPSKIRRKANASCKKCKKVICREHNIDM |
| | CQSCF (SEQ ID NO: 63) |
| PBx | STSKSTRRSRVSALNIVRSQRGPTRMCRNIYDPLLCFKLFFTDEIISEIVKW |
| Delta 98 | TNAEISLKRRESMTSATFRDTNEDEIYAFFGILVMTAVRKDNHMSTDDLF |
| N- | DRSLSMVYVSVMSRDRFDFLIRCLRMDDKSIRPTLRENDVFTPVRKIWD |
| Terminal | LFIHQCIQNYTPGAHLTIDEQLLGFRGRCPFRVYIPNKPSKYGIKILMMCD |
| Dual | SGTKYMINGMPYLGRGTQTNGVPLGEYYVKELSKPVHGSCRNITCDNW |
| CRD | FTSIPLAKNLLQEPYKLTIVGTVASNAREIPEVLKNSRSRPVGTSMFCFDG |
| | PLTLVSYKPKPAKMVYLLSSCDEDASINESTGKPQMVMYYNQTKGGVD |
| | TLNQMCSVMTCSRKTNRWPMALLYGMINIACINSFIIYSHNVSSKGEKV |
| | QSRKKFMRNLYMSLTSSFMRKRLEAPTLKRYLRDNISNILPKEVPGTSDD |
| | STEEPVMKKRTYCTYCPSKIRRKANASCKKCKKVICREHNIDMCQSCFG |
| | TSDDSTEEPVMKKRTYCTYCPSKIRRKANASCKKCKKVICREHNIDMCQ |
| | SCF (SEQ ID NO: 64) |
| PBx | TSKSTRRSRVSALNIVRSQRGPTRMCRNIYDPLLCFKLFFTDEIISEIVKWT |
| Delta 99 | NAEISLKRRESMTSATFRDTNEDEIYAFFGILVMTAVRKDNHMSTDDLF |
| N- | DRSLSMVYVSVMSRDRFDFLIRCLRMDDKSIRPTLRENDVFTPVRKIWD |
| Terminal | LFIHQCIQNYTPGAHLTIDEQLLGFRGRCPFRVYIPNKPSKYGIKILMMCD |
| Dual | SGTKYMINGMPYLGRGTQTNGVPLGEYYVKELSKPVHGSCRNITCDNW |
| CRD | FTSIPLAKNLLQEPYKLTIVGTVASNAREIPEVLKNSRSRPVGTSMFCFDG |
| | PLTLVSYKPKPAKMVYLLSSCDEDASINESTGKPQMVMYYNQTKGGVD |
| | TLNQMCSVMTCSRKTNRWPMALLYGMINIACINSFIIYSHNVSSKGEKV |
| | QSRKKFMRNLYMSLTSSFMRKRLEAPTLKRYLRDNISNILPKEVPGTSDD |
| | STEEPVMKKRTYCTYCPSKIRRKANASCKKCKKVICREHNIDMCQSCFG |
| | TSDDSTEEPVMKKRTYCTYCPSKIRRKANASCKKCKKVICREHNIDMCQ |
| | SCF (SEQ ID NO: 65) |
| PBx | SKSTRRSRVSALNIVRSQRGPTRMCRNIYDPLLCFKLFFTDEIISEIVKWT |
| Delta 100 | NAEISLKRRESMTSATFRDTNEDEIYAFFGILVMTAVRKDNHMSTDDLF |
| N- | DRSLSMVYVSVMSRDRFDFLIRCLRMDDKSIRPTLRENDVFTPVRKIWD |
| Terminal | LFIHQCIQNYTPGAHLTIDEQLLGFRGRCPFRVYIPNKPSKYGIKILMMCD |
| Dual | SGTKYMINGMPYLGRGTQTNGVPLGEYYVKELSKPVHGSCRNITCDNW |
| CRD | FTSIPLAKNLLQEPYKLTIVGTVASNAREIPEVLKNSRSRPVGTSMFCFDG |
| | PLTLVSYKPKPAKMVYLLSSCDEDASINESTGKPQMVMYYNQTKGGVD |
| | TLNQMCSVMTCSRKTNRWPMALLYGMINIACINSFIIYSHNVSSKGEKV |
| | QSRKKFMRNLYMSLTSSFMRKRLEAPTLKRYLRDNISNILPKEVPGTSDD |
| | STEEPVMKKRTYCTYCPSKIRRKANASCKKCKKVICREHNIDMCQSCFG |
| | TSDDSTEEPVMKKRTYCTYCPSKIRRKANASCKKCKKVICREHNIDMCQ |
| | SCF (SEQ ID NO: 66) |
| PBx | KSTRRSRVSALNIVRSQRGPTRMCRNIYDPLLCFKLFFTDEIISEIVKWTN |
| Delta 101 | AEISLKRRESMTSATFRDTNEDEIYAFFGILVMTAVRKDNHMSTDDLFD |
| N- | RSLSMVYVSVMSRDRFDFLIRCLRMDDKSIRPTLRENDVFTPVRKIWDLF |
| Terminal | IHQCIQNYTPGAHLTIDEQLLGFRGRCPFRVYIPNKPSKYGIKILMMCDSG |
| Dual | TKYMINGMPYLGRGTQTNGVPLGEYYVKELSKPVHGSCRNITCDNWFT |
| CRD | SIPLAKNLLQEPYKLTIVGTVASNAREIPEVLKNSRSRPVGTSMFCFDGPL |
| | TLVSYKPKPAKMVYLLSSCDEDASINESTGKPQMVMYYNQTKGGVDTL |
| | NQMCSVMTCSRKTNRWPMALLYGMINIACINSFIIYSHNVSSKGEKVQS |
| | RKKFMRNLYMSLTSSFMRKRLEAPTLKRYLRDNISNILPKEVPGTSDDST |
| | EEPVMKKRTYCTYCPSKIRRKANASCKKCKKVICREHNIDMCQSCFGTS |
| | DDSTEEPVMKKRTYCTYCPSKIRRKANASCKKCKKVICREHNIDMCQSC |
| | F (SEQ ID NO: 67) |
| PBx | STRRSRVSALNIVRSQRGPTRMCRNIYDPLLCFKLFFTDEIISEIVKWTNA |
| Delta 102 | EISLKRRESMTSATFRDTNEDEIYAFFGILVMTAVRKDNHMSTDDLFDRS |
| N- | LSMVYVSVMSRDRFDFLIRCLRMDDKSIRPTLRENDVFTPVRKIWDLFIH |
| Terminal | QCIQNYTPGAHLTIDEQLLGFRGRCPFRVYIPNKPSKYGIKILMMCDSGT |
| Dual | KYMINGMPYLGRGTQTNGVPLGEYYVKELSKPVHGSCRNITCDNWFTSI |
| CRD | PLAKNLLQEPYKLTIVGTVASNAREIPEVLKNSRSRPVGTSMFCFDGPLT |
| | LVSYKPKPAKMVYLLSSCDEDASINESTGKPQMVMYYNQTKGGVDTLN |
| | QMCSVMTCSRKTNRWPMALLYGMINIACINSFIIYSHNVSSKGEKVQSR |
| | KKFMRNLYMSLTSSFMRKRLEAPTLKRYLRDNISNILPKEVPGTSDDSTE |
| | EPVMKKRTYCTYCPSKIRRKANASCKKCKKVICREHNIDMCQSCFGTSD |
| | DSTEEPVMKKRTYCTYCPSKIRRKANASCKKCKKVICREHNIDMCQSCF |
| | (SEQ ID NO: 68) |
| PBx | TRRSRVSALNIVRSQRGPTRMCRNIYDPLLCFKLFFTDEIISEIVKWTNAEI |
| Delta 103 | SLKRRESMTSATFRDTNEDEIYAFFGILVMTAVRKDNHMSTDDLFDRSL |
| N- | SMVYVSVMSRDRFDFLIRCLRMDDKSIRPTLRENDVFTPVRKIWDLFIHQ |
| Terminal | CIQNYTPGAHLTIDEQLLGFRGRCPFRVYIPNKPSKYGIKILMMCDSGTK |
| Dual | YMINGMPYLGRGTQTNGVPLGEYYVKELSKPVHGSCRNITCDNWFTSIP |
| CRD | LAKNLLQEPYKLTIVGTVASNAREIPEVLKNSRSRPVGTSMFCFDGPLTL |
| | VSYKPKPAKMVYLLSSCDEDASINESTGKPQMVMYYNQTKGGVDTLN |
| | QMCSVMTCSRKTNRWPMALLYGMINIACINSFIIYSHNVSSKGEKVQSR |
| | KKFMRNLYMSLTSSFMRKRLEAPTLKRYLRDNISNILPKEVPGTSDDSTE |
| | EPVMKKRTYCTYCPSKIRRKANASCKKCKKVICREHNIDMCQSCFGTSD |
| | DSTEEPVMKKRTYCTYCPSKIRRKANASCKKCKKVICREHNIDMCQSCF |
| | (SEQ ID NO: 69) |

Transposase Domains Comprising Thermostability and/or Hyperactivity Mutations

### Hyperactivity Mutations

The transposase domains provided herein may comprise one or more hyperactivity mutations.

In some embodiments, an SPB transposase domain provided herein comprises one or more hyperactivity mutations (in additional to those present in SPB). In some embodiments, an SPB transposase domain provided herein comprises a S103P mutation. In some embodiments, an SPB transposase domain provided herein comprises a R372H mutation. In some embodiments, an SPB transposase domain provided herein comprises a S509G mutation. In some embodiments, an SPB transposase domain provided herein comprises a N571S mutation. In some embodiments, an SPB transposase domain provided herein comprises a S103P mutation, a R372H mutation, a S509G mutation and a N571S mutation.

In some embodiments, a PBx transposase domain provided herein comprises one or more hyperactivity mutations (in additional to those present in PBx). In some embodiments, a PBx transposase domain provided herein comprises a S103P mutation. In some embodiments, a PBx transposase domain provided herein comprises a R372H mutation. In some embodiments, a PBx transposase domain provided herein comprises a S509G mutation. In some embodiments, a PBx transposase domain provided herein comprises a N571S mutation. In some embodiments, a PBx transposase domain provided herein comprises a S103P mutation, a R372H mutation, a S509G mutation, and a N571S mutation. In some embodiments, a PBx transposase domain comprising the R372H mutation comprises the amino acid sequence set forth in SEQ ID NO: 133. In some embodiments, a PBx transposase domain comprising the S103P, S509G, and N571S mutations comprises the amino acid sequence set forth in SEQ ID NO: 134. In some embodiments, a PBx transposase domain comprising the S103P, S509G, N571S, and R372H mutations comprises the amino acid sequence set forth in SEQ ID NO: 135.

The S103P, S509G, R372H and/or N571S mutation can also be introduced into any of the truncated SPBs or PBxs (e.g., a transposase domain comprising any one of SEQ ID NOs: 28-69) provided herein. A person of skill will appreciate that the numbering of the residues depends on the size of the truncation.

The S103P, S509G, R372H and/or N571S mutation can also be introduced into any of the transposase domains comprising dual CRDs provided herein. For example, an SPB transposase domain comprising the sequence set forth in SEQ ID NO: 1, 3 or 73 may further comprise a S103P, S509G, R372H and/or N571S mutation.

Similarly, a PBx transposase domain comprising the sequence set forth in any one of SEQ ID NO: 70-72 may further comprise a S103P, R372H S509G and/or N571S mutation. In some embodiments, a PBx domain comprising dual CRDs and an R372H mutation comprises the amino acid sequence set forth in SEQ ID NO: 138. In some embodiments, a PBx domain comprising dual CRDs and a S103P, S509G, and N571S mutation comprises the amino acid sequence set forth in SEQ ID NO: 139. In some embodiments, a PBx domain comprising dual CRDs and a S103P, S509G, N571S, and R372H mutation comprises the amino acid sequence set forth in SEQ ID NO: 140.

Similarly, the S103P, S509G, R372H and/or N571S mutation may be introduced into any of the fusion proteins described below. For example, a fusion protein comprising a transposase domain and a DNA binding domain may further comprise a S103P, S509G, R372H and/or N571S mutation.

### Thermostability Mutations

The transposase domains provided herein may comprise one or more thermostability mutations.

In some embodiments, an SPB transposase domain provided herein comprises one or more thermostability mutations. In some embodiments, an SPB transposase domain provided herein comprises one or more of the following mutations: I182L, S301A, C420M, M185L, R315K, D421H, F200W, Q318G, N427D, V207I, E331R, Q434E, M226F, V336I, V436I, I231T, S373K, I474L, V240K, V381E, K500R, Q254N, T392S, S513P, A263E, A411N, K525P, S289A, S419T, R567D, M298L, or any subgroup thereof. In some embodiments, an SPB transposase domain provided herein comprises a I182L mutation. In some embodiments, an SPB transposase domain provided herein comprises a S301A mutation. In some embodiments, an SPB transposase domain provided herein comprises a C420M mutation. In some embodiments, an SPB transposase domain provided herein comprises a M185L mutations. In some embodiments, an SPB transposase domain provided herein comprises a R315K mutation. In some embodiments, an SPB transposase domain provided herein comprises a D421H mutation.

In some embodiments, an SPB transposase domain provided herein comprises a F200W mutation, In some embodiments, an SPB transposase domain provided herein comprises a Q318G mutation. In some embodiments, an SPB transposase domain provided herein comprises a N427D mutation. In some embodiments, an SPB transposase domain provided herein comprises a V207I mutation. In some embodiments, an SPB transposase domain provided herein comprises a E331R mutation. In some embodiments, an SPB transposase domain provided herein comprises a Q434E mutation. In some embodiments, an SPB transposase domain provided herein comprises a M226F mutation. In some embodiments, an SPB transposase domain provided herein comprises a V336I, mutations. In some embodiments, an SPB transposase domain provided herein comprises a V436I mutation. In some embodiments, an SPB transposase domain provided herein comprises a I231T mutation. In some embodiments, an SPB transposase domain provided herein comprises a S373K mutation. In some embodiments, an SPB transposase domain provided herein comprises a I474L mutation. In some embodiments, an SPB transposase domain provided herein comprises a V240K mutation. In some embodiments, an SPB transposase domain provided herein comprises a V381E mutation. In some embodiments, an SPB transposase domain provided herein comprises a K500R mutation. In some embodiments, an SPB transposase domain provided herein comprises a Q254N mutation. In some embodiments, an SPB transposase domain provided herein comprises a T392S mutation. In some embodiments, an SPB transposase domain provided herein comprises a S513P mutation. In some embodiments, an SPB transposase domain provided herein comprises a A263E mutation. In some embodiments, an SPB transposase domain provided herein comprises a A411N mutation. In some embodiments, an SPB transposase domain provided herein comprises a K525P mutation. In some embodiments, an SPB transposase domain provided herein comprises a S289A mutation. In some embodiments, an SPB transposase domain provided herein comprises a S419T mutation. In some embodiments, an SPB transposase domain provided herein comprises a R567D mutation. In some embodiments, an SPB transposase domain provided herein comprises a M298L mutation.

In some embodiments, a PBx transposase domain provided herein comprises one or more thermostability mutations. In some embodiments, a PBx transposase domain provided herein comprises one or more of the following mutations: I182L, S301A, C420M, M185L, R315K, D421H, F200W, Q318G, N427D, V207I, E331R, Q434E, M226F, V336I, V436I, I231T, S373K, I474L, V240K, V381E, K500R, Q254N, T392S, S513P, A263E, A411N, K525P, S289A, S419T, R567D, M298L, or any subgroup thereof. In some embodiments, a PBx transposase domain provided herein comprises a I182L mutation. In some embodiments, a PBx transposase domain provided herein comprises a S301A mutation. In some embodiments, a PBx transposase domain provided herein comprises a C420M mutation. In some embodiments, a PBx transposase domain provided herein comprises a M185L mutations. In some embodiments, a PBx transposase domain provided herein comprises a R315K mutation. In some embodiments, a PBx transposase domain provided herein comprises a D421H mutation. In some embodiments, a PBx transposase domain provided herein comprises a F200W mutation, In some embodiments, a PBx transposase domain provided herein comprises a Q318G mutation. In some embodiments, a PBx transposase domain provided herein comprises a N427D mutation. In some embodiments, a PBx transposase domain provided herein comprises a V207I mutation. In some embodiments, a PBx transposase domain provided herein comprises a E331R mutation. In some embodiments, a PBx transposase domain provided herein comprises a Q434E mutation. In some embodiments, a PBx transposase domain provided herein comprises a M226F mutation. In some embodiments, a PBx transposase domain provided herein comprises a V336I, mutations. In some embodiments, a PBx transposase domain provided herein comprises a V436I mutation. In some embodiments, a PBx transposase domain provided herein comprises a I231T mutation. In some embodiments, a PBx transposase domain provided herein comprises a S373K mutation. In some embodiments, a PBx transposase domain provided herein comprises a I474L mutation. In some embodiments, a PBx transposase domain provided herein comprises a V240K mutation. In some embodiments, a PBx transposase domain provided herein comprises a V381E mutation. In some embodiments, a PBx transposase domain provided herein comprises a K500R mutation. In some embodiments, a PBx transposase domain provided herein comprises a Q254N mutation. In some embodiments, a PBx transposase domain provided herein comprises a T392S mutation. In some embodiments, a PBx transposase domain provided herein comprises a S513P mutation. In some embodiments, a PBx transposase domain provided herein comprises a A263E mutation. In some embodiments, a PBx transposase domain provided herein comprises a A411N mutation. In some embodiments, a PBx transposase domain provided herein comprises a K525P mutation. In some embodiments, a PBx transposase domain provided herein comprises a S289A mutation. In some embodiments, a PBx transposase domain provided herein comprises a S419T mutation. In some embodiments, a PBx transposase domain provided herein comprises a R567D mutation. In some embodiments, a PBx transposase domain provided herein comprises a M298L mutation.

In some embodiments, a PBx transposase domain comprising the M298L mutation comprises the amino acid sequence set forth in SEQ ID NO: 132.

The I182L, S301A, C420M, M185L, R315K, D421H, F200W, Q318G, N427D, V207I, E331R, Q434E, M226F, V336I, V436I, I231T, S373K, I474L, V240K, V381E, K500R, Q254N, T392S, S513P, A263E, A411N, K525P, S289A, S419T, R567D and/or M298L mutations can also be introduced into any of the truncated SPBs or PBxs (e.g., SEQ ID NOs: 28-69) provided herein. A person of skill will appreciate that the numbering of the residues depends on the size of the truncation.

The I182L, S301A, C420M, M185L, R315K, D421H, F200W, Q318G, N427D, V207I, E331R, Q434E, M226F, V336I, V436I, I231T, S373K, I474L, V240K, V381E, K500R, Q254N, T392S, S513P, A263E, A411N, K525P, S289A, S419T, R567D and/or M298L mutations can also be introduced into any of the transposase domains comprising dual CRDs provided herein. For example, an SPB transposase domain comprising the sequence set forth in SEQ ID NO:1 or 3 may further comprise a I182L, S301A, C420M, M185L, R315K, D421H, F200W, Q318G, N427D, V207I, E331R, Q434E, M226F, V336I, V436I, I231T, S373K, I474L, V240K, V381E, K500R, Q254N, T392S, S513P, A263E, A411N, K525P, S289A, S419T, R567D and/or M298L mutation (with numbering beginning at residue 12 of SEQ ID NO: 1 or 3).

Similarly, a PBx transposase domain comprising the sequence set forth in any one of SEQ ID NO: 70-72 may further comprise a I182L, S301A, C420M, M185L, R315K, D421H, F200W, Q318G, N427D, V207I, E331R, Q434E, M226F, V336I, V436I, I231T, S373K, I474L, V240K, V381E, K500R, Q254N, T392S, S513P, A263E, A411N, K525P, S289A, S419T, R567D and/or M298L mutation. In some embodiments, a PBx domain comprising dual CRDs and an M298L mutation comprises the amino acid sequence set forth in SEQ ID NO: 137

Similarly, the I182L, S301A, C420M, M185L, R315K, D421H, F200W, Q318G, N427D, V207I, E331R, Q434E, M226F, V336I, V436I, I231T, S373K, I474L, V240K, V381E, K500R, Q254N, T392S, S513P, A263E, A411N, K525P, S289A, S419T, R567D and/or M298L mutations may be introduced into any of the fusion proteins described below. For example, a fusion protein comprising a transposase domain and a DNA binding domain may further comprise a I182L, S301A, C420M, M185L, R315K, D421H, F200W, Q318G, N427D, V207I, E331R, Q434E, M226F, V336I, V436I, I231T, S373K, I474L, V240K, V381E, K500R, Q254N, T392S, S513P, A263E, A411N, K525P, S289A, S419T, R567D and/or M298L mutation. The sequences of fusion proteins comprising a LINE-targeting TAL array and an SPB transposase domain comprising a I182L, M185L, F200W, V207I, M226F, I231T, V240K, Q254N, A263E, S289A, M298L, S301A, R315K, Q318G, E331R, V336I, S373K, V381E, T392S, A411N, S419T, C420M, D421H, N427D, Q434E, V436I, I474L, K500R, S513P, K525P, R567D or mutation are set forth in SEQ ID NO: 99, 100, 101, 102, 103, 104, 105, 106, 107, 108, 109, 110, 111, 112, 113, 114, 115, 116, 117, 118, 119, 120, 121, 122, 123, 124, 125, 126, 127, 128 or 129, respectively.

### Combinations of Hyperactivity and Thermostability Mutations

The hyperactivity mutations and thermostability mutations described herein may be freely combined. Thus, a transposase domain may comprise one, two or all of the hyperactivity mutations S103P, R372H S509G and/or N571S and any or all of the thermostability mutations I182L, S301A, C420M, M185L, R315K, D421H, F200W, Q318G, N427D, V207I, E331R, Q434E, M226F, V336I, V436I, I231T, S373K, I474L, V240K, V381E, K500R, Q254N, T392S, S513P, A263E, A411N, K525P, S289A, S419T, R567D and/or M298L.

In some embodiments, an SPB transposase domain provided herein comprises an M298L thermostability mutation and one or more of the S103P, R372H S509G and/or N571S hyperactivity mutations. In some embodiments, an SPB transposase domain provided herein comprises an M298L thermostability mutation and the S103P, S509G and N571S hyperactivity mutations. In some embodiments, an SPB transposase domain provided herein comprises an M298L thermostability mutation and the S103P, R372H, S509G and N571S hyperactivity mutations. In some embodiments, a PBx transposase domain provided herein comprises an M298L thermostability mutation and one or more of the S103P, R372H S509G and/or N571S hyperactivity mutations. In some embodiments, a PBx transposase domain provided herein comprises an M298L thermostability mutation and the S103P, S509G and N571S hyperactivity mutations. In some embodiments, a PBx transposase domain provided herein comprises an M298L thermostability mutation and the S103P, R372H, S509G and N571S hyperactivity mutations.

In some embodiments, a PBx transposase domain comprising the S103P, S509G, N571S, R372H, and M298L mutations comprises the amino acid sequence set forth in SEQ ID NO: 136.

Any combination of hyperactivity and thermostability mutations can also be introduced into any of the transposase domains comprising dual CRDs provided herein. For example, an SPB transpose domain comprising the sequence set forth in SEQ ID NO: 1 or 3 may further comprise one or more of the S103P, R372H, S509G and N571S hyperactivity mutations and one or more of the I182L, S301A, C420M, M185L, R315K, D421H, F200W, Q318G, N427D, V207I, E331R, Q434E, M226F, V336I, V436I, I231T, S373K, I474L, V240K, V381E, K500R, Q254N, T392S, S513P, A263E, A411N, K525P, S289A, S419T, R567D and/or M298L thermostability mutation (with numbering beginning at residue 12 of SEQ ID NO: 1 or 3).

Similarly, a PBx transposase domain comprising the sequence set forth in any one of SEQ ID NO: 70-72 may further comprise one or more of the S103P, R372H, S509G and N571S hyperactivity mutations and one or more of the I182L, S301A, C420M, M185L, R315K, D421H, F200W, Q318G, N427D, V207I, E331R, Q434E, M226F, V336I, V436I, I231T, S373K, I474L, V240K, V381E, K500R, Q254N, T392S, S513P, A263E, A411N, K525P, S289A, S419T, R567D and/or M298L thermostability mutation. In some embodiments, a PBx domain comprising dual CRDs and a S103P, S509G, N571S, R372H, and M298L mutation comprises the amino acid sequence set forth in SEQ ID NO: 141.

Similarly, any combination of hyperactivity and thermostability mutations can be introduced into may be introduced into any of the fusion proteins described below. For example, a fusion protein comprising a transposase domain and a DNA binding domain may further comprise one or more of the S103P, R372H, S509G and N571S hyperactivity mutations and one or more of the I182L, S301A, C420M, M185L, R315K, D421H, F200W, Q318G, N427D, V207I, E331R, Q434E, M226F, V336I, V436I, I231T, S373K, I474L, V240K, V381E, K500R, Q254N, T392S, S513P, A263E, A411N, K525P, S289A, S419T, R567D and/or M298L thermostability mutation.

### Fusion Proteins Comprising Transposase Domains

Also provided herein are fusion proteins comprising one or more transposase domains described herein.

In some embodiments, provided herein is a fusion protein comprising an SPB or PBx domain comprising a second C-terminal CRD domain, and a DNA targeting domain. DNA targeting domains are described further below. In some embodiments, provided herein is a fusion protein comprising an N-terminally deleted SPB or PBx domain comprising a second C-terminal CRD domain, and a DNA targeting domain and a protein stabilization domain (PSD). PSDs are described further below.

In some embodiments, a fusion protein provided herein comprises, in N-terminal to C-terminal order, a PSD, a DNA targeting domain, and a transposase domain comprising an N-terminal deletion and dual CRD domains.

### DNA Targeting Domains

The transposase domains and fusion proteins provided herein may further comprise one or more DNA targeting domains. A DNA-targeting domain may be attached to the C-terminus or the N-terminus of the transposase domain of the fusion protein. In preferred embodiments, the DNA-targeting domain is attached to the N-terminus of the transposase domain, e.g., a transposase domain comprising an N-terminal deletion. Without wishing to be bound by theory, it is believed that addition a DNA targeting domain to a transposase domain improves site-specific transposase activity by targeting the transposase fused to the DNA targeting domain to the targeted site. In some embodiments, the insertion of a DNA targeting domain improves site-specific transposase activity by at least 2-fold, at least 3-fold, at least 4-fold, or at least 5-fold compared to the same transposase domain not comprising a DNA targeting domain.

Any DNA targeting domain known in the art may be used in the context of the transposase domains, fusion proteins, and tandem dimer transposases described herein, including, without limitation, CRISPR, Zinc Finger Motifs, TALE, and transcription factors. In some embodiments, the DNA targeting domain comprises one, two, or three Zinc Finger Motifs. In some embodiments, the three Zinc Finger Motifs are flanked by GGGGS linkers. In some embodiments, the three Zinc Finger Motifs flanked by GGGGS linkers cumulatively comprise the sequence set forth in SEQ ID NO: 74: GGGGSERPYACPVESCDRRFSRSDELTRHIRIHTGQKPFQCRICMRNFSRSDHLTTHIRTH TGEKPFACDICGRKFARSDERKRHTKIHLRQKDGGGGS (SEQ ID NO: 74) or a sequence having at least 75%, at least 80%, at least 85%, at least 90%, at least 95%, at least 98%, or at least 99% identity thereto.

In a specific embodiment, provided herein is a fusion protein comprising a transposase domain comprises an N-terminal deletion, an NLS, and three Zinc Finger Motifs. In some embodiments, the NLS comprises or consists of the sequence set forth in SEQ ID NO: 88.

In some aspects, the DNA targeting domain is a TAL array. TALEs (Transcription activator-like effectors) from Xanthomonas typically contain a 288 amino acid N-terminus followed by an array of a variable number of ˜34 amino acid repeats followed by a 278 amino acid C-terminus; however, truncated versions have been described in the literature (e.g., see Miller et al., Nat Biotechnol 29, 143-148 (2011). TALs fused to a FokI nuclease (called TALENs) most often contain truncations of the N and C terminus. For example, the first 152 amino acids of the N-terminus is often removed leaving 136 amino acids remaining (called Delta 152; SEQ ID No 75) and the C-terminus is often truncated leaving 63 amino acids (called +63; SEQ ID NO: 76).

TALs contain arrays of 34 amino acids repeated a variable number of times. Two amino acids at position 12 and 13 are varied and determine which nucleotide the TAL repeat will recognize. This feature allows a TAL array to be programed to bind a specific DNA sequence. The amino acids NG recognize T, NI recognize A, NN recognize G or A, HD recognize C, NK recognize G, NS recognize A, C, G or T. When the two amino acids at position 12 and 13 are replaced with NS, NA, or with a single S, the TAL module recognizes any of the four nucleotides indiscriminately. When the two amino acids at position 12 and 13 are replaced with a single N, the module can accommodate recognition of a 5-Methylcytosine (5mC), such as those that occur in mammalian genomes at CpG sequences. Other amino acids within the 34 residue repeat may also be varied. For example position 11 is often changed to an N for repeats that recognize G. Also, positions 4 and 32 are often varied to reduce the repetitiveness of the array but not to determine the binding specificity. The number of 34 amino acid repeats in an array determines the length of the DNA sequence recognized (one protein repeat binds one DNA bp). Furthermore, the last bp is recognized by a “half array” that is 20 amino acids rather than 34.

In addition, the N-terminal domain of TALs (e.g., SEQ ID NO: 75) recognizes and requires a T that is located immediately 5′ of the target DNA sequence. Mutations of TAL N-terminal domains have been described in the literature that no longer require a 5′ T (Lamb et al., Nucleic Acids Res. 2013 November; 41(21):9779-85. doi: 10.1093/nar/gkt754. Epub 2013 Aug. 26. PMID: 23980031; PMCID: PMC3834825.) For example, the NT-G mutant requires a 5′G instead of a 5′T (SEQ ID NO: 77) while the NT-βN mutant does not require any specific 5′ nucleotide (SEQ ID NO: 78). These mutated N-terminal domain sequences may be used to provide additional sequence options that may be targeted using TAL Arrays.

Exemplary TAL modules are set forth in SEQ ID NOs: 79-82, wherein X is any amino acid:

TAL Module Version 1:
(SEQ ID NO: 79)
LTPDQVVAIAXXXGGKQALETVQRLLPVLCQDHG
TAL Module Version 2:
(SEQ ID NO: 80)
LTPEQVVAIAXXXGGKQALETVQRLLPVLCQAHG
TAL Module Version 3″
(SEQ ID NO: 81)
LTPDQVVAIAXXXGGKQALETVQRLLPVLCQAHG
TAL Module Version 4:
(SEQ ID NO: 82)
LTPAQVVAIAXXXGGKQALETVQRLLPVLCQDHG.

An exemplary TAL Half Module is set forth in SEQ ID NO: 83, wherein X is any amino acid: LTPEQVVAIAXXXGGRPALE.

Pairs of TAL arrays targeting sequences in the desired gene may be designed and the corresponding modules selected and pooled together using “Golden Gate Assembly,” to assemble in frame each TAL-Array. Alternatively, nucleotide sequences encoding assembled TAL arrays may be synthesized de novo. The DNA sequence encoding TAL Arrays generated herein may be further codon optimized using GeneArt algorithms (Thermo Fisher).

When designing left and right TAL Arrays comprising a N-terminal domain recognizing a T and a TAL C-terminal domain to be fused to an N-terminal deleted transposase sequence (i.e., TAL-ssSPB or TAL-PBx; described below), one TAL Array recognizes a sequence 5′ of the TTAA and the other TAL Array recognizes a sequence 3′ of the TTAA. Since the sequence 5′ of TTAA is most often different from the sequence 3′ of TTAA in genomic DNA targets, TAL-ssSPB will most often be used as a heterodimer consisting of two different TAL domains that recognize two different DNA sequences. Additionally, the sequence recognized by the TAL Array is not directly adjacent to the TTAA. Instead, it is separated from the TTAA by a spacer of a given bp length, e.g., spacers of 12 bp, 13 bp or 14 bp.

A TAL array may target any DNA sequence (e.g., genomic DNA sequence) of interest. It will be apparent to a person of skill in the art that any left TAL array for a given target can be combined with any right TAL array for the same target.

In some embodiments, a TAL array targets ZFN268. An illustrative sequence of a TAL array targeting ZFN268, which serves as the left and the right array, is set forth in SEQ ID NO: 84. In some embodiments, the TAL array targeting ZFN268 binds to a nucleic acid molecule comprising the sequence set forth in SEQ ID NO: 85.

In some embodiments, a TAL array targets a LINE1 repeat element. An illustrative sequence of left TAL arrays targeting a LINE1 repeat element is set forth in SEQ ID NO: 89:

(SEQ ID NO: 89)
LTPDQVVAIASHDGGKQALETVQRLLPVLCQDHGLTPEQVVAIASHDGGKQA
LETVQRLLPVLCQAHGLTPDQVVAIASNGGGKQALETVQRLLPVLCQAHGLTPAQVVAI
ASNIGGKQALETVQRLLPVLCQDHGLTPDQVVAIANNNGGKQALETVQRLLPVLCQDH
GLTPEQVVAIASNGGGKQALETVQRLLPVLCQAHGLTPDQVVAIASHDGGKQALETVQ
RLLPVLCQAHGLTPAQVVAIASNGGGKQALETVQRLLPVLCQDHGLTPDQVVAIASHD
GGKQALETVQRLLPVLCQDHGLTPEQVVAIASNGGGRPALE

An illustrative sequence of right TAL arrays targeting LINE1 is set forth in SEQ ID NO: 90:

(SEQ ID NO: 90)
LTPDQVVAIASNGGGKQALETVQRLLPVLCQDHGLTPEQVVAIANNNGGKQALETVQR
LLPVLCQAHGLTPDQVVAIASNGGGKQALETVQRLLPVLCQAHGLTPAQVVAIASHDG
GKQALETVQRLLPVLCQDHGLTPDQVVAIASNGGGKQALETVQRLLPVLCQDHGLTPE
QVVAIASHDGGKQALETVQRLLPVLCQAHGLTPDQVVAIASNGGGKQALETVQRLLPV
LCQAHGLTPAQVVAIASNGGGKQALETVQRLLPVLCQDHGLTPDQVVAIASNGGGKQA
LETVQRLLPVLCQDHGLTPEQVVAIASNGGGRPALE

The DNA targeting domain may be fused or linked to the N-terminus of a transposase domain comprising an N-terminal deletion. For example, the DNA targeting domain may be inserted into a transposase domain at a suitable position in the N-terminal region of the transposase domain.

The DNA targeting domain may be inserted into the N-terminus of a transposase domain. In some embodiments, the DNA targeting domain is inserted between the 82^{nd} and 83^{rd} amino acid of one of SEQ ID NOs: 71-73. In some embodiments, the DNA targeting domain is inserted between the 83^{rd} and 84^{th} amino acid of one of SEQ ID NOs: 71-73. In some embodiments, the DNA targeting domain is inserted between the 84^{th} and 85^{th} amino acid of one of SEQ ID NOs: 71-73. In some embodiments, the DNA targeting domain is inserted between the 85^{th} and 86^{th} amino acid of one of SEQ ID NOs: 71-73. In some embodiments, the DNA targeting domain is inserted between the 86^{th} and 87^{th} amino acid of one of SEQ ID NOs: 71-73. In some embodiments, the DNA targeting domain is inserted between the 87^{th} and 88^{th} amino acid of one of SEQ ID NOs: 71-73. In some embodiments, the DNA targeting domain is inserted between the 88^{th} and 89^{th} amino acid of one of SEQ ID NOs: 71-73. In some embodiments, the DNA targeting domain is inserted between the 89^{th} and 90^{th} amino acid of one of SEQ ID NOs: 71-73. In some embodiments, the DNA targeting domain is inserted between the 90^{th} and 91^{st} amino acid of one of SEQ ID NOs: 71-73. In some embodiments, the DNA targeting domain is inserted between the 91^{st} and 92^{nd} amino acid of one of SEQ ID NOs: 71-73. In some embodiments, the DNA targeting domain is inserted between the 92^{nd} and 93^{rd} amino acid of one of SEQ ID NOs: 71-73. In some embodiments, the DNA targeting domain is inserted between the 93^{rd} and 94^{th} amino acid of one of SEQ ID NOs: 71-73. In some embodiments, the DNA targeting domain is inserted between the 94^{th} and 95^{th} amino acid of one of SEQ ID NOs: 71-73.

In some embodiments, the DNA targeting domain is inserted between the 95^{th} and 96^{th} amino acid of one of SEQ ID NOs: 71-73. In some embodiments, the DNA targeting domain is inserted between the 96^{th} and 97^{th} amino acid of one of SEQ ID NOs: 71-73. In some embodiments, the DNA targeting domain is inserted between the 97^{th} and 98^{th} amino acid of one of SEQ ID NOs: 71-73. In some embodiments, the DNA targeting domain is inserted between the 98^{th} and 99^{th} amino acid of one of SEQ ID NOs: 71-73. In some embodiments, the DNA targeting domain is inserted between the 99^{th} and 100^{th} amino acid of one of SEQ ID NOs: 71-73. In some embodiments, the DNA targeting domain is inserted between the 100^{th} and 101^{st} amino acid of one of SEQ ID NOs: 71-73. In some embodiments, the DNA targeting domain is inserted between the 101^{st} and 102^{nd} amino acid of one of SEQ ID NOs: 71-73. In some embodiments, the DNA targeting domain is inserted between the 102^{nd} and 103^{rd} amino acid of one of SEQ ID NOs: 71-73. In some embodiments, the DNA targeting domain is inserted between the 103^{rd} and 104^{th} amino acid of one of SEQ ID NOs: 71-73. In some embodiments, the DNA targeting domain is inserted between the 104 and 105^{th} amino acid of one of SEQ ID NOs: 71-73. In some embodiments, the DNA targeting domain comprises the sequence of SEQ ID NO: 74 or a sequence having at least 75%, at least 80%, at least 85%, at least 90%, at least 95%, at least 98%, or at least 99% identity thereto. In some embodiments, the DNA targeting domain comprises a TAL. The transposase domain may further comprise an NLS.

In some embodiments, the DNA targeting domain replaces the 83^{rd} amino acid of one of SEQ ID NOs: 71-73. In some embodiments, the DNA targeting domain replaces the 84^{th} amino acid of one of SEQ ID NOs: 71-73. In some embodiments, the DNA targeting domain replaces the 85^{th} amino acid of one of SEQ ID NOs: 71-73. In some embodiments, the DNA targeting domain replaces the 86^{th} amino acid of one of SEQ ID NOs: 71-73. In some embodiments, the DNA targeting domain replaces the 87^{th} amino acid of one of SEQ ID NOs: 71-73. In some embodiments, the DNA targeting domain replaces the 88^{th} amino acid of one of SEQ ID NOs: 71-73. In some embodiments, the DNA targeting domain replaces the 89^{th} amino acid of one of SEQ ID NOs: 71-73. In some embodiments, the DNA targeting domain replaces the 90^{th} amino acid of one of SEQ ID NOs: 71-73. In some embodiments, the DNA targeting domain replaces the 91^{st} amino acid of one of SEQ ID NOs: 71-73. In some embodiments, the DNA targeting domain replaces the 92^{nd} amino acid of one of SEQ ID NOs: 71-73. In some embodiments, the DNA targeting domain replaces the 93^{rd} amino acid of one of SEQ ID NOs: 71-73. In some embodiments, the DNA targeting domain replaces the 94^{th} amino acid of one of SEQ ID NOs: 71-73. In some embodiments, the DNA targeting domain replaces the 95^{th} amino acid of one of SEQ ID NOs: 71-73. In some embodiments, the DNA targeting domain replaces the 96^{th} amino acid of one of SEQ ID NOs: 71-73. In some embodiments, the DNA targeting domain replaces the 97^{th} amino acid of one of SEQ ID NOs: 71-73. In some embodiments, the DNA targeting domain replaces the 98^{th} amino acid of one of SEQ ID NOs: 71-73. In some embodiments, the DNA targeting domain replaces the 99^{th} amino acid of one of SEQ ID NOs: 71-73. In some embodiments, the DNA targeting domain replaces the 100^{th} amino acid of one of SEQ ID NOs: 71-73. In some embodiments, the DNA targeting domain replaces the 101^{st} amino acid of one of SEQ ID NOs: 71-73. In some embodiments, the DNA targeting domain replaces the 102^{nd} amino acid of one of SEQ ID NOs: 71-73. In some embodiments, the DNA targeting domain replaces the 103^{rd} amino acid of one of SEQ ID NOs: 71-73. In some embodiments, the DNA targeting domain replaces the 104^{th} amino acid of one of SEQ ID NOs: 71-73. In some embodiments, the DNA targeting domain replaces the 105^{th} amino acid of one of SEQ ID NOs: 71-73. In some embodiments, the DNA targeting domain comprises the sequence of SEQ ID NO: 74 or a sequence having at least 75%, at least 80%, at least 85%, at least 90%, at least 95%, at least 98%, or at least 99% identity thereto. In some embodiments, the DNA targeting domain comprises a TAL. The transposase domain may further comprise an NLS.

An exemplary sequence of a fusion protein comprising a transposase domain comprising dual CRD domains and an N-terminal deletion of 93 amino acids, an NLS, and three Zinc Finger Motifs flanked by GGGGS linkers is show in SEQ ID NO: 86, where the NLS is shown in italics, the sequence comprising the three Zinc Finger Motifs and GGGGS linkers is underlined, and the transposase domain comprising an N-terminal deletion of 93 amino acid is shown in bold, the CRD linker sequence in lower case font and the second CRD domain (SEQ ID NO: 2) in bold italics:

(SEQ ID NO: 86)
MAPKKKRKVGGGGSERPYACPVESCDRRFSRSDELTRHIRIHTGQKPFQCRICMRNFSRS
DHLTTHIRTHTGEKPFACDICGRKFARSDERKRHTKIHLRQKDGGGGSNKHCWSTSKS
TRRSRVSALNIVRSQRGPTRMCRNIYDPLLCFKLFFTDENISEIVKWTNAEISLKRRE
SMTSATFRDTNEDEIYAFFGILVMTAVRKDNHMSTDDLFDRSLSMVYVSVMSRDRF
DFLIRCLRMDDKSIRPTLRENDVFTPVRKIWDLFIHQCIQNYTPGAHLTIDEQLLGF
RGRCPFRVYIPNKPSKYGIKILMMCDSGTKYMINGMPYLGRGTQTNGVPLGEYYV
KELSKPVHGSCRNITCDNWFTSIPLAKNLLQEPYKLTIVGTVRSNKREIPEVLKNSR
SRPVGTSMFCFDGPLTLVSYKPKPAKMVYLLSSCDEDASINESTGKPQMVMYYNQ
TKGGVDTLDQMCSVMTCSRKTNRWPMALLYGMINIACINSFIIYSHNVSSKGEKVQ
SRKKFMRNLYMSLTSSFMRKRLEAPTLKRYLRDNISNILPKEVPGTSDDSTEEPVM
KKRTYCTYCPSKIRRKANASCKKCKKVICREHNIDMCQSCFagggSTEEPVMKKRTY
CTYCPSKIRRKANASCKKCKKVICREHNIDMCQSCF

An exemplary sequence of a fusion protein comprising an integration deficient transposase domain comprising dual CRD domains and an N-terminal deletion of 93 amino acids, an NLS, and three Zinc Finger Motifs flanked by GGGGS linkers is set forth in SEQ ID NO: 87, where the NLS is shown in italics, the sequence comprising the three Zinc Finger Motifs and GGGGS linkers is underlined, and the transposase domain comprising an N-terminal deletion of 93 amino acid is shown in bold, the linker sequence shown in lower case font and the second CRD domain shown in bold italics:

(SEQ ID NO: 87)
MAPKKKRKVGGGGSERPYACPVESCDRRFSRSDELTRHIRIHTGQKPFQCRICMRNFSRS
DHLTTHIRTHTGEKPFACDICGRKFARSDERKRHTKIHLRQKDGGGGSNKHCWSTSKS
TRRSRVSALNIVRSQRGPTRMCRNIYDPLLCFKLFFTDEIISEIVKWTNAEISLKRRE
SMTSATFRDTNEDEIYAFFGILVMTAVRKDNHMSTDDLFDRSLSMVYVSVMSRDRF
DFLIRCLRMDDKSIRPTLRENDVFTPVRKIWDLFIHQCIQNYTPGAHLTIDEQLLGF
RGRCPFRVYIPNKPSKYGIKILMMCDSGTKYMINGMPYLGRGTQTNGVPLGEYYV
KELSKPVHGSCRNITCDNWFTSIPLAKNLLQEPYKLTIVGTVASNAREIPEVLKNSR
SRPVGTSMFCFDGPLTLVSYKPKPAKMVYLLSSCDEDASINESTGKPQMVMYYNQ
TKGGVDTLNQMCSVMTCSRKTNRWPMALLYGMINIACINSFIIYSHNVSSKGEKVQ
SRKKFMRNLYMSLTSSFMRKRLEAPTLKRYLRDNISNILPKEVPGTSDDSTEEPVM
KKRTYCTYCPSKIRRKANASCKKCKKVICREHNIDMCQSCFagggSTEEPVMKKRTY
CTYCPSKIRRKANASCKKCKKVICREHNIDMCQSCF.

Exemplary sequences of fusion proteins comprising an integration deficient transposase domain comprising dual CRD domains and an N-terminal deletion of 93 amino acids, an NLS and a LINE1 left L2 TAL Array are set forth in SEQ ID NOs: 17 and 19:

(SEQ ID NO: 17)
MDYKDHDGDYKDHDIDYKDDDDKMAPKKKRKVGIHRGVPMVDLRTLGYSQQQQEKI
KPKVRSTVAQHHEALVGHGFTHAHIVALSQHPAALGTVAVKYQDMIAALPEATHEAIV
GVGKQWSGARALEALLTVAGELRGPPLQLDTGQLLKIAKRGGVTAVEAVHAWRNALT
GAPLNLTPDQVVAIASHDGGKQALETVQRLLPVLCQDHGLTPEQVVAIASHDGGKQAL
ETVQRLLPVLCQAHGLTPDQVVAIASNGGGKQALETVQRLLPVLCQAHGLTPAQVVAI
ASNIGGKQALETVQRLLPVLCQDHGLTPDQVVAIANNNGGKQALETVQRLLPVLCQDH
GLTPEQVVAIASNGGGKQALETVQRLLPVLCQAHGLTPDQVVAIASHDGGKQALETVQ
RLLPVLCQAHGLTPAQVVAIASNGGGKQALETVQRLLPVLCQDHGLTPDQVVAIASHD
GGKQALETVQRLLPVLCQDHGLTPEQVVAIASNGGGRPALESIVAQLSRPDPALAALTN
DHLVALACLGGRPALDAVKKGLPHAPALIKRTNRRIPERTSHRVADHAQVVRVLGGGG
GSPQRTIRGKNKHCWSTSKSTRRSRVSALNIVRSQRGPTRMCRNIYDPLLCFKLFFTDEII
SEIVKWTNAEISLKRRESMTSATFRDTNEDEIYAFFGILVMTAVRKDNHMSTDDLFDRSL
SMVYVSVMSRDRFDFLIRCLRMDDKSIRPTLRENDVFTPVRKIWDLFIHQCIQNYTPGAH
LTIDEQLLGFRGRCPFRVYIPNKPSKYGIKILMMCDSGTKYMINGMPYLGRGTQTNGVP
LGEYYVKELSKPVHGSCRNITCDNWFTSIPLAKNLLQEPYKLTIVGTVASNAREIPEVLK
NSRSRPVGTSMFCFDGPLTLVSYKPKPAKMVYLLSSCDEDASINESTGKPQMVMYYNQ
TKGGVDTLNQMCSVMTCSRKTNRWPMALLYGMINIACINSFIIYSHNVSSKGEKVQSRK
KFMRNLYMSLTSSFMRKRLEAPTLKRYLRDNISNILPKEVPGTSDDSTEEPVMKKRTYC
TYCPSKIRRKANASCKKCKKVICREHNIDMCQSCFAGGGSTEEPVMKKRTYCTYCPSKI
RRKANASCKKCKKVICREHNIDMCQSCF
(SEQ ID NO: 19)
MDYKDHDGDYKDHDIDYKDDDDKMAPKKKRKVGIHRGVPMVDLRTLGYSQQQQEKI
KPKVRSTVAQHHEALVGHGFTHAHIVALSQHPAALGTVAVKYQDMIAALPEATHEAIV
GVGKQWSGARALEALLTVAGELRGPPLQLDTGQLLKIAKRGGVTAVEAVHAWRNALT
GAPLNLTPDQVVAIASHDGGKQALETVQRLLPVLCQDHGLTPEQVVAIASHDGGKQAL
ETVQRLLPVLCQAHGLTPDQVVAIASNGGGKQALETVQRLLPVLCQAHGLTPAQVVAI
ASNIGGKQALETVQRLLPVLCQDHGLTPDQVVAIANNNGGKQALETVQRLLPVLCQDH
GLTPEQVVAIASNGGGKQALETVQRLLPVLCQAHGLTPDQVVAIASHDGGKQALETVQ
RLLPVLCQAHGLTPAQVVAIASNGGGKQALETVQRLLPVLCQDHGLTPDQVVAIASHD
GGKQALETVQRLLPVLCQDHGLTPEQVVAIASNGGGRPALESIVAQLSRPDPALAALTN
DHLVALACLGGRPALDAVKKGLPHAPALIKRTNRRIPERTSHRVADHAQVVRVLGGGG
GSPQRTIRGKNKHCWSTSKSTRRSRVSALNIVRSQRGPTRMCRNIYDPLLCFKLFFTDEII
SEIVKWTNAEISLKRRESMTSATFRDTNEDEIYAFFGILVMTAVRKDNHMSTDDLFDRSL
SMVYVSVMSRDRFDFLIRCLRMDDKSIRPTLRENDVFTPVRKIWDLFIHQCIQNYTPGAH
LTIDEQLLGFRGRCPFRVYIPNKPSKYGIKILMMCDSGTKYMINGMPYLGRGTQTNGVP
LGEYYVKELSKPVHGSCRNITCDNWFTSIPLAKNLLQEPYKLTIVGTVASNAREIPEVLK
NSRSRPVGTSMFCFDGPLTLVSYKPKPAKMVYLLSSCDEDASINESTGKPQMVMYYNQ
TKGGVDTLNQMCSVMTCSRKTNRWPMALLYGMINIACINSFIIYSHNVSSKGEKVQSRK
KFMRNLYMSLTSSFMRKRLEAPTLKRYLRDNISNILPKEVPGTSDDSTEEPVMKKRTYC
TYCPSKIRRKANASCKKCKKVICREHNIDMCQSCFGTSDDSTEEPVMKKRTYCTYCPSKI
RRKANASCKKCKKVICREHNIDMCQSCF

Illustrative sequences of fusion proteins comprising an integration deficient transposase domain comprising dual CRD domains and an N-terminal deletion of 93 amino acids, a NLS and a LINE1 right TAL Array are set forth in SEQ ID NOs: 18 and 20:

(SEQ ID NO: 18)
MDYKDHDGDYKDHDIDYKDDDDKMAPKKKRKVGIHRGVPMVDLRTLGYSQQQQEKI
KPKVRSTVAQHHEALVGHGFTHAHIVALSQHPAALGTVAVKYQDMIAALPEATHEAIV
GVGKQWSGARALEALLTVAGELRGPPLQLDTGQLLKIAKRGGVTAVEAVHAWRNALT
GAPLNLTPDQVVAIASNGGGKQALETVQRLLPVLCQDHGLTPEQVVAIANNNGGKQAL
ETVQRLLPVLCQAHGLTPDQVVAIASNGGGKQALETVQRLLPVLCQAHGLTPAQVVAI
ASHDGGKQALETVQRLLPVLCQDHGLTPDQVVAIASNGGGKQALETVQRLLPVLCQDH
GLTPEQVVAIASHDGGKQALETVQRLLPVLCQAHGLTPDQVVAIASNGGGKQALETVQ
RLLPVLCQAHGLTPAQVVAIASNGGGKQALETVQRLLPVLCQDHGLTPDQVVAIASNG
GGKQALETVQRLLPVLCQDHGLTPEQVVAIASNGGGRPALESIVAQLSRPDPALAALTN
DHLVALACLGGRPALDAVKKGLPHAPALIKRTNRRIPERTSHRVADHAQVVRVLGGGG
GSPQRTIRGKNKHCWSTSKSTRRSRVSALNIVRSQRGPTRMCRNIYDPLLCFKLFFTDEII
SEIVKWTNAEISLKRRESMTSATFRDTNEDEIYAFFGILVMTAVRKDNHMSTDDLFDRSL
SMVYVSVMSRDRFDFLIRCLRMDDKSIRPTLRENDVFTPVRKIWDLFIHQCIQNYTPGAH
LTIDEQLLGFRGRCPFRVYIPNKPSKYGIKILMMCDSGTKYMINGMPYLGRGTQTNGVP
LGEYYVKELSKPVHGSCRNITCDNWFTSIPLAKNLLQEPYKLTIVGTVASNAREIPEVLK
NSRSRPVGTSMFCFDGPLTLVSYKPKPAKMVYLLSSCDEDASINESTGKPQMVMYYNQ
TKGGVDTLNQMCSVMTCSRKTNRWPMALLYGMINIACINSFIIYSHNVSSKGEKVQSRK
KFMRNLYMSLTSSFMRKRLEAPTLKRYLRDNISNILPKEVPGTSDDSTEEPVMKKRTYC
TYCPSKIRRKANASCKKCKKVICREHNIDMCQSCFAGGGSTEEPVMKKRTYCTYCPSKI
RRKANASCKKCKKVICREHNIDMCQSCF
(SEQ ID NO: 20)
MDYKDHDGDYKDHDIDYKDDDDKMAPKKKRKVGIHRGVPMVDLRTLGYSQQQQEKI
KPKVRSTVAQHHEALVGHGFTHAHIVALSQHPAALGTVAVKYQDMIAALPEATHEAIV
GVGKQWSGARALEALLTVAGELRGPPLQLDTGQLLKIAKRGGVTAVEAVHAWRNALT
GAPLNLTPDQVVAIASNGGGKQALETVQRLLPVLCQDHGLTPEQVVAIANNNGGKQAL
ETVQRLLPVLCQAHGLTPDQVVAIASNGGGKQALETVQRLLPVLCQAHGLTPAQVVAI
ASHDGGKQALETVQRLLPVLCQDHGLTPDQVVAIASNGGGKQALETVQRLLPVLCQDH
GLTPEQVVAIASHDGGKQALETVQRLLPVLCQAHGLTPDQVVAIASNGGGKQALETVQ
RLLPVLCQAHGLTPAQVVAIASNGGGKQALETVQRLLPVLCQDHGLTPDQVVAIASNG
GGKQALETVQRLLPVLCQDHGLTPEQVVAIASNGGGRPALESIVAQLSRPDPALAALTN
DHLVALACLGGRPALDAVKKGLPHAPALIKRTNRRIPERTSHRVADHAQVVRVLGGGG
GSPQRTIRGKNKHCWSTSKSTRRSRVSALNIVRSQRGPTRMCRNIYDPLLCFKLFFTDEII
SEIVKWTNAEISLKRRESMTSATFRDTNEDEIYAFFGILVMTAVRKDNHMSTDDLFDRSL
SMVYVSVMSRDRFDFLIRCLRMDDKSIRPTLRENDVFTPVRKIWDLFIHQCIQNYTPGAH
LTIDEQLLGFRGRCPFRVYIPNKPSKYGIKILMMCDSGTKYMINGMPYLGRGTQTNGVP
LGEYYVKELSKPVHGSCRNITCDNWFTSIPLAKNLLQEPYKLTIVGTVASNAREIPEVLK
NSRSRPVGTSMFCFDGPLTLVSYKPKPAKMVYLLSSCDEDASINESTGKPQMVMYYNQ
TKGGVDTLNQMCSVMTCSRKTNRWPMALLYGMINIACINSFIIYSHNVSSKGEKVQSRK
KFMRNLYMSLTSSFMRKRLEAPTLKRYLRDNISNILPKEVPGTSDDSTEEPVMKKRTYC
TYCPSKIRRKANASCKKCKKVICREHNIDMCQSCFGTSDDSTEEPVMKKRTYCTYCPSKI
RRKANASCKKCKKVICREHNIDMCQSCF

### Protein Stabilization Domains

In some embodiments, a fusion protein provided herein may further comprise a protein stabilization domain (PSD). The PSD is preferably attached to the N-terminus of the DNA targeting domain if present. Without wishing to be bound by theory, it is believed that the addition of a PSD can enhance protein stability or enhance stability of the transposase tetramer-DNA complex.

The PSD may be of approximately the same size as the N-terminal deletion in the transposase domain. For example, in some embodiments, the N-terminal deletion of transposase domain comprises amino acids 1-93, and the PSD comprises 92 amino acids.

In some embodiments, the PSD comprises amino acids 1-83 of SEQ ID NO: 73. In some embodiments, the PSD comprises amino acids 1-83 of SEQ ID NOs: 71 or 72. In some embodiments, the PSD comprises amino acids 1-84 of SEQ ID NO: 73. In some embodiments, the PSD comprises amino acids 1-84 of SEQ ID NOs: 71 or 72. In some embodiments, the PSD comprises amino acids 1-85 of SEQ ID NO: 73. In some embodiments, the PSD comprises amino acids 1-85 of SEQ ID NOs: 71 or 72. In some embodiments, the PSD comprises amino acids 1-86 of SEQ ID NO: 73. In some embodiments, the PSD comprises amino acids 1-86 of SEQ ID NOs: 71 or 72. In some embodiments, the PSD comprises amino acids 1-87 of SEQ ID NO: 73. In some embodiments, the PSD comprises amino acids 1-87 of SEQ ID NOs: 71 or 72. In some embodiments, the PSD comprises amino acids 1-88 of SEQ ID NO: 73. In some embodiments, the PSD comprises amino acids 1-88 of SEQ ID NOs: 71 or 72. In some embodiments, the PSD comprises amino acids 1-89 of SEQ ID NO: 73. In some embodiments, the PSD comprises amino acids 1-89 of SEQ ID NOs: 71 or 72. In some embodiments, the PSD comprises amino acids 1-90 of SEQ ID NO: 73. In some embodiments, the PSD comprises amino acids 1-90 of SEQ ID NOs: 71 or 72. In some embodiments, the PSD comprises amino acids 1-91 of SEQ ID NO: 73. In some embodiments, the PSD comprises amino acids 1-91 of SEQ ID NOs: 71 or 72. In some embodiments, the PSD comprises amino acids 1-92 of SEQ ID NO: 73. In some embodiments, the PSD comprises amino acids 1-92 of SEQ ID NOs: 71 or 72. In some embodiments, the PSD comprises amino acids 1-93 of SEQ ID NO: 73. In some embodiments, the PSD comprises amino acids 1-93 of SEQ ID NOs: 71 or 72. In some embodiments, the PSD comprises amino acids 1-94 of SEQ ID NO: 73. In some embodiments, the PSD comprises amino acids 1-94 of SEQ ID NOs: 71 or 72. In some embodiments, the PSD comprises amino acids 1-95 of SEQ ID NO: 73. In some embodiments, the PSD comprises amino acids 1-95 of SEQ ID NOs: 71 or 72. In some embodiments, the PSD comprises amino acids 1-96 of SEQ ID NO: 73. In some embodiments, the PSD comprises amino acids 1-96 of SEQ ID NOs: 71 or 72. In some embodiments, the PSD comprises amino acids 1-97 of SEQ ID NO: 73. In some embodiments, the PSD comprises amino acids 1-97 of SEQ ID NOs: 71 or 72. In some embodiments, the PSD comprises amino acids 1-98 of SEQ ID NO: 73. In some embodiments, the PSD comprises amino acids 1-98 of SEQ ID NOs: 71 or 72. In some embodiments, the PSD comprises amino acids 1-99 of SEQ ID NO: 73. In some embodiments, the PSD comprises amino acids 1-99 of SEQ ID NOs: 71 or 72. In some embodiments, the PSD comprises amino acids 1-100 of SEQ ID NO: 73. In some embodiments, the PSD comprises amino acids 1-100 of SEQ ID NOs: 71 or 72. In some embodiments, the PSD comprises amino acids 1-101 of SEQ ID NO: 73. In some embodiments, the PSD comprises amino acids 1-101 of SEQ ID NOs: 71 or 72. In some embodiments, the PSD comprises amino acids 1-102 of SEQ ID NO: 73. In some embodiments, the PSD comprises amino acids 1-102 of SEQ ID NOs: 71 or 72. In some embodiments, the PSD comprises amino acids 1-103 of SEQ ID NO: 73. In some embodiments, the PSD comprises amino acids 1-103 of SEQ ID NOs: 71 or 72.

In some embodiments, the PSD comprises the sequence

(SEQ ID NO: 91)
GGSSLDDEHILSALLQSDDELVGEDSDSEVSDHVSEDDVQSDTEEAFID
EVHEVQPTSSGSEILDEQNVIEQPGSSLASNRILTLPQRTIRG.

Thus, provided herein are fusion proteins comprising, in N-terminal to C-terminal order: a nuclear localization signal (NLS), PSD, a DNA targeting domain, and a transposase domain comprising an N-terminal deletion as compared to the sequence set forth in SEQ ID NOs: 71-73.

Exemplary sequences of fusion proteins comprising a PSD, an NLS, a DNA targeting domain and a transposase domain comprising an N-terminal deletion and a second C-terminal CRD domain are shown in SEQ ID NOs: 92 (PBx transposase domain) and 93 (SPB transposase domain) with the NLS (here: PKKKRKV) shown in italics, the PSD shown in bold and underlined, the DNA targeting domain (here: three Zinc Finger Motifs flanked by GGGGS linkers) underlined, the N-terminally deleted transposase domain (here: PBx) shown in bold, the linker sequence in lower case font and the second CRD domain in bold italics:

(SEQ ID NO: 92)
MAPKKKRKVGGGGSSLDDEHILSALLQSDDELVGEDSDSEVSDHVSEDDVQSDTEEA
FIDEVHEVQPTSSGSEILDEQNVIEQPGSSLASNRILTLPQRTIRGGGGGSERPYACPVE
SCDRRFSRSDELTRHIRIHTGQKPFQCRICMRNFSRSDHLTTHIRTHTGEKPFACDICGRKF
ARSDERKRHTKIHLRQKDGGGGSNKHCWSTSKSTRRSRVSALNIVRSQRGPTRMCRN
IYDPLLCFKLFFTDEIISEIVKWTNAEISLKRRESMTSATFRDTNEDEIYAFFGILVMT
AVRKDNHMSTDDLFDRSLSMVYVSVMSRDRFDFLIRCLRMDDKSIRPTLRENDVFT
PVRKIWDLFIHQCIQNYTPGAHLTIDEQLLGFRGRCPFRVYIPNKPSKYGIKILMMC
DSGTKYMINGMPYLGRGTQTNGVPLGEYYVKELSKPVHGSCRNITCDNWFTSIPL
AKNLLQEPYKLTIVGTVASNAREIPEVLKNSRSRPVGTSMFCFDGPLTLVSYKPKPA
KMVYLLSSCDEDASINESTGKPQMVMYYNQTKGGVDTLNQMCSVMTCSRKTNRW
PMALLYGMINIACINSFIIYSHNVSSKGEKVQSRKKFMRNLYMSLTSSFMRKRLEAP
TLKRYLRDNISNILPKEVPGTSDDSTEEPVMKKRTYCTYCPSKIRRKANASCKKCK
KVICREHNIDMCQSCFagggSTEEPVMKKRTYCTYCPSKIRRKANASCKKCKKVICREHN
IDMCQSCF.
(SEQ ID NO: 93)
MAPKKKRKVGGGGSSLDDEHILSALLQSDDELVGEDSDSEVSDHVSEDDVQSDTEEA
FIDEVHEVQPTSSGSEILDEQNVIEQPGSSLASNRILTLPQRTIRGGGGGSERPYACPVE
SCDRRFSRSDELTRHIRIHTGQKPFQCRICMRNFSRSDHLTTHIRTHTGEKPFACDICGRKFAR
SDERKRHTKIHLRQKDGGGGSNKHCWSTSKSTRRSRVSALNIVRSQRGPTRMCRNIYD
PLLCFKLFFTDEIISEIVKWTNAEISLKRRESMTSATFRDTNEDEIYAFFGILVMTAV
RKDNHMSTDDLFDRSLSMVYVSVMSRDRFDFLIRCLRMDDKSIRPTLRENDVFTPV
RKIWDLFIHQCIQNYTPGAHLTIDEQLLGFRGRCPFRVYIPNKPSKYGIKILMMCDS
GTKYMINGMPYLGRGTQTNGVPLGEYYVKELSKPVHGSCRNITCDNWFTSIPLAK
NLLQEPYKLTIVGTVRSNKREIPEVLKNSRSRPVGTSMFCFDGPLTLVSYKPKPAK
MVYLLSSCDEDASINESTGKPQMVMYYNQTKGGVDTLDQMCSVMTCSRKTNRWP
MALLYGMINIACINSFIIYSHNVSSKGEKVQSRKKFMRNLYMSLTSSFMRKRLEAPT
LKRYLRDNISNILPKEVPGTSDDSTEEPVMKKRTYCTYCPSKIRRKANASCKKCKK
VICREHNIDMCQSCFagggSTEEPVMKKRTYCTYCPSKIRRKANASCKKCKKVICREHNI
DMCQSCF.

### Nuclear Localization Signals

In some embodiments, the transposase domains and fusion proteins provided herein may comprise an in-frame nuclear localization sequence (NLS). Examples of transposases fused to a nuclear localization signal are disclosed in U.S. Pat. Nos. 6,218,185; 6,962,810, 8,399,643 and WO 2019/173636. In some embodiments, the NLS comprises the sequence of PKKKRKV (SEQ ID NO: 88). In certain aspects, the in-frame NLS is located upstream (N-terminal) of the transposase domain comprising an N-terminal deletion.

In general, the NLS is preferably located at the N-terminal end of a fusion protein. In some embodiments, the NLS is fused or linked to the N-terminus of a transposase domain. In some embodiments, the NLS is fused or linked to the N-terminus of a DNA targeting domain. In some embodiments, the NLS is fused or linked to the N-terminus of a PSD.

In certain aspects, the in-frame NLS is fused directly to the amino terminus of the transposase domain comprising an N-terminal deletion. In some embodiments, the NLS is attached to the N-terminus of a transposase domain comprising an N-terminal deletion via a linker (e.g., a GGGGS linker or a GGS linker).

In some embodiments, an initiator methionine is introduced before the NLS. In some embodiments, additional alanine residues are introduced before and/or after the NLS to ensure in-frame translation. As such, the numbering of the residues in SEQ ID NO: 1 begins at the 12^{th} residue of SEQ ID NO: 1 for the purpose of identifying deleted and mutated residues. In SEQ ID NOs: 71-73, which are the sequence of SPB and PBx version 1 and PBx version 2 comprising a second CRD domain, respectively, which do not comprise an NLS, the numbering of residues begins at the 1^{st} residue for the purpose of identifying deleted and mutated residues.

### Obligate Heterodimers

In another aspect, provided herein are obligate heterodimer transposases comprising two fusion proteins, each fusion protein comprising a DNA targeting domain and a transposase domain. In some embodiments, both fusion proteins comprise DNA targeting domains and the DNA targeting domains target and bind DNA sequences that are adjacent to the DNA sequence that is the insertion site targeted by the transposase. A DNA-targeting domain may be attached to the C-terminus or the N-terminus of the fusion protein.

Thus, in some embodiments, provided herein is a complex comprising (a) a first fusion protein comprising a first DNA targeting domain, a linker, and a first transposase domain; and (b) a second fusion protein comprising a DNA targeting domain, a linker, and a second transposase domain; wherein the first DNA targeting domain and the second DNA targeting domain are different; wherein the transposase domains of the first fusion protein and the transpose domains of the second fusion protein have opposing charge that permits the two fusion proteins to form a complex.

In some embodiments, provided herein is a complex comprising (a) a first fusion protein comprising, in N-terminal to C-terminal order: a first NLS, a first DNA targeting domain, and a first transposase domain comprising an N-terminal deletion; and (b) a second fusion protein comprising in N-terminal to C-terminal order: a second NLS, a second DNA targeting domain, and a second transposase domain comprising an N-terminal deletion; wherein the transposase domains of the first fusion protein and the transpose domains of the second fusion protein have opposing charge that permits the two fusion proteins to form a complex. In some embodiments, the first and/or second transposase domains are SPB domains. In some embodiments, the first and/or second transposase domains are PBx transposase domains. In some embodiments, the first and/or second transposase domain comprises an N-terminal deletion of 83, 84, 85, 86, 87, 88, 89, 90, 91, 21, 93, 94, 95, 96, 97, 98, 99, 100, 101, 102, or 103 amino acids. In some embodiments, the first and second transposase domains comprise the sequence of SEQ ID NO: 38 or 59. In some embodiment, the first and/or second DNA targeting domain comprises one or more Zinc Finger Motif. In some embodiment, the first and/or second DNA targeting domain comprises one Zinc Finger Motif. In some embodiment, the first and/or second DNA targeting domain comprises two Zinc Finger Motifs. In some embodiment, the first and/or second DNA targeting domain comprises three Zinc Finger Motifs. In some embodiments, the first and/or second DNA targeting domain comprises the sequence of SEQ ID NO: 74. In some embodiment, the first and/or second DNA targeting domain comprises one or more TAL motifs.

In some embodiments, provided herein is a complex comprising (a) a first fusion protein comprising, in N-terminal to C-terminal order: a first NLS, a first PSD, a first DNA targeting domain, and a first transposase domain comprising an N-terminal deletion; and (b) a second fusion protein comprising in N-terminal to C-terminal order: a second NLS, a second PSD, a second DNA targeting domain, and a second transposase domain comprising an N-terminal deletion; wherein the transposase domains of the first fusion protein and the transpose domains of the second fusion protein have opposing charge that permits the two fusion proteins to form a complex. In some embodiments, the first and/or second transposase domains are SPB domains. In some embodiments, the first and/or second transposase domains are PBx transposase domains. In some embodiments, the first and second transposase domains comprise the sequence of SEQ ID NO: 38 or 59. In some embodiments, the first and/or second PSD comprises the sequence of SEQ ID NO: 91. In some embodiment, the first and/or second DNA targeting domain comprises three Zinc Fingers Motifs. In some embodiments, the first and/or second DNA targeting domain comprises the sequence of SEQ ID NO: 74. In some embodiment, the first and/or second DNA targeting domain comprises TAL motifs.

In some embodiments, provided herein is a complex comprising (a) a first fusion protein comprising, in N-terminal to C-terminal order: a first NLS, and a first transposase domain comprising the sequence of SEQ ID NOs: 71-73; and (b) a second fusion protein comprising in N-terminal to C-terminal order: a second NLS, and a second transposase domain comprising the sequence of SEQ ID NO: 71-73; wherein the first and the second transposase domain comprise a DNA targeting domain, and wherein the transposase domains of the first fusion protein and the transpose domains of the second fusion protein have opposing charge that permits the two fusion proteins to form a complex. In some embodiments, the first and/or second transposase domains are SPB domains. In some embodiments, the first and/or second transposase domains are PBx transposase domains. In some embodiments the first and second transposase domains comprise the sequence of SEQ ID NOs: 71-73. In some embodiments, the first and/or second PSD comprises the sequence of SEQ ID NO: 91. In some embodiment, the first and/or second DNA targeting domain comprises three Zinc Fingers Motifs. In some embodiments, the first and/or second DNA targeting domain comprises the sequence of SEQ ID NO: 74. In some embodiment, the first and/or second DNA targeting domain comprises TAL motifs. In some embodiments the first DNA targeting domain replaces the 83^{rd}, 84^{th}, 85^{th}, 86^{th}, 87^{th}, 88^{th}, 89^{th}, 90^{th}, 91^{st}, 92^{nd}, 93^{rd}, 94^{th}, 95^{th}, 96^{th}, 97^{th}, 98^{th}, 99^{th}, 100^{th}, 101^{st}, 102^{nd} or 103^{rd} residue of the first transposase domain of SEQ ID NOs: 71-73. In some embodiments, the second DNA targeting domain replaces the 83^{rd}, 84^{th}, 85^{th}, 86^{th}, 87^{th}, 88^{th}, 89^{th}, 90^{th}, 91^{st}, 92^{nd}, 93^{rd}, 94^{th}, 95^{th}, 96^{th}, 97^{th}, 98^{th}, 99^{th}, 100^{th}, 101^{st}, 102^{nd}, or 103^{rd} residue of the second transposase domain of SEQ ID NOs: 71-73.

In some embodiments, provided herein is a complex comprising (a) a first fusion protein comprising a first transposase domain, a linker, and a first DNA targeting domain, wherein the transposase domain of the first fusion protein comprise the same amino acid sequence set forth in any one of SEQ ID NOs: 28-69; and (b) a second fusion protein comprising a first transposase domain, a linker, and a second DNA targeting domain, wherein the transposase domain of the second fusion protein comprise the same amino acid sequence set forth in any one of SEQ ID NOs: 28-69.

In another aspect, provided herein are fusion proteins comprising a transposase domain that can form obligate heterodimers with another fusion protein comprising a transposase domain. Without wishing to be bound by theory, it is believed that two such fusion proteins assemble into an obligate heterodimer structure held together through a combination of charge interactions, hydrogen bonds, pi-cation pairs, and hydrophobic interactions. Such an obligate heterodimer structure is referred to herein as an “obligate heterodimer transposase.” Thus, each obligate heterodimer transposase comprises two transposase domains. In some embodiments, two fusion proteins provided herein form a complex, said complex comprising (a) a first fusion protein comprising a transposase domain; and (b) a second fusion protein comprising a transposase domain wherein the transposase domains of the first fusion protein and the transpose domains of the second fusion protein have opposing charge that permits the two fusion proteins to form a complex.

By introducing charged residues into the amino acids that contribute to the dimerization with a second fusion protein, it is possible to design pairs of fusion proteins that can only associate with each other into an obligate heterodimer in a predetermined configuration. By introducing mutations that only allow for one configuration of the obligate heterodimer, it becomes feasible to introduce DNA targeting domains into the fusion proteins, thus increasing specificity of the transposase domains. Introducing DNA targeting domains into fusion proteins that can dimerize in any configuration, including homodimerization, would lead to two copies of the same DNA targeting domains being present in a dimer transposase. However, only one of those DNA targeting domains would interact with the DNA, leaving the other to potentially sterically hinder the transposase-DNA interaction. Any suitable DNA targeting domain described herein or known in the art may be used in the fusion proteins described herein.

A person of skill in the art will readily be able to determine mutations in the transposase domains that confer a positive or negative charge. In the case of a fusion protein comprising a transposase domain, the crystal structure published in Chen et al. (Nat Commun 11, 3446 (2020)) may be used to identify residue pairs in the transposase domains that are in close proximity in the dimer formed by two such fusion proteins. Changing the charge of such residue pairs to create a positively charged transposase domain and a negatively charged transposase domain can be accomplished using standard techniques, such as site-directed mutagenesis.

For example, one or more of M185, R189, K190, D191, H193, M194, D198, D201, 5203, L204, 5205, V207, K500, R504, K575, K576, R583, N586, 1587, D588, M589, C593, and/or F594 may be mutated in an SPB transposase domain (e.g., the SPB set forth in SEQ ID NO: 1 or 73, with numbering beginning at the 12^{th} residue of SEQ ID NO: 1 and at the 1^{st} residue of SEQ ID NO: 73) to generate an SPB− or an SPB+ transposase domain. Similarly, one or more of M185, R189, K190, D191, H193, M194, D198, D201, S203, L204, S205, V207, K500, R504, K575, K576, R583, N586, 1587, D588, M589, C593, and/or F594 may be mutated in a PBx transposase domain (e.g., the PBx transposase domain of SEQ ID NOs: 70-72 to generate a PBx− or a PBx+ transposase domain.

To accomplish formation of an obligate heterodimer, pairs of mutations may be introduced into fusion proteins or transposase domains to generate positive and negatively charged fusion proteins or transposase domains which can then interact for form a heterodimer. In some embodiments, the residue pair being mutated is one set forth in Table 3. For example, one or more of the mutations listed in the column labeled “Protein 1” may be introduced into a first SPB or PBx domain and the corresponding mutation or mutations listed in the column labeled “Protein 2” may be introduced into a second SPB or PBs domain. In some embodiments, the members of a residue pair are mutated to have opposing charges.

TABLE 3
Exemplary Residue Pairs; numbering begins at residue 12
of SEQ ID NO: 1 and the 1^{st} residue of SEQ ID NO: 73.

| Protein 1 | Protein 2 | Protein 1 | Protein 2 | Protein 1 | Protein 2 |
|---|---|---|---|---|---|
| M185 | L204 | D201 | R504 | R583 | D588 |
| R189 | R189 | S203 | R504 | N586 | D588 |
| R189 | D191 | L204 | R189 | I587 | R583 |
| R189 | M194 | L204 | L204 | I587 | I587 |
| R189 | L204 | L204 | S205 | D588 | I587 |
| K190 | K190 | L204 | R504 | D588 | D588 |
| K190 | H193 | S205 | L204 | D588 | M589 |
| K190 | M194 | V207 | S203 | M589 | M589 |
| D191 | R189 | V207 | L204 | M589 | F594 |
| H193 | K190 | K500 | D198 | C593 | M589 |
| M194 | R189 | R504 | D201 | F594 | K575 |
| M194 | K190 | K575 | F594 | F594 | K576 |
| D198 | K500 | K576 | F594 | F594 | M589 |

To introduce a positive charge, amino acids with uncharged side chains, such as methionine, or amino acids with a negatively charged side chain, such as aspartic acid, may be changed to positively charged amino acids, such as lysine or arginine. To introduce a negative charge, amino acids with positively charged side chains, such as arginine or lysine, or amino acids with hydrophobic side chains, such as leucine, may be changed to negatively charged amino acids, such as aspartic acid or glutamic acid.

In certain embodiments, one or more of the following mutations is/are introduced into one or both SPB transposase domains (e.g., the SPB set forth in SEQ ID NO: 1 or 73, with numbering beginning at the 12^{th} residue of SEQ ID NO: 1 and at the 1^{st} residue of SEQ ID NO: 73) of a fusion protein provided herein to generate an SPB+ fusion protein: M185R, M185K, D197K, D197R, D198K, D198R, D201K, and D201R. In some embodiments, an SPB+ transposase domain comprises an M185R mutation and a D198K mutation. In some embodiments, an SPB+ transposase domain comprises an M185R mutation and a D201R mutation. In some embodiments, an SPB+ transposase domain comprises a D197K mutation and a D201R mutation. In some embodiments, an SPB+ transposase domain comprises a D198K mutation and a D201R mutation. In some embodiments, an SPB+ transposase domain comprises an M185R mutation, a D198K mutation, and a D201R mutation.

In certain embodiments, one or more of the following mutations is/are introduced into one or both PBx transposase domains (e.g., the PBx transposase domain of SEQ ID NOs:70-72) of a fusion protein provided herein to generate an PBx+ fusion protein: M185R, M185K, D197K, D197R, D198K, D198R, D201K, and D201R. In some embodiments, an PBx+ transposase domain comprises an M185R mutation and a D198K mutation. In some embodiments, an PBx+ transposase domain comprises an M185R mutation and a D201R mutation. In some embodiments, an PBx+ transposase domain comprises a D197K mutation and a D201R mutation. In some embodiments, an PBx+ transposase domain comprises a D198K mutation and a D201R mutation. In some embodiments, an PBx+ transposase domain comprises an M185R mutation, a D198K mutation, and a D201R mutation.

In certain embodiments, one or more of the following mutations is/are introduced into one or both SPB transposase domains (e.g., the SPB set forth in SEQ ID NO: 1 or 73, with numbering beginning at the 12^{th} residue of SEQ ID NO: 1 and at the 1^{st} residue of SEQ ID NO: 73) of a fusion protein provided herein to generate an SPB− fusion protein: L204D, L204E, K500D, K500E, R504E, and R504D. In some embodiments, an SPB− transposase domain comprises an L204E mutation and a K500D mutation. In some embodiments, an SPB− transposase domain comprises an L204E mutation and an R504D mutation. In some embodiments, an SPB− transposase domain comprises a K500 mutation and an R504D mutation. In some embodiments, an SPB− transposase domain comprises an L204E mutation, a K500D mutation, and an R504D mutation.

In certain embodiments, one or more of the following mutations is/are introduced into one or both PBx transposase (e.g., the PBx transposase domain of SEQ ID NOs: 70-72) of a fusion protein provided herein to generate a PBx− fusion protein: L204D, L204E, K500D, K500E, R504E, and R504D. In some embodiments, a PBx− transposase domain comprises an L204E mutation and a K500D mutation. In some embodiments, a PBx− transposase domain comprises an L204E mutation and an R504D mutation. In some embodiments, a PBx− transposase domain comprises a K500 mutation and an R504D mutation. In some embodiments, an PBx− transposase domain comprises an L204E mutation, a K500D mutation, and an R504D mutation.

The SPB+, SPB−, PBx+, and PBx− fusion proteins and transposase domains may further comprise the N-terminal deletions of the transposase domain described herein. Thus, in some embodiments, provided herein is an SPB+ fusion protein comprising a first and a second SPB+ transposase domain, wherein the SPB+ transposase domain comprises an N-terminal deletion of about 20 amino acids, about 40 amino acids, about 60 amino acids, about 80 amino acids, about 100 amino acids, or about 115 amino acids. In some embodiments, the SPB+ transposase domain comprises an N-terminal deletion of 83 amino acids. In some embodiments, the SPB+ transposase domain comprises an N-terminal deletion of 84 amino acids. In some embodiments, the SPB+ transposase domain comprises an N-terminal deletion of 85 amino acids. In some embodiments, the SPB+ transposase domain comprises an N-terminal deletion of 86 amino acids. In some embodiments, the SPB+ transposase domain comprises an N-terminal deletion of 87 amino acids. In some embodiments, the SPB+ transposase domain comprises an N-terminal deletion of 88 amino acids. In some embodiments, the SPB+ transposase domain comprises an N-terminal deletion of 89 amino acids. In some embodiments, the SPB+ transposase domain comprises an N-terminal deletion of 90 amino acids. In some embodiments, the SPB+ transposase domain comprises an N-terminal deletion of 91 amino acids. In some embodiments, the SPB+ transposase domain comprises an N-terminal deletion of 92 amino acids. In some embodiments, the SPB+ transposase domain comprises an N-terminal deletion of 93 amino acids. In some embodiments, the SPB+ transposase domain comprises an N-terminal deletion of 94 amino acids. In some embodiments, the SPB+ transposase domain comprises an N-terminal deletion of 95 amino acids. In some embodiments, the SPB+ transposase domain comprises an N-terminal deletion of 96 amino acids. In some embodiments, the SPB+ transposase domain comprises an N-terminal deletion of 97 amino acids. In some embodiments, the SPB+ transposase domain comprises an N-terminal deletion of 98 amino acids. In some embodiments, the SPB+ transposase domain comprises an N-terminal deletion of 99 amino acids. In some embodiments, the SPB+ transposase domain comprises an N-terminal deletion of 100 amino acids. In some embodiments, the SPB+ transposase domain comprises an N-terminal deletion of 101 amino acids. In some embodiments, the SPB+ transposase domain comprises an N-terminal deletion of 102 amino acids. In some embodiments, the SPB+ transposase domain comprises an N-terminal deletion of 103 amino acids.

In some embodiments, provided herein is an SPB− fusion protein comprising a SPB− transposase domain, wherein the SPB− transposase domain comprises an N-terminal deletion of about 20 amino acids, about 40 amino acids, about 60 amino acids, about 80 amino acids, about 81 amino acids, about 82 amino acids, about 83 amino acids, about 84 amino acids, about 85 amino acids, about 86 amino acids, about 87 amino acids, about 88 amino acids, about 89 amino acids, about 90 amino acids, about 91 amino acids, about 92 amino acids, about 93 amino acids, about 94 amino acids, about 95 amino acids, about 96 amino acids, about 97 amino acids, about 98 amino acids, about 99 amino acids, about 100 amino acids, about 101 amino acids, about 102 amino acids, about 103 amino acids, or about 115 amino acids. In some embodiments, the SPB− transposase domain comprises an N-terminal deletion of 83 amino acids. In some embodiments, the SPB− transposase domain comprises an N-terminal deletion of 84 amino acids. In some embodiments, the SPB− transposase domain comprises an N-terminal deletion of 85 amino acids. In some embodiments, the SPB− transposase domain comprises an N-terminal deletion of 86 amino acids. In some embodiments, the SPB− transposase domain comprises an N-terminal deletion of 87 amino acids. In some embodiments, the SPB− transposase domain comprises an N-terminal deletion of 88 amino acids. In some embodiments, the SPB− transposase domain comprises an N-terminal deletion of 89 amino acids. In some embodiments, the SPB− transposase domain comprises an N-terminal deletion of 90 amino acids. In some embodiments, the SPB− transposase domain comprises an N-terminal deletion of 91 amino acids. In some embodiments, the SPB− transposase domain comprises an N-terminal deletion of 92 amino acids. In some embodiments, the SPB− transposase domain comprises an N-terminal deletion of 93 amino acids. In some embodiments, the SPB− transposase domain comprises an N-terminal deletion of 94 amino acids. In some embodiments, the SPB− transposase domain comprises an N-terminal deletion of 95 amino acids. In some embodiments, the SPB− transposase domain comprises an N-terminal deletion of 96 amino acids. In some embodiments, the SPB− transposase domain comprises an N-terminal deletion of 97 amino acids. In some embodiments, the SPB− transposase domain comprises an N-terminal deletion of 98 amino acids. In some embodiments, the SPB− transposase domain comprises an N-terminal deletion of 99 amino acids. In some embodiments, the SPB− transposase domain comprises an N-terminal deletion of 100 amino acids. In some embodiments, the SPB− transposase domain comprises an N-terminal deletion of 101 amino acids. In some embodiments, the SPB− transposase domain comprises an N-terminal deletion of 102 amino acids. In some embodiments, the SPB− transposase domain comprises an N-terminal deletion of 103 amino acids.

In some embodiments, provided herein is a PBx+ fusion protein comprising a PBx+ transposase domain, wherein the PBx+ transposase domain comprises an N-terminal deletion of about 20 amino acids, about 40 amino acids, about 60 amino acids, about 80 amino acids, about 100 amino acids, or about 115 amino acids. In some embodiments, the PBx+ transposase domain comprises an N-terminal deletion of 83 amino acids. In some embodiments, the PBx+ transposase domain comprises an N-terminal deletion of 84 amino acids. In some embodiments, the PBx+ transposase domain comprises an N-terminal deletion of 85 amino acids. In some embodiments, the PBx+ transposase domain comprises an N-terminal deletion of 86 amino acids. In some embodiments, the PBx+ transposase domain comprises an N-terminal deletion of 87 amino acids. In some embodiments, the PBx+ transposase domain comprises an N-terminal deletion of 88 amino acids. In some embodiments, the PBx+ transposase domain comprises an N-terminal deletion of 89 amino acids. In some embodiments, the PBx+ transposase domain comprises an N-terminal deletion of 90 amino acids. In some embodiments, the PBx+ transposase domain comprises an N-terminal deletion of 91 amino acids. In some embodiments, the PBx+ transposase domain comprises an N-terminal deletion of 92 amino acids. In some embodiments, the PBx+ transposase domain comprises an N-terminal deletion of 93 amino acids. In some embodiments, the PBx+ transposase domain comprises an N-terminal deletion of 94 amino acids. In some embodiments, the PBx+ transposase domain comprises an N-terminal deletion of 95 amino acids. In some embodiments, the PBx+ transposase domain comprises an N-terminal deletion of 96 amino acids. In some embodiments, the PBx+ transposase domain comprises an N-terminal deletion of 97 amino acids. In some embodiments, the PBx+ transposase domain comprises an N-terminal deletion of 98 amino acids. In some embodiments, the PBx+ transposase domain comprises an N-terminal deletion of 99 amino acids. In some embodiments, the PBx+ transposase domain comprises an N-terminal deletion of 100 amino acids. In some embodiments, the PBx+ transposase domain comprises an N-terminal deletion of 101 amino acids. In some embodiments, the PBx+ transposase domain comprises an N-terminal deletion of 102 amino acids. In some embodiments, the PBx+ transposase domain comprises an N-terminal deletion of 103 amino acids.

In some embodiments, provided herein is a PBx− fusion protein comprising a PBx− transposase domain, wherein the PBx− transposase domain comprises an N-terminal deletion of about 20 amino acids, about 40 amino acids, about 60 amino acids, about 80 amino acids, about 81 amino acids, about 82 amino acids, about 83 amino acids, about 84 amino acids, about 85 amino acids, about 86 amino acids, about 87 amino acids, about 88 amino acids, about 89 amino acids, about 90 amino acids, about 91 amino acids, about 92 amino acids, about 93 amino acids, about 94 amino acids, about 95 amino acids, about 96 amino acids, about 97 amino acids, about 98 amino acids, about 99 amino acids, about 100 amino acids, about 101 amino acids, about 102 amino acids, about 103 amino acids, or about 115 amino acids. In some embodiments, the PBx− transposase domain comprises an N-terminal deletion of 83 amino acids. In some embodiments, the PBx− transposase domain comprises an N-terminal deletion of 84 amino acids. In some embodiments, the PBx− transposase domain comprises an N-terminal deletion of 85 amino acids. In some embodiments, the PBx− transposase domain comprises an N-terminal deletion of 86 amino acids. In some embodiments, the PBx− transposase domain comprises an N-terminal deletion of 87 amino acids. In some embodiments, the PBx− transposase domain comprises an N-terminal deletion of 88 amino acids. In some embodiments, the PBx− transposase domain comprises an N-terminal deletion of 89 amino acids. In some embodiments, the PBx− transposase domain comprises an N-terminal deletion of 90 amino acids. In some embodiments, the PBx− transposase domain comprises an N-terminal deletion of 91 amino acids. In some embodiments, the PBx− transposase domain comprises an N-terminal deletion of 92 amino acids. In some embodiments, the PBx− transposase domain comprises an N-terminal deletion of 93 amino acids. In some embodiments, the PBx− transposase domain comprises an N-terminal deletion of 94 amino acids. In some embodiments, the PBx− transposase domain comprises an N-terminal deletion of 95 amino acids. In some embodiments, the PBx− transposase domain comprises an N-terminal deletion of 96 amino acids. In some embodiments, the PBx− transposase domain comprises an N-terminal deletion of 97 amino acids. In some embodiments, the PBx− transposase domain comprises an N-terminal deletion of 98 amino acids. In some embodiments, the PBx− transposase domain comprises an N-terminal deletion of 99 amino acids. In some embodiments, the PBx− transposase domain comprises an N-terminal deletion of 100 amino acids. In some embodiments, the PBx− transposase domain comprises an N-terminal deletion of 101 amino acids. In some embodiments, the PBx− transposase domain comprises an N-terminal deletion of 102 amino acids. In some embodiments, the PBx− transposase domain comprises an N-terminal deletion of 103 amino acids.

### Transposons Comprising Symmetrical Inverted Terminal Repeat Sequences (ITRs)

Also provided herein are transposons comprising left end (LE) and right end (RE) symmetrical ITRs described herein.

SPB transposase binds asymmetrically to the 35 bp LE ITR (SEQ ID NO: 4) and 63 bp RE ITR (SEQ ID NO: 5), whereas a dual CRD SPB binds symmetrically as a dimer to transposons with the LE ITR sequence on both ends of the transposon. Transposons comprising a second copy of the 35 bp LE ITR in place of the RE ITR are required for recognition of transposons using piggyBac transposases comprising dual CRD domains for wild type ITRs are poorly recognized (see Example 3). An exemplary symmetrical RE ITR is referred to as symmetrical ITR 0 bp (SEQ ID NO: 6). An exemplary symmetrical RE ITR comprising the DDBD binding site of the RE ITR is referred to as symmetrical ITR SNP (SEQ ID NO: 98).

In some embodiments, the transposon symmetrical RE ITR comprises additional base pairs within the RE ITR between the 10 bp DDBD binding sequence (SEQ ID NO: 11) and the CRD binding sequence. In some embodiments, the symmetrical ITRs each comprise one additional base pair: CCCTAGAAAGATAGTCATGCGTAAAATTGACGCATG (SEQ ID NO: 7). In some embodiments, the symmetrical ITRs each comprise two additional base pairs: CCCTAGAAAGATAGTCATTGCGTAAAATTGACGCATG (SEQ ID NO: 8). In some embodiments, the symmetrical ITRs each comprise three additional base pairs: CCCTAGAAAGATAGTCATATGCGTAAAATTGACGCATG (SEQ ID NO: 9).

In some embodiments, the transposon symmetrical RE ITR comprises additional base pairs within the RE ITR between the 10 bp DDBD binding sequence (SEQ ID NO: 12) and the CRD binding sequence In some embodiments, the symmetrical ITRs each comprise one additional base pairs for RE ITR pairs: CCCTAGAAAGATAATCATGCGTAAAATTGACGCATG (SEQ ID NO: 21). In some embodiments, the symmetrical ITRs each comprise two additional base pairs for RE ITR pairs: CCCTAGAAAGATAATCATTGCGTAAAATTGACGCATG (SEQ ID NO: 98). In some embodiments, the symmetrical ITRs each comprise three additional base pairs for RE ITR pairs: CCCTAGAAAGATAATCATATGCGTAAAATTGACGCATG (SEQ ID NO: 10).

Transposons comprising a LE ITR and one of the RE ITRs comprising additional base pairs are capable of being transposed and integrated by the SPB and PBx transposases comprising dual CRD domains as well as site-specific transposition using DNA binding domain fusion proteins.

### Nucleic Acids

Also provided herein are polynucleotides comprising nucleic acid sequences encoding the fusion proteins described herein. In some embodiments, the polynucleotides are isolated.

The isolated polynucleotides of the disclosure can be made using (a) recombinant methods, (b) synthetic techniques, (c) purification techniques, and/or (d) combinations thereof, as well-known in the art.

Methods of constructing nucleic acids encoding the transposase domains comprising an N-terminal deletion described herein are well known in the art or described herein, for example, PCR-based mutagenesis.

The fusion of the present disclosure can be generated using any suitable method known in the art or described herein.

The isolated polynucleotides of this disclosure, such as RNA, cDNA, genomic DNA, or any combination thereof, can be obtained from biological sources using any number of cloning methodologies known to those of skill in the art. In some aspects, oligonucleotide probes that selectively hybridize, under stringent conditions, to the polynucleotides of the present disclosure are used to identify the desired sequence in a cDNA or genomic DNA library.

Methods of amplification of RNA or DNA are well known in the art and can be used according to the disclosure without undue experimentation, based on the teaching and guidance presented herein. Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

The polynucleotides of the disclosure can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Expression Vectors and Host Cells

The disclosure also relates to vectors that include polynucleotides of the disclosure, host cells that are genetically engineered with the recombinant vectors, and the production of at least one protein scaffold by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. In some embodiments, the promoter is an EF-1α promoter. The expression constructs will further contain sites for transcription initiation, termination and, in the transcribed region, a ribosome binding site for translation. The coding portion of the mature transcripts expressed by the constructs will preferably include a translation initiating codon (e.g., ATG) at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, ampicillin, zeocin (Sh bla gene), puromycin (pac gene), hygromycin B (hygB gene), G418/Geneticin (neo gene), DIFR (encoding Dihydrofolate Reductase and conferring resistance to Methotrexate), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotes (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors will preferably but optionally include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD19, CD271, CD34, CD22, CD20, CD33, CD52, or any combination thereof. Cell surface markers further include the suicide gene marker RQR8 (Philip B et al. Blood. 2014 Aug. 21; 124(8):1277-87).

Expression vectors will preferably but optionally include at least one selectable drug resistance marker for isolation of cells modified by the compositions and methods of the disclosure. Selectable drug resistance markers of the disclosure may comprise wild-type or mutant Neo, DHFR, TYMS, FRANCF, RAD51C, GCS, MDR1, ALDH1, NKX2.2, or any combination thereof.

Those of ordinary skill in the art are knowledgeable in the numerous expression systems available for expression of a nucleic acid encoding a protein of the disclosure. Alternatively, nucleic acids of the disclosure can be expressed in a host cell by turning on (by manipulation) in a host cell that contains endogenous DNA encoding a protein scaffold of the disclosure. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the protein scaffolds, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Agl4, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Agl4 cells (ATCC Accession Number CRL-1851). In a preferred aspect, the recombinant cell is a P3X63Ab8.653 or an SP2/0-Agl4 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present disclosure are known and/or available, for instance, from the American Type Culture Collection Catalogue of Cell Lines and Hybridomas (www.atcc.org) or other known or commercial sources.

When eukaryotic host cells are employed, polyadenylation or transcription terminator sequences are typically incorporated into the vector. An example of a terminator sequence is the polyadenylation sequence from the bovine growth hormone gene. In some embodiments, the polyA sequence is an SV40 polyA sequence.

Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

The plasmid constructs described herein may be used to deliver nucleic acids encoding the transposase domains or fusion proteins described herein to a cell.

The transposase domains and fusion proteins described herein may also be delivered to a cell using mRNA constructs. Thus, in one embodiment, provided herein is an mRNA sequence encoding a transposase domain or a fusion protein described herein. Such mRNA sequences may be delivered to a cell using a nanoparticle, for example, a lipid nanoparticle. Examples of lipid nanoparticles are described in, e.g., International Patent Applications No. PCT/US2021/055876, No. PCT/US2022/017570, U.S. Provisional Application No. 63/397,268, U.S. Provisional Application No. 63/301,855 and U.S. Provisional Application No. 63/348,614, each of which is incorporated herein by reference in its entirety for examples of lipid nanoparticles that may be used to deliver mRNA constructs encoding the fusion proteins or transposase domains described herein. An mRNA construct may also be delivered to a cell by electroporation or nucleofection. The mRNA may be capped or otherwise modified.

### Cells and Modified Cells

The dual CRD transposases and fusion proteins described herein may be used in conjunction with a transposon, including transposons comprising symmetrical ITRs, to modify cells. The transposon can be a piggyBac™ (PB) transposon. In some embodiments, when the transposon is a PB transposon, the transposase is a piggyBac™ (PB) transposase a piggyBac-like (PBL) transposase or a Super piggyBac™ (SPB) transposase. Non-limiting examples of PB transposons are described in detail in U.S. Pat. Nos. 6,218,182; 6,962,810; 8,399,643 and PCT Publication No. WO 2010/099296, each of which is incorporated herein by reference in its entirety for examples of transposons that may be used in connection with the transposases and methods described herein. The transposons can comprise a nucleic acid encoding a therapeutic protein or therapeutic agent. Examples of therapeutic proteins include those disclosed in PCT Publication No. WO 2019/173636 and PCT/US2019/049816.

Thus, provided herein are modified cells comprising one or more transposon and one or more dual CRD transposase or fusion proteins described herein. Cells and modified cells of the disclosure can be mammalian cells. Preferably, the cells and modified cells are human cells.

A cell modified using a dual CRD transposase or fusion protein described herein can be a germline cell or a somatic cell. Cells and modified cells of the disclosure can be immune cells, e.g., lymphoid progenitor cells, natural killer (NK) cells, T lymphocytes (T-cell), stem memory T cells (T_{SCM} cells), central memory T cells (T_{CM}), stem cell-like T cells, B lymphocytes (B-cells), antigen presenting cells (APCs), cytokine induced killer (CIK) cells, myeloid progenitor cells, neutrophils, basophils, eosinophils, monocytes, macrophages, platelets, erythrocytes, red blood cells (RBCs), megakaryocytes or osteoclasts. The modified cell can be differentiated, undifferentiated, or immortalized. The modified undifferentiated cell can be a stem cell. The modified undifferentiated cell can be an induced pluripotent stem cell. The modified cell can be a T cell, a hematopoietic stem cell, a natural killer cell, a macrophage, a dendritic cell, a monocyte, a megakaryocyte, or an osteoclast. The modified cell can be modified while the cell is quiescent, in an activated state, resting, in interphase, in prophase, in metaphase, in anaphase, or in telophase. The modified cell can be fresh, cryopreserved, bulk, sorted into sub-populations, from whole blood, from leukapheresis, or from an immortalized cell line. A detailed description for isolating cells from a leukapheresis product or blood is disclosed in in PCT Publication No. WO 2019/173636 and PCT/US2019/049816.

The methods of the disclosure can modify and/or produce a population of modified T cells, wherein at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between of the plurality of modified T cells in the population expresses one or more cell-surface marker(s) of a stem memory T cell (T_{SCM}) or a T_{SCM}-like cell; and wherein the one or more cell-surface marker(s) comprise CD45RA and CD62L. The cell-surface markers can comprise one or more of CD62L, CD45RA, CD28, CCR7, CD127, CD45RO, CD95, CD95 and IL-2Rβ. The cell-surface markers can comprise one or more of CD45RA, CD95, IL-2Rβ, CCR7, and CD62L.

The disclosure provides methods of expressing a CAR on the surface of a cell. The method comprises (a) obtaining a cell population; (b) contacting the cell population to a composition comprising a CAR or a sequence encoding the CAR, under conditions sufficient to transfer the CAR across a cell membrane of at least one cell in the cell population, thereby generating a modified cell population; (c) culturing the modified cell population under conditions suitable for integration of the sequence encoding the CAR; and (d) expanding and/or selecting at least one cell from the modified cell population that express the CAR on the cell surface. A more detailed description of methods for expressing a CAR on the surface of a cell is disclosed in PCT Publication No. WO 2019/049816 and PCT/US2019/049816.

The present disclosure provides a cell or a population of cells wherein the cell comprises a composition comprising (a) an inducible transgene construct, comprising a sequence encoding an inducible promoter and a sequence encoding a transgene, and (b) a receptor construct, comprising a sequence encoding a constitutive promoter and a sequence encoding an exogenous receptor, such as a CAR, wherein, upon integration of the construct of (a) and the construct of (b) into a genomic sequence of a cell, the exogenous receptor is expressed, and wherein the exogenous receptor, upon binding a ligand or antigen, transduces an intracellular signal that targets directly or indirectly the inducible promoter regulating expression of the inducible transgene (a) to modify gene expression.

The disclosure further provides a composition comprising the modified, expanded and selected cell population of the methods described herein.

The modified cells of disclosure (e.g., CAR T-cells) can be further modified to enhance their therapeutic potential. Alternatively, or in addition, the modified cells may be further modified to render them less sensitive to immunologic and/or metabolic checkpoints, for example by blocking and/or diluting specific checkpoint signals delivered to the cells (e.g., checkpoint inhibition) naturally, within the tumor immunosuppressive microenvironment.

The modified cells of disclosure (e.g., CAR T-cells) can be further modified to silence or reduce expression of (i) one or more gene(s) encoding receptor(s) of inhibitory checkpoint signals; (ii) one or more gene(s) encoding intracellular proteins involved in checkpoint signaling; (iii) one or more gene(s) encoding a transcription factor that hinders the efficacy of a therapy; (iv) one or more gene(s) encoding a cell death or cell apoptosis receptor; (v) one or more gene(s) encoding a metabolic sensing protein; (vi) one or more gene(s) encoding proteins that that confer sensitivity to a cancer therapy, including a monoclonal antibody; and/or (vii) one or more gene(s) encoding a growth advantage factor. Non-limiting examples of genes that may be modified to silence or reduce expression or to repress a function thereof include, but are not limited the exemplary inhibitory checkpoint signals, intracellular proteins, transcription factors, cell death or cell apoptosis receptors, metabolic sensing protein, proteins that that confer sensitivity to a cancer therapy and growth advantage factors that are disclosed in PCT Publication No. WO 2019/173636.

The modified cells of disclosure (e.g., CAR T-cells) can be further modified to express a modified/chimeric checkpoint receptor. The modified/chimeric checkpoint receptor can comprise a null receptor, decoy receptor or dominant negative receptor. Exemplary null, decoy, or dominant negative intracellular receptors/proteins include, but are not limited to, signaling components downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene. Non-limiting examples of cytokines, cytokine receptors, chemokines and chemokine receptors are disclosed in PCT Publication No. WO 2019/173636.

Genome modification can comprise introducing a nucleic acid sequence, transgene and/or a genomic editing construct into a cell ex vivo, in vivo, in vitro or in situ to stably integrate a nucleic acid sequence, transiently integrate a nucleic acid sequence, produce site-specific integration of a nucleic acid sequence, or produce a biased integration of a nucleic acid sequence. The nucleic acid sequence can be a transgene.

The stable chromosomal integration can be a random integration, a site-specific integration, or a biased integration. Without wishing to be bound by theory, it is believed that the addition of DNA binding domains to the transposases described herein improves the site-specificity of the transposases.

The site-specific integration can occur at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Non-limiting examples of potential genomic safe harbors include intronic sequences of the human albumin gene, the adeno-associated virus site 1 (AAVS1), a naturally occurring site of integration of AAV virus on chromosome 19, the site of the chemokine (C—C motif) receptor 5 (CCR5) gene and the site of the human ortholog of the mouse Rosa26 locus.

The site-specific transgene integration can occur at a site that disrupts expression of a target gene. Disruption of target gene expression can occur by site-specific integration at introns, exons, promoters, genetic elements, enhancers, suppressors, start codons, stop codons, and response elements. Non-limiting examples of target genes targeted by site-specific integration include TRAC, TRAB, PD1, any immunosuppressive gene, and genes involved in allo-rejection.

The site-specific transgene integration can occur at a site that results in enhanced expression of a target gene. Enhancement of target gene expression can occur by site-specific integration at introns, exons, promoters, genetic elements, enhancers, suppressors, start codons, stop codons, and response elements.

The site-specific transgene integration site can be a non-stable chromosomal insertion. The non-stable integration can be a transient non-chromosomal integration, a semi-stable non chromosomal integration, a semi-persistent non-chromosomal insertion, or a non-stable chromosomal insertion. The transient non-chromosomal insertion can be epi-chromosomal or cytoplasmic. In an aspect, the transient non-chromosomal insertion of a transgene does not integrate into a chromosome and the modified genetic material is not replicated during cell division.

The site-specific transgene integration site can be a modified binding site for the DNA targeting domain in a transposon domain, fusion protein, or tandem dimer described herein. For example, the TTAA target DNA integration site for SPB may be modified to insert flanking DNA binding sites for the DNA targeting domain comprising three Zinc Finger Motifs (e.g., a DNA targeting domain comprising or consisting of the sequence of SEQ ID NO: 74 or a sequence having at least 75%, at least 80%, at least 85%, at least 90%, at least 95%, at least 98%, or at least 99% identity thereto). For example, it is believed that a DNA targeting domain comprising three Zinc Finger Motifs encoded by SEQ ID NO:74 binds to the DNA sequence GCGTGGGCG. Therefore, the introduction of two copies of the target DNA sequence flanking the TTAA target integration site for SPB, is believed to improve site-specific integration of an SPB transposase domain comprising a DNA targeting domain comprising three Zinc Finger Motifs. The two copies of target sequence are in reverse (5′) and complement (3′) orientation.

In some embodiments, provided herein is a polynucleotide comprising, in 5′ to 3′ order, the reverse complement of the sequence of a target site for a DNA targeting domain, a first spacer, the TTAA target integration site for SPB, a second spacer, and the sequence of target site for a DNA targeting domain. In some embodiments, the first spacer and the second spacer have the same length. In some embodiments, the first and/or the second spacer are 3 bp in length. In some embodiments, the first and/or the second spacer are 4 bp in length. In some embodiments, the first and/or the second spacer are 5 bp in length. In some embodiments, the first and/or the second spacer are 6 bp in length. In some embodiments, the first and/or the second spacer are 7 bp in length. In some embodiments, the first and/or the second spacer are 8 bp in length. In some embodiments, the first and/or the second spacer are 9 bp in length. In some embodiments, the first and/or the second spacer are 10 bp in length.

Exemplary sequences of polynucleotides comprising, in 5′ to 3′ order, the reverse complement of the sequence of the target site for a DNA targeting domain comprising three Zinc Finger Motifs, a first spacer, the TTAA target integration site for SPB, a second spacer, and the sequence of the target site for the DNA targeting domain comprising three Zinc Finger Motifs are set forth in SEQ ID NOs: 94-97. The length of the first and second spacer in SEQ ID NOs: 94-97 is 8 bp, 7 bp, 6 bp, and 5 bp, respectively and the reverse and the complement of the target site for the DNA targeting domain is underlined and the TTAA sequence is shown in bold:

(SEQ ID NO: 94)
ACGCCCACGCTTACATCTTTAAAGATGTAAGCGTGGGCGT
(SEQ ID NO: 95)
ACGCCCACGCTACATCTTTAAAGATGTAGCGTGGGCGT
(SEQ ID NO: 96)
ACGCCCACGCTCATCTTTAAAGATGAGCGTGGGCGT
(SEQ ID NO: 97)
ACGCCCACGCTCTCTTTAAAGAGAGCGTGGGCGT

The modified target site may be introduced into a cell or a cell line to facilitate targeted genomic engineering. For example, a cell line which has been engineered to comprise a modified target site for an SPB or a PBx provided herein can be transfected with said SPB or PBx as well as a transposon comprising donor DNA such that the donor DNA is inserted at the modified target site. In some embodiments, the cell line is a T cell line. In some embodiments, the modified target sequence is introduced into a highly expressed genomic region. In a specific embodiment, provided herein is a cell line comprising stably integrated in its genomic sequence a nucleic acid sequence comprising, in 5′ to 3′ order, the reverse complement of the sequence of the target site for a DNA targeting domain comprising three Zinc Finger Motifs, a first spacer, the TTAA target integration site for SPB, a second spacer, and the sequence of the target site for the DNA targeting domain comprising three Zinc Finger Motifs. In some embodiments, the cell line comprises the sequence of any one of SEQ ID NOs: 94-97 stably integrated in its genome. In some embodiments, the cell is an in vitro cell, e.g., a cell in cell culture.

For DNA binding domains comprising TALENs, the target site is determined by the sequence of the TALENs. A person of skill in the art will be able to modify the TALEN sequences to achieve the desired target specificity.

The genome modification can be a non-stable chromosomal integration of a transgene. The integrated transgene can become silenced, removed, excised, or further modified.

In some embodiments, the transposase domains, fusion proteins and tandem dimer complexes provided herein have better transposase efficacy than their wildtype equivalents. Transposase activity may be measured by any suitable assay known in the art or described herein, for example, a Split GFP assay. For example, the transposase domains, fusion proteins and tandem dimer complexes provided herein may have comparable on-target genome integration activity to their wildtype counterparts, but have decreased off-target genome integration activity compared to their wildtype counterparts.

In some embodiments, a transposase domain comprising an N-terminal deletion and a DNA targeting domain provided herein has a ratio of on-target to off-target activity of at least 50-fold, at least about 100-fold, at least about 150-fold, at least about 200-fold, at least about 250-fold, at least about 300-fold, at least about 350-fold, at least about 400-fold, at least about 450-fold, at least about 500-fold, at least about 550-fold, at least about 600-fold, at least about 650-fold, at least about 700-fold, at least about 750-fold, at least about 800-fold, at least about 850-fold, at least about 900-fold, at least about 950-fold, or at least about 1000-fold compared to the wildtype transposase domain.

In some embodiments, a transposase domain comprising a DNA targeting domain inserted into the N-terminal region of the transposase domain provided herein has a ratio of on-target to off-target activity of at least 50-fold, at least about 100-fold, at least about 150-fold, at least about 200-fold, at least about 250-fold, at least about 300-fold, at least about 350-fold, at least about 400-fold, at least about 450-fold, at least about 500-fold, at least about 550-fold, at least about 600-fold, at least about 650-fold, at least about 700-fold, at least about 750-fold, at least about 800-fold, at least about 850-fold, at least about 900-fold, at least about 950-fold, or at least about 1000-fold compared to the wildtype transposase domain.

In certain embodiments, the modified cells are used therapeutically in adoptive cell therapy.

Adoptive cell compositions that are “universally” safe for administration to any patient (not just the patient from which they are derived) requires a significant reduction or elimination of alloreactivity. Towards this end, cells of the disclosure (e.g., allogenic cells) can be modified to interrupt expression or function of a T-cell Receptor (TCR) and/or a class of Major Histocompatibility Complex (MHC). The TCR mediates graft vs host (GvH) reactions whereas the MHC mediates host vs graft (HvG) reactions. In preferred aspects, any expression and/or function of the TCR is eliminated to prevent T-cell mediated GvH that could cause death to the subject. Thus, in a preferred aspect, the disclosure provides a pure TCR-negative allogeneic T-cell composition (e.g., each cell of the composition expresses at a level so low as to either be undetectable or non-existent).

Expression and/or function of MHC class I (MHC-I, specifically, HLA-A, HLA-B, and HLA-C) is reduced or eliminated to prevent HvG and, consequently, to improve engraftment of cells in a subject. Improved engraftment results in longer persistence of the cells, and, therefore, a larger therapeutic window for the subject. Specifically, expression and/or function of a structural element of MHC-I, Beta-2-Microglobulin (B2M), is reduced or eliminated. Non-limiting examples of guide RNAs (gRNAs) for targeting and deleting MHC activators are disclosed in PCT Application No. PCT/US2019/049816.

A detailed description of non-naturally occurring chimeric stimulatory receptors, genetic modifications of endogenous sequences encoding TCR-alpha (TCR-α), TCR-beta (TCR-β), and/or Beta-2-Microglobulin (β2M), and non-naturally occurring polypeptides comprising an HLA class I histocompatibility antigen, alpha chain E (HLA-E) polypeptide is disclosed in PCT Application No. PCT/US2019/049816.

Under normal conditions, full T-cell activation depends on the engagement of the TCR in conjunction with a second signal mediated by one or more co-stimulatory receptors (e.g., CD28, CD2, 4-1BBL) that boost the immune response. However, when the TCR is not present, T cell expansion is severely reduced when stimulated using standard activation/stimulation reagents, including agonist anti-CD3 mAb. Thus, the present disclosure provides a non-naturally occurring chimeric stimulatory receptor (CSR) comprising: (a) an ectodomain comprising a activation component, wherein the activation component is isolated or derived from a first protein; (b) a transmembrane domain; and (c) an endodomain comprising at least one signal transduction domain, wherein the at least one signal transduction domain is isolated or derived from a second protein; wherein the first protein and the second protein are not identical.

The activation component can comprise a portion of one or more of a component of a T-cell Receptor (TCR), a component of a TCR complex, a component of a TCR co-receptor, a component of a TCR co-stimulatory protein, a component of a TCR inhibitory protein, a cytokine receptor, and a chemokine receptor to which an agonist of the activation component binds. The activation component can comprise a CD2 extracellular domain or a portion thereof to which an agonist binds.

The signal transduction domain can comprise one or more of a component of a human signal transduction domain, T-cell Receptor (TCR), a component of a TCR complex, a component of a TCR co-receptor, a component of a TCR co-stimulatory protein, a component of a TCR inhibitory protein, a cytokine receptor, and a chemokine receptor. The signal transduction domain can comprise a CD3 protein or a portion thereof. The CD3 protein can comprise a CD3ζ protein or a portion thereof.

The endodomain can further comprise a cytoplasmic domain. The cytoplasmic domain can be isolated or derived from a third protein. The first protein and the third protein can be identical. The ectodomain can further comprise a signal peptide. The signal peptide can be derived from a fourth protein. The first protein and the fourth protein can be identical. The transmembrane domain can be isolated or derived from a fifth protein. The first protein and the fifth protein can be identical.

The present disclosure also provides a non-naturally occurring chimeric stimulatory receptor (CSR) wherein the ectodomain comprises a modification. The modification can comprise a mutation or a truncation of the amino acid sequence of the activation component or the first protein when compared to a wild type sequence of the activation component or the first protein. The mutation or a truncation of the amino acid sequence of the activation component can comprise a mutation or truncation of a CD2 extracellular domain or a portion thereof to which an agonist binds. The mutation or truncation of the CD2 extracellular domain can reduce or eliminate binding with naturally occurring CD58.

The present disclosure provides a nucleic acid sequence encoding any CSR disclosed herein. The present disclosure provides a transposon or a vector comprising a nucleic acid sequence encoding any CSR disclosed herein.

The present disclosure provides a cell comprising any CSR disclosed herein. The present disclosure provides a cell comprising a nucleic acid sequence encoding any CSR disclosed herein. The present disclosure provides a cell comprising a vector comprising a nucleic acid sequence encoding any CSR disclosed herein. The present disclosure provides a cell comprising a transposon comprising a nucleic acid sequence encoding any CSR disclosed herein.

In certain aspects, the cells of the present disclosure are modified to recombinantly express dihydrofolate reductase (DHFR), which advantageously renders cells resistant to methotrexate (MTX). The MTX resistant cells may be used in methods of treating a subject in need thereof in combination with subsequent MTX administration to eliminate activated T-cells and NK cells targeting the modified cells or therapeutic cells, thereby increasing the in vivo persistence and efficacy of the modified cells.

The present disclosure provides a composition comprising any CSR disclosed herein. The present disclosure provides a composition comprising a nucleic acid sequence encoding any CSR disclosed herein. The present disclosure provides a composition comprising a vector comprising a nucleic acid sequence encoding any CSR disclosed herein. The present disclosure provides a composition comprising a transposon comprising a nucleic acid sequence encoding any CSR disclosed herein. The present disclosure provides a composition comprising a modified cell disclosed herein or a composition comprising a plurality of modified cells disclosed herein.

Also provided herein are methods of site-specific gene integration. The dual CRD transposase domains and fusion proteins provided herein may be used to deliver a transgene to a cell and integrate the transgene into a target site. The target site may be, for example, a genomic safe harbor, i.e., a genomic sites where a transgene can be integrated in a manner that ensures that the transgene functions predictably and unlikely to cause detrimental changes in the gene expression profile of a cell. In some embodiments, the target site is a repetitive element, such as a LINE-1 or ALU sequence. Repetitive elements do not encode essential gene products, making it unlikely that that an insertion leads to detrimental changes in the gene expression profile of a cell. There may be one, two or more target sites within one repetitive element. In some embodiments, the target site is located within an intron (e.g., an intron of the PAH gene).

The site-specific integration may be used in vitro or in vivo. An example of an in vivo application is gene therapy, which involves the delivery of a transgene to the genomic DNA of a cell.

### Formulations, Dosages and Modes of Administration

The present disclosure provides formulations, dosages and methods for administration of the compositions and cells described herein. In one aspect, provided herein is a pharmaceutical composition comprising a tandem dimer transposase or a fusion protein described herein and a pharmaceutically acceptable carrier. In another aspect, provided herein is a pharmaceutical composition comprising a modified cell described herein and a pharmaceutically acceptable carrier.

The disclosed compositions and pharmaceutical compositions can comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the protein scaffold, fragment or variant composition as well known in the art or as described herein.

Non-limiting examples of pharmaceutical excipients and additives suitable for use include proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Non-limiting examples of carbohydrate excipients suitable for use include monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferably, the carbohydrate excipients are mannitol, trehalose, and/or raffinose.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate.

Additionally, the disclosed compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means. In preferred embodiments, a composition comprising a modified cell described herein is administered intravenously, e.g., by intravenous infusion.

A composition of the disclosure can be prepared for use for parenteral (subcutaneous, intramuscular or intravenous) or any other administration particularly in the form of liquid solutions or suspensions. For parenteral administration, a composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection or infusion can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

It can be desirable to deliver the disclosed compounds to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation, such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b), e.g., a zinc tannate salt. Additionally, the disclosed compounds or, preferably, a relatively insoluble salt, such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

### Methods of Treatment

In another aspect, provided herein are methods of treating a disease or disorder in a subject, the method comprising administering to the subject a composition comprising the modified cells described herein. The terms “subject” and “patient” are used interchangeably herein. In preferred embodiments, the patient is human.

The modified cells may be allogeneic or autologous to the patient. In some preferred embodiments, the modified cell is an allogeneic cell. In some embodiments, the modified cell is an autologous T-cell or a modified autologous CAR T-cell. In some preferred embodiments, the modified cell is an allogeneic T-cell or a modified allogeneic CAR T-cell.

In some embodiments, the disease or disorder treated in accordance with the methods described herein is a cancer. In some embodiments, a method of treatment described herein may delay cancer progression and/or reduce tumor burden.

In some embodiments, the disease or disorder treated in accordance with the methods described herein is an autoimmune disorder. In some embodiments, the autoimmune disease is autoimmune neutropenia, Guillain-Barre syndrome, epilepsy, autoimmune encephalitis, Isaacs' syndrome, nevus syndrome, pemphigus vulgaris, deciduous pemphigus, bullous pemphigoid, acquired epidermolysis bullosa, gestational pemphigoid, mucous membrane pemphigoid, antiphospholipid syndrome, autoimmune anemia, myasthenia gravis, autoimmune Graves' disease, thyroid eye disease (TED), Goodpasture syndrome, multiple sclerosis, rheumatoid arthritis, lupus, idiopathic thrombocytopenic purpura (ITP), warm autoimmune hemolytic anemia (WAIHA), chronic inflammatory demyelinating polyneuropathy (CIDP), lupus nephritis, or membranous nephropathy.

The dosage of a pharmaceutical composition to be administered to a subject can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired.

In aspects where the compositions to be administered to a subject in need thereof are modified cells as disclosed herein, between about 1×10³ and about 1×10⁴ cells; between about 1×10⁴ and about 1×10⁵ cells; between about 1×10⁵ and about 1×10⁶ cells; between about 1×10⁶ and about 1×10⁷ cells; between about 1×10⁷ and about 1×10⁸ cells; between about 1×10⁸ and about 1×10⁹ cells; between about 1×10⁹ and about 1×10¹⁰ cells, between about 1×10¹⁰ and about 1×10¹¹ cells, between about 1×10¹¹ and about 1×10¹² cells, between about 1×10¹² and about 1×10¹³ cells, between about 1×10¹³ and about 1×10¹⁴ cells, between about 1×10¹⁴ and about 1×10¹⁵ cells, between about 1×10¹⁵ and about 1×10¹⁶ cells, between about 1×10¹⁶ and about 1×10¹⁷ cells, between about 1×10¹⁷ and about 1×10¹⁸ cells, between about 1×10¹⁸ and about 1×10¹⁹ cells; or between about 1×10¹⁹ and about 1×10²⁰ cells may be administered. In some embodiments, the cells are administered at a dose of between about 5×10⁶ and about 25×10⁶ cells.

In other embodiments, the dosage of cells may depend on the body weight of the person, e.g., between about 1×10³ and about 1×10⁴ cells; between about 1×10⁴ and about 1×10⁵ cells; between about 1×10⁵ and about 1×10⁶ cells; between about 1×10⁶ and about 1×10⁷ cells; between about 1×10⁷ and about 1×10⁸ cells; between about 1×10⁸ and about 1×10⁹ cells; between about 1×10⁹ and about 1×10¹⁰ cells, between about 1×10¹⁰ and about 1×10¹¹ cells, between about 1×10¹¹ and about 1×10¹² cells, between about 1×10¹² and about 1×10¹³ cells, between about 1×10¹³ and about 1×10¹⁴ cells, between about 1×10¹⁴ and about 1×10¹⁵ cells, between about 1×10¹⁵ and about 1×10¹⁶ cells, between about 1×10¹⁶ and about 1×10¹⁷ cells, between about 1×10¹⁷ and about 1×10¹⁸ cells, between about 1×10¹⁸ and about 1×10¹⁹ cells; or between about 1×10¹⁹ and about 1×10²⁰ cells may be administered per kg body weight of the subject.

A more detailed description of pharmaceutically acceptable excipients, formulations, dosages and methods of administration of the disclosed compositions and pharmaceutical compositions is disclosed in PCT Publication No. WO 2019/049816.

The transposon domains and fusion proteins provided herein may be used to deliver a gene therapy. Gene therapy usually involves the delivery of a transgene to the genomic DNA of a cell. Usually, the transgene replaces a gene that is mutated or otherwise not expressed properly in the cell. The fusion proteins, transposase domains, and complexes described herein may be used to deliver a therapeutic transgene to a cell and integrate the transgene into a target site. In some embodiments, a method of treatment comprises introducing into the cell a fusion protein described herein and a transposon, wherein the transposon comprises, in 5′ to 3′ order: a 5′ITR, the transgene, and a 3′ ITR.

### Kits

In another aspect, provided herein is a kit comprising a cell line which has been engineered to comprise a modified target site for an SPB or a PBx provided herein within its genome, preferably in a highly expressed genomic region. The kit may further comprise a composition comprising one or more SPB or PBx transposase domains or fusion proteins described herein. In some embodiments, the cell line is a T cell line.

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof. In the context of nucleic acids, a promoter may be operatively linked to a nucleotide sequence encoding a transpose domain or fusion protein described herein, bringing the expression of the nucleotide sequence under the control of the promoter.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “promoter” refers to a synthetic or naturally derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157: 105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Table 4, Table 5 and Table 6 below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table 4.

### Table 4: Conservative Substitutions I

### Side Chain Characteristics Amino Acid

Aliphatic Non-polar G A P I L V F
   Polar—uncharged C S T M N Q
   Polar—charged D E K R
Aromatic H F W Y
Other N Q D E

Alternately, conservative amino acids can be grouped as described in Lehninger, (Biochemistry, Second Edition; Worth Publishers, Inc. NY, N.Y. (1975), pp. 71-77) as set forth in Table 5.

TABLE 5
Conservative Substitutions II

| | |
|---|---|
| Side Chain Characteristic | Amino Acid |

| | | |
|---|---|---|
| Non-polar (hydrophobic) | Aliphatic: | A L I V P |
| | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table 6.

TABLE 6
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, identity between two sequences may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. In some embodiments, the sequence identify is determined over the entire length of a sequence. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

In certain embodiments, if a sequence has a certain sequence identity (e.g., 75%, 80%, 85%, 90%, 95%, 98%, or 99%) to a certain SEQ ID NO, the sequence and the sequence of the SEQ ID NO have the same length. In certain embodiments, if a sequence has a certain sequence identity (e.g., 75%, 80%, 85%, 90%, 95%, 98%, or 99%) to a certain SEQ ID NO, the sequence and the sequence of the SEQ ID NO only differ due to conservative amino acid substitutions.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

### EXAMPLES

The Examples in this section are provided for illustration and are not intended to limit the invention.

### Example 1—Construction of a Super PiggyBac Transposase Comprising Tandem Cysteine Rich Domains (CRDs)

This example illustrates the construction of an exemplary Super piggyBac (SPB) transposase comprising dual C-terminal CRDs.

The amino acid sequence of an SPB transposase comprising an N-terminal nuclear localization sequence (NLS; SEQ ID NO: 1) was used to construct a SPB transposase sequence comprising an additional C-terminal CRD. Amino acid residues 542-594 of the SPB transposase (SEQ ID NO: 2), which comprises the SPB CRD domain, were joined to the C-terminus of the SPB transposase sequence using an AGGG linker (SEQ ID NO: 27) to generate a SPB Dual CRD transposase comprising an N-terminal NLS (SEQ ID NO: 3). The integration and excision activities of the SPB transposase comprising dual CRDs was compared to wild type SPB transposase using wild type and symmetrical inverted terminal repeat sequences (ITRs) as set forth in Example 3.

### Example 2—Construction of Transposons Comprising Symmetrical Inverted Terminal Repeat (ITRs) Variants

This example illustrates the construction of transposons comprising symmetrical ITRs for use with SPB transposases and site-specific SPB/PBx transposases comprising dual CRDs.

SPB transposase binds asymmetrically to the 35 bp LE ITR (SEQ ID NO: 4) and 63 bp RE ITR (SEQ ID NO: 5) of a transposon; however, a SPB transposase comprising dual CRDs binds symmetrically recognizing a transposon with LE ITR sequences on both ends of the transposon. Accordingly, the 63 bp RE ITR of the transposon was substituted with a second copy of the 35 bp LE ITR, referred to as Symmetrical ITR or Symmetrical ITR 0 bp (SEQ ID NO: 6). Given that upon ITR binding, the four CRD domains of the two dual CRD SPB dimers are all in close proximity, possible steric hinderance was examined by designing several versions of the symmetrical ITRs in which the CRD binding site is shifted away from the DDBD binding site by 1 bp, 2 bp, or 3 bp to create Symmetrical ITR 1 bp (SEQ ID NO: 7), Symmetrical ITR 2 bp (SEQ ID NO: 8), and Symmetrical ITR 3 bp (SEQ ID NO: 9). Since the LE ITR 10 bp DDBD binding site (SEQ ID NO: 11) has one base pair difference from the RE ITR 10 bp DDBD binding site (SEQ ID NO: 12), a second version of the Symmetrical ITR 3 bp version was constructed (Symmetrical ITR 3bpSNP; SEQ ID NO: 10) to test the sequence difference between LE and RE DDBD binding sites. The integration and excision activity of transposons comprising wild type or symmetrical ITRs were analyzed using a dual excision/integration luciferase reporter assay.

### Example 3—Methods for Measuring Excision and Integration Activities of SPB Transposase Comprising Dual CRD Domains and Transposons Comprising Symmetrical Inverted Terminal Repeat (ITRs) Variants

This example illustrates methods for measuring the excision and integration activities of SPB transposases comprising dual CRDs using transposons comprising wild type or symmetrical ITRs.

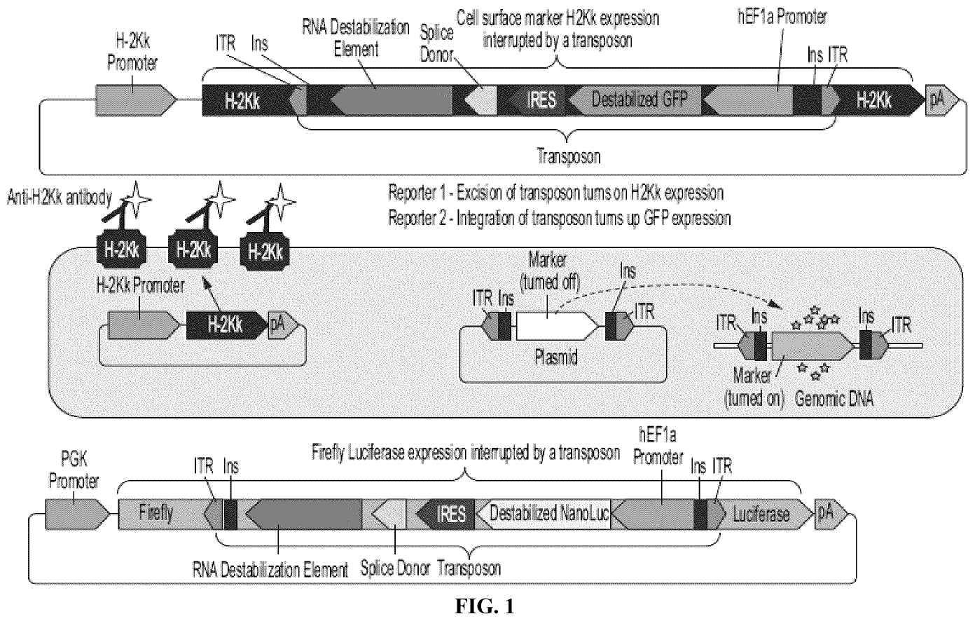

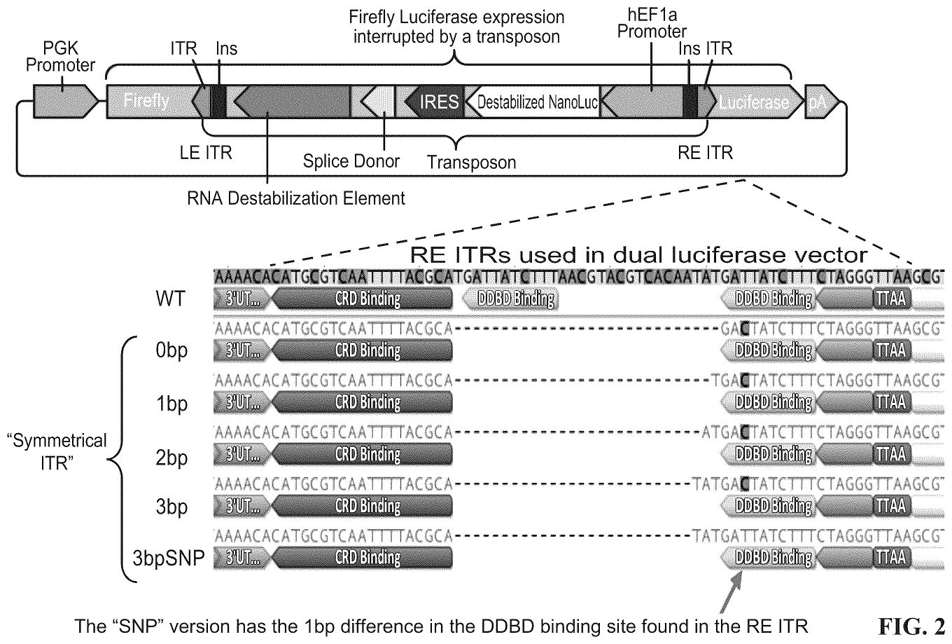

A dual excision/integration luciferase reporter (SEQ ID NO: 13) was employed to test the SPB transposase comprising dual CRDs of transposons comprising wild type or symmetrical ITRs (see FIG. 1). FIG. 1 shows a schematic depiction of the dual reporter plasmid design used to confirm the rates of excision and integration using each mutant transposon. Using an H-2kk GFP transposon reporter (Reporter 1), an increase in H2kk expression is observed if there is an increase in excision of the transposon. Using Reporter 2, an increase in GFP expression is observed if there is an increase in the integration of the transposon. In an alternative design of Reporter 2, an increase in Firefly luciferase expression is observed if there is an increase in excision of the transposon and an increase in NanoLuc is observed if there is an increase in the integration of the transposon. The WT 63 bp RE ITR sequence of the transposon in the reporter was replaced with each of the symmetrical ITRs (SEQ ID Nos: 6-10) as shown schematically in FIG. 2. K562 cells were nucleofected using 2035 of SF buffer and program FF-120 in accordance with the manufacturer's instructions. Each reaction contained 50 ng of the dual luciferase reporter and 450 ng of a dual CRD (SEQ ID NO: 3) or a SPB (SEQ ID NO: 1) expressing plasmid. As a negative control, each dual luciferase reporter was transfected without transposase. One day post transfection, luciferase signal was measured using Promega's dual luciferase reagents and a plate reader. The results are shown in Table 7.

TABLE 7

| | | Luciferase Signal | | | | | |
|---|---|---|---|---|---|---|---|
| | | Excision (Firefly Luc) | | | Integration (NanoLuc) | | |
| | | Replicate | Replicate | | Replicate | Replicate | |
| | | 1 | 2 | Average | 1 | 2 | Average |
| WT SPB | (WT) | 5672 | 5712 | 5692 | 168604 | 177976 | 173290 |
| | 0 bp | 193 | 64 | 129 | 3498 | 3851 | 3675 |
| | 1 bp | 125 | 96 | 111 | 7398 | 2436 | 4917 |
| | 2 bp | 50 | 94 | 72 | 13018 | 4629 | 8824 |
| | 3 bp | 25 | 106 | 66 | 4894 | 3520 | 4207 |
| | 3 bpSNP | 136 | 44 | 90 | 6446 | 4799 | 5623 |
| Dual CRD | (WT) | 3719 | 4069 | 3894 | 54642 | 49266 | 51954 |
| SPB | 0 bp | 806 | 662 | 734 | 16244 | 16182 | 16213 |
| | 1 bp | 245 | 393 | 319 | 7708 | 4468 | 6088 |
| | 2 bp | 1860 | 1031 | 1446 | 23107 | 19219 | 21163 |
| | 3 bp | 583 | 811 | 697 | 15175 | 10892 | 13034 |
| | 3 bpSNP | 1523 | 588 | 1056 | 28124 | 11963 | 20044 |
| No | 0 bp | 26 | 142 | 84 | 3541 | 1418 | 2480 |
| Transposase | 1 bp | 22 | 31 | 27 | 3484 | 2812 | 3148 |
| | 2 bp | 8 | 3 | 6 | 4442 | 2325 | 3384 |
| | 3 bp | 6 | 9 | 8 | 2676 | 4458 | 3567 |
| | Mock | 41 | | 41 | 56 | | 56 |

As shown in Table 7, a strong luciferase signal indicating transposon excision and integration was detected with SPB transposase in combination with the reporter harboring the wild type ITR sequences but not with those comprising any of the symmetrical ITR sequences. The SPB transposase comprising the dual CRD domains resulted in luciferase signal in combination with both the WT ITRs as well as all of the symmetrical ITR designs, although expression was detected at lower levels than SPB transposase. The symmetrical ITR 2 bp version (SEQ ID NO: 8) resulted in the highest signal amongst all the symmetrical ITR designs using the SPB transposase comprising the dual CRD domains. Additionally, both the LE ITR 10 bp DDBD binding site (SEQ ID NO: 11) and RE ITR 10 bp DDBD binding site (SEQ ID NO: 12) were functional for transposons comprising symmetrical ITRs as observed for the Symmetrical ITR 3 bp (SEQ ID NO: 9) and Symmetrical ITR 3bpSNP (SEQ ID NO: 10) samples. The reporter constructs in the absence of transposase resulted only in background levels of luciferase expression.

### Example 4—Construction and Analysis of TAL Array-PiggyBac Dual CRD Transposase (ss-PBx-CRD) Compositions (TAL-PBx-CRDs) Designed for Site-specific Transposition at Specific Genes

This Example illustrates the construction of TAL Array-Super piggyBac PBx transposase comprising dual CRD domains fusion protein compositions (TAL-PBx-CRD) that are useful in methods for achieving site-specific transposition at a specific target locus.

TAL-PBx transposases targeting LINE1 repetitive elements LINE1 L2 TAL-PBx (SEQ ID NO: 15) and LINE1 R2.2 TAL-PBx (SEQ ID NO: 16) comprising a +73 TAL C-terminus and an N-terminally deleted (delta 1-85) PBx fusion point (d85+73) were previously constructed (see, e.g., International Patent Application Publication No. WO 2023/060089). The two LINE1 TAL-PBx(d85+73) constructs were modified by appending an additional CRD domain to the C-terminus of the PBx transposase sequence. In one instance, LINE L2 TAL-PBx(d85+73) (SEQ ID NO: 15) and LINE R2.2 TAL-PBx(d85+73) (SEQ ID NO: 16) were appended with an AGGG linker (SEQ ID NO: 27) and amino acids 547-594 (SEQ ID NO: 2) comprising a SPB transposase CRD domain to create LINE L2 TAL-PBx(d85+73) Dual CRD (SEQ ID NO: 17) and LINE R2.2 TAL-PBx(d85+73) Dual CRD (SEQ ID NO: 18). Additionally, a second LINE1 left and right pair were constructed in which the LINE L2 TAL-PBx(d85+73) (SEQ ID NO: 15) and LINE R2.2 TAL-PBx(d85+73) (SEQ ID NO: 16) were appended directly with amino acids 542-594 (SEQ ID NO: 14) of SPB comprising a CRD domain to create LINE L2 TAL-PBx(d85+73) Dual CRD (SEQ ID NO:19) and LINE R2.2 TAL-PBx(d85+73) Dual CRD (SEQ ID NO: 20).

A disrupted GFP reporter system was used to test the LINE1 TAL-PBx− dual CRD transposases and in combination with transposons comprising symmetrical ITRs. Briefly, the reporter system contains an EF1a promoter (SEQ ID NO: 22) driving expression of a GFP CDS reporter (SEQ ID NO: 23) followed by a SV40 polyadenylation sequence (SEQ ID NO: 24). The GFP reporter is disrupted by a transposon at a TTAA sequence, breaking it into the first GFP section (SEQ ID NO: 25) and the second GFP section (SEQ ID NO: 26). Upon transposase mediated excision of the transposon, the reporter can be seamlessly repaired, resulting restoration of a full-length GFP CDS and subsequent GFP expression as a proxy for excision activity. The WT 63 bp RE ITR sequence in the reporter was replaced with the Symmetrical ITRs 0 bp (SEQ ID NO: 6) and Symmetrical ITR with a 1 bp spacer and the RE version of the 10 bp DDBD binding site, Symmetrical ITR 1bpSNP (SEQ ID NO: 21).

Each reporter construct was co-transfected into HEK293T cells with the TAL-ssSPB− CRD transposases. As controls, the TAL-ssSPB− CRD transposases were replaced with the original wild type ssSPB, by excision only PiggyBac (PBx) or by no transposase. Briefly 120,000 HEK293T cells were plated in 24 well plates in 500 μL of DMEM+10% FBS one day before transfection. A 50 ng amount of the pair of transposase-expressing vectors was combined with 450 ng of the reporter and transfected using 1 μL of JetPrime transfection reagent. Two days after transfection, the percentage of GFP positive cells was measured by flow cytometry. The results are shown in Table 8.

TABLE 8

| | Percentage of GFP Positive Cells (Transposon Excision) | | | | | | | | | | | |
|---|---|---|---|---|---|---|---|---|---|---|---|---|
| | Symmetric ITR 0 bp | | | | Symmetric ITR 1 bpSNP | | | | WT ITR | | | |
| Transposase | Rep1 | Rep2 | Rep3 | Avg | Rep1 | Rep2 | Rep3 | Avg | Rep1 | Rep2 | Rep3 | Avg |
| Single- | 66.4 | 63.6 | | 65.0 | 73.0 | 70.2 | 66.4 | 69.9 | 73.7 | 74.5 | 71.6 | 73.3 |
| CRD | | | | | | | | | | | | |
| Dual-CRD | 97.0 | 96.5 | | 96.8 | 90.2 | 96.6 | 92.2 | 93.0 | 81.5 | 87.3 | 86.1 | 85.0 |
| AGGG | | | | | | | | | | | | |
| 547-598 | | | | | | | | | | | | |
| Dual-CRD | 95.2 | 97.1 | | 96.2 | 90.5 | 95.9 | 96.2 | 94.2 | 80.1 | 88.1 | 85.4 | 84.5 |
| 542-598 | | | | | | | | | | | | |
| PBx | 90.2 | 90.5 | 92.2 | 91.0 | 90.3 | 92.0 | 91.5 | 91.3 | 97.4 | 99.6 | 99.5 | 98.8 |
| Transposon | 1.8 | 1.3 | 1.0 | 1.4 | 2.3 | 2.2 | 1.7 | 2.0 | 0.8 | 0.6 | 0.6 | 0.7 |
| Only | | | | | | | | | | | | |

As shown in Table 8, both TAL-PBx-CRD designs resulted in higher excision than TAL-PBx transposases comprising a single CRD version and exhibited excision activity on par with PBx transposase. Additionally, the PBx and the single CRD ssSPB transposases resulted in higher excision activity using the WT ITR reporter than those comprising the symmetrical ITR reporters; however, TAL-PBx versions comprising dual CRD domains resulted in higher excision for symmetrical ITR reporters than with the WT ITR reporter. The negative control with no transposase had background levels of GFP signal.

Genomic DNA was harvested from the transfected cells described above and site-specific integration of the transposon into the LINE1 target sites was quantified by ddPCR. The transposon integration events in the forward orientation per haploid genome are shown in Table 9.

TABLE 9

| | Transposon Integrations Per Haploid Genome | | | | | | | | | | | |
|---|---|---|---|---|---|---|---|---|---|---|---|---|
| | Symmetric ITR 0 bp | | | | Symmetric ITR 1 bpSNP | | | | WT ITR | | | |
| Transposase | Rep1 | Rep2 | Rep3 | Avg | Rep1 | Rep2 | Rep3 | Avg | Rep1 | Rep2 | Rep3 | Avg |
| Single- | 3.39 | 3.78 | 3.85 | 3.67 | 3.34 | 3.67 | 3.81 | 3.61 | 4.56 | 4.18 | 4.54 | 4.43 |
| CRD | | | | | | | | | | | | |
| Dual- | 10.50 | 12.80 | 12.60 | 11.97 | 9.52 | 10.50 | 9.22 | 9.75 | 6.71 | 8.62 | 7.89 | 7.74 |
| CRD | | | | | | | | | | | | |
| AGGG | | | | | | | | | | | | |
| 547-598 | | | | | | | | | | | | |
| Dual- | 10.10 | 12.00 | 11.80 | 11.30 | 10.70 | 13.60 | 12.30 | 12.20 | 5.59 | 7.74 | 8.39 | 7.24 |
| CRD | | | | | | | | | | | | |
| 542-598 | | | | | | | | | | | | |
| PBx | 0.00 | 0.00 | 0.00 | 0.00 | 0.00 | 0.00 | 0.00 | 0.00 | 0.00 | 0.00 | 0.00 | 0.00 |

As shown in Table 9, approximately three to five integration events per haploid genome were detected using a wild type TAL-PBx transposase comprising a single CRD, with slightly more integration events occurring for transposons comprising WT ITRs than with the symmetrical ITRs. A greater number of integrations was observed using TAL-PBx transposases comprising dual CRD domains resulting in six to nine transposon integrations per haploid genome with the WT ITRs and an increase to nine to thirteen transposon integrations per haploid genome using the symmetrical ITRs. The two versions of the TAL-PBx dual CRD fusion proteins resulted in a similar number of integrations.

### Example 5—Construction of a Super PiggyBac Transposase Comprising Tandem Cysteine Rich Domains (CRDs) and N-Terminal Deletion

This example illustrates the construction of an exemplary Super piggyBac (SPB) transposase comprising dual C-terminal CRDs as well as an N-terminal deletion (NTD) of the 74 most N-terminal amino acids of the SPB.

The amino acid sequence of an SPB transposase comprising an N-terminal nuclear localization sequence (NLS; SEQ ID NO: 1) was used to construct a SPB transposase sequence comprising an additional C-terminal CRD and a 74 amino acid NTD. Amino acid residues 542-594 of the SPB transposase (SEQ ID NO: 2), which comprises the SPB CRD domain, were joined to the C-terminus of the SPB transposase sequence using an AGGG linker to generate a SPB Dual CRD transposase comprising an N-terminal NLS. The integration and excision activities of the SPB transposase comprising dual CRDs will be compared to wild type SPB transposase using wild type and symmetrical inverted terminal repeat sequences (ITRs) as set forth in Example 3.

### Example 6: Identification of PiggyBac Thermostability Mutations

Computational algorithms were employed to generate a list of point mutations with the potential to improve protein thermostability of the PiggyBac transposase. In one instance, the chain C of the Piggybac cryoEM structure 6×67 was used as input for the algorithm FireProt2.0 (https://loschmidt.chemi.muni.cz/fireprotweb/). As output, the amino acid at each position predicted to result in the highest thermostability was calculated. Positions in which the prediction for the most thermostable amino acid differed from the wild type sequence are listed in Table 10:

TABLE 10
Predicted Thermostability Mutations

| | | |
|---|---|---|
| I182L | S301A | C420M |
| M185L | R315K | D421H |
| F200W | Q318G | N427D |
| V207I | E331R | Q434E |
| M226F | V336I | V436I |
| I231T | S373K | I474L |
| V240K | V381E | K500R |
| Q254N | T392S | S513P |
| A263E | A411N | K525P |
| S289A | S419T | R567D |
| M298L | | |

Each point mutation was cloned individually into a TAL-PBx transposase targeting LINE1 repetitive element LINE1 R2.2 TAL-PBx (SEQ ID NO: 16) comprising a +73 TAL C-terminus and an N-terminally deleted (delta 1-85) to generate SEQ ID NOs: 99-129.

An “all-in-one site-specific excision/integration episomal reporter” system was constructed to test the new mutants' ability to catalyze site-specific transposition. This episomal reporter system comprises a plasmid containing a piggyBac transposon donor along with a transposon integration site all on the same plasmid. The transposon in this plasmid disrupts the open reading frame of a GFP preceded by an EF1a promoter and followed by poly-adenylation signal sequence. The vector also contains, in the opposite orientation, a polyA and transcription pause site, a TTAA integration site adjacent to LINE1 R2.2 right target sequences (SEQ ID NO: 130) and 13 bp spacers, followed by a PEST destabilized mScarlet reporter gene and a poly-adenylation signal sequence. This “all-in-one site-specific excision/integration episomal reporter” (SEQ ID NO: 131), when transfected into cells alone, expresses no GFP, and little to no mScarlet. Upon transposon excision catalyzed by SPB, PBx, or ssSPB, the GFP coding sequence is restored and GFP is expressed. Upon site-specific integration of the CMV promoter containing transposon into its target site upstream of mScarlet gene, mScarlet is expressed at above background levels. The reporter design is described in more detail in International Patent Application Publication No. WO 2023/060089.

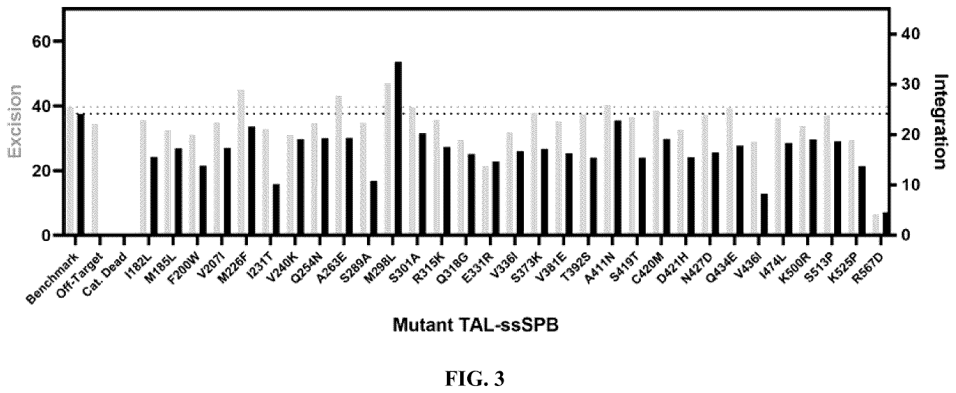

Each of the TAL-PBx SSM mutant expression vectors were co-transfected into HEK293T along with the all-in-one site-specific excision/integration episomal reporter. Briefly, a transfection mix containing 50 ng of a mutant TAL-PBx, 50 ng of the reporter plasmid, 0.3 μl of Transit2020 transfection reagent, in a total volume of 20p of serum free OptiMEM medium was assembled. To this, approximately 60,000 HEK293T cells in 180 μl of DMEM medium supplemented with 10% FBS were added, then 80 μl of this transfection mixture was plated in duplicate in clear bottom 96 well plates and incubated at 37° C. at 5% CO2. As controls, the benchmark TAL-ssSPB (SEQ ID NO: 16) or a TAL-ssSPB not specific for the LINE1 target, or a catalytically dead transposase were transfected in place of the mutant TAL-ssSPBs. GFP and mScarlet fluorescence were detected using an Incucyte live cell analysis instrument. The percent fluorescent cells for each of the excision (GFP) and site-specific integration (mScarlet) reporters is displayed in FIG. 3 and Table 11.

TABLE 11

| Mutant | Excision | Integration | Mutant | Excision | Integration |
|---|---|---|---|---|---|
| Benchmark | 39.71 | 24.15 | E331R | 21.53 | 14.7 |
| Off-Target | 34.56 | 0.29 | V336I | 31.91 | 16.74 |
| Cat. Dead | 0 | 0.1 | S373K | 38.01 | 17.15 |
| I182L | 35.75 | 15.59 | V381E | 35.38 | 16.33 |
| M185L | 32.71 | 17.25 | T392S | 37.47 | 15.43 |
| F200W | 31.22 | 13.8 | A411N | 40.48 | 22.85 |
| V207I | 35.09 | 17.37 | S419T | 36.6 | 15.43 |
| M226F | 45.14 | 21.6 | C420M | 38.65 | 19.18 |
| I231T | 32.99 | 10.14 | D421H | 32.83 | 15.52 |
| V240K | 31.16 | 19.16 | N427D | 37.33 | 16.48 |
| Q254N | 34.78 | 19.38 | Q434E | 39.26 | 17.85 |
| A263E | 43.38 | 19.39 | V436I | 29.09 | 8.25 |
| S289A | 34.97 | 10.82 | I474L | 36.45 | 18.35 |
| M298L | 47.16 | 34.51 | K500R | 33.92 | 19.11 |
| S301A | 39.72 | 20.25 | S513P | 37.14 | 18.71 |
| R315K | 35.83 | 17.61 | K525P | 29.66 | 13.75 |
| Q318G | 29.74 | 16.17 | R567D | 6.68 | 4.59 |

As shown in FIG. 3 and Table 11, the benchmark TAL-ssSPB resulted in excision activity and site-specific integration activity in approximately 40% and 24% of the cells, respectively. The off-target and catalytic dead controls resulted in no observed site-specific integration activity. The M298L mutant of TAL-ssSPB's excision and site-specific integration activity was higher than that of the benchmark TAL-ssSPB construct while all other mutants' integration activities were similar or lower than benchmark.

### Example 7: Construction and Evaluation of ssSPB with Hyperactive and Thermostability Mutations

In this example, the effect of combining hyperactive and thermostability mutations with dual CRD TAL-ssSPB was examined. LINE L2 TAL-ssSPB(d85+73) Dual CRD (SEQ ID NO: 17) and LINE R2.2 TAL-ssSPB(d85+73) Dual CRD (SEQ ID NO: 18) from Example 4 were modified to incorporate PBx hyperactive mutations into ssSPB. Specifically, the R372H mutation was introduced to each TAL-ssSPB to create SEQ ID NOs: 142 and 143, the S103P/S509G/N571S mutations were introduced to create SEQ ID NOs: 144 and 145, and the S103P/S509G/N571S/R372H mutations were introduced to create SEQ ID NOs: 146 and 147.

Each mutant pair of TAL-ssSPB were co-transfected into HEK293T cells with a transposon donor containing symmetrical ITRs and a PuroR-2A-GFP cargo (SEQ ID NO: 152). As controls, the mutant TAL-ssSPB− CRD transposases were replaced with the original wild type ssSPB, or by PBx. Briefly 120,000 HEK293T or HepG2 cells were plated in 24 well plates in 500 μL of DMEM+10% FBS one day before transfection. A 50 ng amount of the pair of transposase-expressing vectors was combined with 450 ng of the reporter and transfected using 1 μL of JetPrime transfection reagent. Two days after transfection, genomic DNA was harvested from the cells and site-specific integration of the transposon in the forward and reverse orientations at the LINE1 target was detected and quantified by ddPCR. The results are shown in Table 12.

TABLE 12

| | Forward | | Reverse | |
|---|---|---|---|---|
| | Integrations Per Haploid Genomes | | | |
| | in HEK293T Cells | | | |
| Benchmark | 2.38 | 2.58 | 2.36 | 2.68 |
| R372H | 3.29 | 2.47 | 3.49 | 2.59 |
| S103P/S509G/N571S | 3.15 | 2.57 | 3.15 | 2.68 |
| S103P/S509G/N571S/R372H | 3.38 | 4.23 | 3.62 | 4.47 |
| PBx | 0.002 | 0.004 | 0.003 | 0.004 |
| | Integrations Per Haploid Genomes | | | |
| | in HepG2 Cells | | | |
| Benchmark | 2.47 | 3.45 | 2.61 | 3.58 |
| R372H | 3.29 | 3.73 | 3.28 | 3.92 |
| S103P/S509G/N571S | 2.77 | 3.78 | 2.74 | 3.93 |
| S103P/S509G/N571S/R372H | 3.85 | 5.61 | 4.01 | 5.77 |
| PBx | 0.008 | 0.007 | 0.015 | 0.012 |

As shown in Table 12 increased site-specific integration was observed using the R372H mutant and the S103P/S509G/N571S mutant compared to the benchmark TAL-ssSPB. The highest site-specific integration was observed when all mutations were combined (S103P/S509G/N571S/R372H).

In another experiment, LINE L2 TAL-ssSPB(d85+73) Dual CRD (SEQ ID NO: 17) and LINE R2.2 TAL-ssSPB(d85+73) Dual CRD (SEQ ID NO: 18) from Example 4 were modified to incorporate PBx hyperactive and thermostability mutations into ssSPB. Specifically, the M298L mutation was introduced to each TAL-ssSPB to create SEQ ID NOs: 148 and 149, and the S103P/S509G/N571S/R372H/M298L mutations were introduced to create SEQ ID NOs: 150 and 151.

Site-specific integration activity of the Dual CRD TAL-ssSPB comprising five mutations (S103P/S509G/N571S/R372H/M298L) (SEQ ID NOs: 150 and 151) was compared to that of the Dual CRD TAL-ssSPB lacking S103P/S509G/N571S/R372H/M298L mutations (SEQ ID NOs: 17-18) and the single CRD TAL-ssSPB without S103P/S509G/N571S/R372H/M298L mutations (SEQ ID NOs: 15-16) in K562 cells and HEK293T cells. The integrations per haploid genome are shown as the sum of forward and reverse integrations in Table 13.

TABLE 13

| | Integrations Per Haploid Genomes | |
|---|---|---|
| | in K562 Cells | |
| Single CRD | 0.014 | 0.013 |
| Dual CRD | 0.249 | 0.302 |
| Dual CRD with five mutations | 0.413 | 0.392 |
| PBx | 0.000 | 0.000 |
| | Integrations Per Haploid Genomes | |
| | in HEK293T Cells | |
| Single CRD | Not tested | |
| Dual CRD | 4.51 | 4.67 |
| Dual CRD with five mutations | 9.74 | 11.02 |
| PBx | 0.029 | 0.000 |

As seen in Table 13, increased site-specific integrations were observed using the Dual CRD than with the single CRD TAL-ssSPB. The highest level of site-specific integration was seen using the Dual CRD comprising five mutations (S103P/S509G/N571S/R372H/M298L)

### Example 8: Construction and Evaluation of TAL-ssSPB with Shorter and Longer TAL Arrays

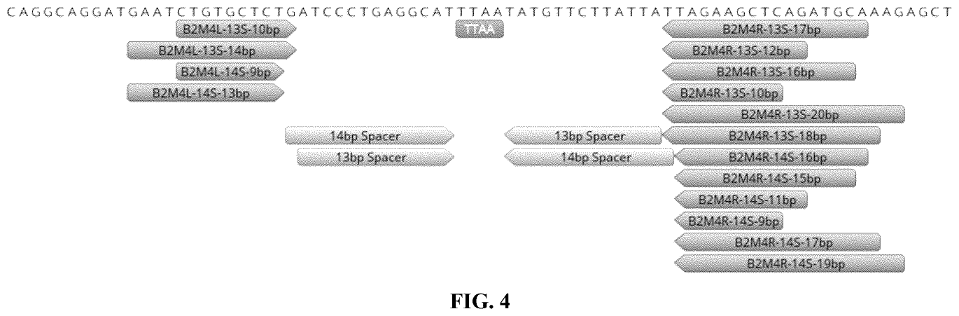

In this experiment, dual TAL-ssSPB constructs comprising five mutations (S103P/S509G/N571S/R372H/M298L) were assembled to target a TTAA within the human B2M gene at the sequence: CAGGCAGGATGAATCTGTGCTCTGATCCCTGAGGCATTTAATATGTTCTTATTATTAG AAGCTCAGATGCAAAGAGCT (SEQ ID NO: 153). Specifically, TAL arrays were designed to bind sequences spaced 13 bp or 14 bp from the TTAA integration site. Upstream of the TTAA, TAL arrays were designed to bind 9 bp, 10 bp, 13 bp, and 14 bp long targets. Downstream of the TTAA, TAL arrays were designed to bind 9 bp, 10 bp, 11 bp, 12 bp, 15 bp, 16 bp, 17 bp, 18 bp, 19 bp, and 20 bp long targets. The TAL target sequences, including the 5′T preceding the target, along with the TAL target length in bp, and spacing from the TTAA integration site in bp are listed in Table 14 and illustrated in FIG. 4.

TABLE 14

| | | |
|---|---|---|
| | TAL Target | |
| Target Sequence | Length | Spacing |

B2M Upstream (Left) Targets

| | | |
|---|---|---|
| TCTGTGCTCT (SEQ ID NO: 154) | 9 bp | 14 bp |
| TCTGTGCTCTG (SEQ ID NO: 155) | 10 bp | 13 bp |
| TGAATCTGTGCTCT (SEQ ID NO: 156) | 13 bp | 14 bp |
| TGAATCTGTGCTCTG (SEQ ID NO: 157) | 14 bp | 13 bp |

B2M Downstream (Right) Targets

| | | |
|---|---|---|
| TGAGCTTCTA (SEQ ID NO: 158) | 9 bp | 14 bp |
| TGAGCTTCTAA (SEQ ID NO: 159) | 10 bp | 13 bp |
| TCTGAGCTTCTA (SEQ ID NO: 160) | 11 bp | 14 bp |
| TCTGAGCTTCTAA (SEQ ID NO: 161) | 12 bp | 13 bp |
| TGCATCTGAGCTTCTA (SEQ ID NO: 162) | 15 bp | 14 bp |
| TGCATCTGAGCTTCTAA (SEQ ID NO: 163) | 16 bp | 13 bp |
| TTGCATCTGAGCTTCTA (SEQ ID NO: 164) | 16 bp | 14 bp |
| TTGCATCTGAGCTTCTAA (SEQ ID NO: 165) | 17 bp | 13 bp |
| TTTGCATCTGAGCTTCTA (SEQ ID NO: 166) | 17 bp | 14 bp |
| TTTGCATCTGAGCTTCTAA (SEQ ID NO: 167) | 18 bp | 13 bp |
| TCTTTGCATCTGAGCTTCTA (SEQ ID NO: 168) | 19 bp | 14 bp |
| TCTTTGCATCTGAGCTTCTAA (SEQ ID NO: 169) | 20 bp | 13 bp |

The TAL-ssSPBs were co-transfected into HEK293T cells as a pair (one upstream and one downstream) with a transposon donor containing symmetrical ITRs. Briefly 120,000 HEK293T cells were plated in 24 well plates in 500 μL of DMEM+10% FBS one day before transfection. A 50 ng amount of the pair of transposase-expressing vectors was combined with 450 ng of the reporter and transfected using 1 μL of JetPrime transfection reagent. In one experiment, either the 9 bp or 10 bp upstream TAL-ssSPB were co-transfected with each of the downstream TAL-ssSPBs. In another experiment either the 13 bp or 14 bp upstream TAL-ssSPB were co-transfected with each of the 11 bp-20 bp downstream TAL-ssSPBs. As a negative control, the TAL-ssSPB was replaced with PBx. Two days after transfection, genomic DNA was harvested from the cells and site-specific integration of the transposon in one orientation at the LINE1 target was detected and quantified by ddPCR. The results are shown in Table 15 and Table 16.

TABLE 15

| Upstream TAL-ssSPB | | | Downstream TAL-ssSPB | | | |
|---|---|---|---|---|---|---|
| TAL Length | | Spacing | TAL Length | Spacing | % B2M Alleles Edited | |
| 9 | bp | 14 bp | 9 bp | 14 bp | 3.42 | 3.06 |
| 9 | bp | 14 bp | 10 bp | 13 bp | 5.38 | 4.54 |
| 9 | bp | 14 bp | 11 bp | 14 bp | 4.07 | 4.17 |
| 9 | bp | 14 bp | 12 bp | 13 bp | 4.52 | 5.91 |
| 9 | bp | 14 bp | 15 bp | 14 bp | 4.50 | 4.31 |
| 9 | bp | 14 bp | 16 bp | 13 bp | 4.66 | 4.72 |
| 9 | bp | 14 bp | 16 bp | 14 bp | 4.62 | 5.02 |
| 9 | bp | 14 bp | 17 bp | 13 bp | 4.05 | 3.84 |
| 9 | bp | 14 bp | 17 bp | 14 bp | 4.68 | 4.67 |
| 9 | bp | 14 bp | 18 bp | 13 bp | 2.74 | 2.75 |
| 9 | bp | 14 bp | 19 bp | 14 bp | 4.39 | 3.82 |
| 9 | bp | 14 bp | 20 bp | 13 bp | 2.65 | 2.54 |
| 10 | bp | 13 bp | 9 bp | 14 bp | 5.73 | 5.72 |
| 10 | bp | 13 bp | 10 bp | 13 bp | 3.60 | 4.13 |
| 10 | bp | 13 bp | 11 bp | 14 bp | 4.62 | 5.76 |
| 10 | bp | 13 bp | 12 bp | 13 bp | 6.02 | 5.48 |
| 10 | bp | 13 bp | 15 bp | 14 bp | 4.12 | 5.39 |
| 10 | bp | 13 bp | 16 bp | 13 bp | 3.91 | 4.40 |
| 10 | bp | 13 bp | 16 bp | 14 bp | 4.54 | 6.22 |
| 10 | bp | 13 bp | 17 bp | 13 bp | 2.70 | 2.47 |
| 10 | bp | 13 bp | 17 bp | 14 bp | 4.30 | 4.12 |
| 10 | bp | 13 bp | 18 bp | 13 bp | 2.69 | 3.72 |
| 10 | bp | 13 bp | 19 bp | 14 bp | 2.64 | 3.37 |
| 10 | bp | 13 bp | 20 bp | 13 bp | 2.67 | 3.43 |

| | | | | | |
|---|---|---|---|---|---|
| PBx | PBx | PBx | PBx | 0.03 | 0.00 |

TABLE 16

| Upstream TAL-ssSPB | | Downstream TAL-ssSPB | | | |
|---|---|---|---|---|---|
| TAL Length | Spacing | TAL Length | Spacing | % B2M Alleles Edited | |
| 13 bp | 14 bp | 11 bp | 14 bp | 6.73 | 7.02 |
| 13 bp | 14 bp | 12 bp | 13 bp | 6.55 | 8.38 |
| 13 bp | 14 bp | 15 bp | 14 bp | 5.69 | 5.58 |
| 13 bp | 14 bp | 16 bp | 13 bp | 5.59 | 5.42 |
| 13 bp | 14 bp | 16 bp | 14 bp | 5.13 | 6.98 |
| 13 bp | 14 bp | 17 bp | 13 bp | 5.94 | 6.50 |
| 13 bp | 14 bp | 17 bp | 14 bp | 5.87 | 4.46 |
| 13 bp | 14 bp | 18 bp | 13 bp | 4.71 | 6.63 |
| 13 bp | 14 bp | 19 bp | 14 bp | 3.12 | 3.80 |
| 13 bp | 14 bp | 20 bp | 13 bp | 4.86 | 5.15 |
| 14 bp | 13 bp | 11 bp | 14 bp | 7.63 | 8.87 |
| 14 bp | 13 bp | 12 bp | 13 bp | 6.80 | 7.46 |
| 14 bp | 13 bp | 15 bp | 14 bp | 6.25 | 4.79 |
| 14 bp | 13 bp | 16 bp | 13 bp | 6.00 | 7.18 |
| 14 bp | 13 bp | 16 bp | 14 bp | 6.39 | 7.76 |
| 14 bp | 13 bp | 17 bp | 13 bp | 5.66 | 4.52 |
| 14 bp | 13 bp | 17 bp | 14 bp | 4.96 | 5.14 |
| 14 bp | 13 bp | 18 bp | 13 bp | 5.76 | 7.49 |
| 14 bp | 13 bp | 19 bp | 14 bp | 5.45 | 6.05 |
| 14 bp | 13 bp | 20 bp | 13 bp | 4.65 | 5.61 |
| PBx | PBx | PBx | PBx | 0.03 | 0.08 |

As shown in Table 15 and Table 16, the TAL-ssSPB pairs made comprising TAL arrays of varying lengths all catalyze site specific integration into the B2M genomic site and levels higher than the PBx control.

### Example 9: In Vivo Editing of Mouse Liver by TAL-ssSPB with a Dual CRD and Hyperactive and Thermostable Mutations

In this example, TAL-ssSPB with a dual CRD and hyperactive and thermostability mutations was used to edit the LINE1 locus in vivo in mouse liver cells. A TTAA within open reading frame 1 of the L1Mda family of mouse LINE1 sequences was selected as a target site. 10 bp long TAL binding sites were identified on each side of the TTAA separated from the TTAA by 13 bp spacers. The target site, with TAL binding sites underlined and TTAA in bold, is:

(SEQ ID NO: 175)
TCAAGAAGGACTTTCATAAGTCACTTAAAGATTTACAGGAGAGCACTGC
TAA.

A TAL array (SEQ ID NO: 170) to bind the left site (CAAGAAGGAC) and TAL array (SEQ ID NO: 171) to bind the reverse complement of the right site (TAGCAGTGCT) were designed. The TAL arrays were assembled into TAL-ssSPB expression constructs with dual CRD and hyperactive and thermostability mutations (S103P/S509G/N571S/R372H/M298L) as described above to generate mLINE1 left TAL-ssSPB (SEQ ID NO: 172) and mLINE1 right TAL-ssSPB (SEQ ID NO: 173). As an in vivo transposon donor (SEQ ID NO: 174), a nanoplasmid was assembled consisting of a promoter driving expression of a firefly luciferase gene. The firefly luciferase gene was interrupted by a piggybac transposon harboring symmetric ITRs and binding sites for PCR primer such that in its initial state a functional luciferase is not expressed. Upon transposase catalyzed excision of the transposon and seamless repair of the backbone, the luciferase gene is repaired leading to luciferase expression, quantifiable through luminescence readings. Site-specific integration of the excised transposon can then be detected and quantified using PCR approaches.

To perform in vivo site-specific transposition, 25 ug of plasmid DNA was delivered by hydrodynamic delivery (HDD) to female Balb/C mice aged between 10 and 12 weeks. Briefly, the HDD injection consisted of 25 ug of plasmid DNA in a 2 mL volume Trans-ITEE buffer (Mirus Bio) injected into the lateral tail vein over 3-5 seconds. The 25 ug dose of plasmid DNA consisted of 12.5 ug of the in vivo transposon donor DNA, 7.5 ug of the left mLINE1 TAL-ssSPB expression construct, and 7.5 ug of the right mLINE1 TAL-ssSPB expression construct. As negative controls, other groups of mice were injected with buffer only (PBS) or with 12.5 ug of the transposon donor DNA and 12.5 ug of an expression vector of an excision only mutant of piggyBac (PBx). Three days post-delivery, in vivo bioluminescent imaging (BLI) was performed to quantify the amount of transposon excision from the donor DNA catalyzed by the transposases (TAL-ssSPB or PBx). The mice were injected with 50 ul of IVISBrite D-Luciferin (Revvity Health Sciences) and luciferase signal was measured 6 minutes later as total flux in photons/second using IVIS imager (Perkin Elmer) with auto exposure time. Following BLI, the mice were sacrificed, and the left lobe of the liver was harvested. Genomic DNA was extracted from the harvested liver tissue for quantification of site-specific integration into the mouse LINE1 loci by digital droplet PCR (ddPCR). PCR amplicons generated from a primer within the transposon and a primer flanking the insertion site of the LINE1 genomic DNA target measured site-specific integration. A second PCR amplicon generated from two primers within an un-targeted genomic region was used to quantify total genomic DNA input into the PCR reaction. The site-specific integration frequencies, displayed as integrations per 100 haploid genomes, along with BLI measurements are shown in table 17. As seen in table 17, transposon excision was detected at similar levels in the PBx and TAL-ssSPB groups. As expected, site-specific integration of the transposon into the mouse LINE1 target site was only detected in the TAL-ssSPB group.

TABLE 17

| | | BLI Flux | Integrations Per 100 Haploid |
|---|---|---|---|
| Group | Mouse # | (photons/sec) | Genomes |
| PBS | 1 | 1.18E+04 | 0.000 |
| | 2 | 5.84E+04 | 0.000 |
| PBx | 1 | 1.09E+10 | 0.000 |
| | 2 | 4.08E+10 | 0.000 |
| TAL- | 1 | 7.51E+09 | 0.116 |
| ssSPB | 2 | 5.71E+10 | 1.318 |
| | 3 | 5.85E+10 | 1.351 |
| | 4 | 2.76E+10 | 0.914 |
| | 5 | 6.50E+10 | 0.527 |

What is claimed is:

1. A fusion protein comprising, in N-terminal to C-terminal order: a nuclear localization signal (NLS), a DNA targeting domain, and a first transposase domain comprising an amino acid sequence of any of SEQ ID NOs: 28-69.

2. The fusion protein of claim 1, wherein the first transposase domain comprises an amino acid sequence of any of SEQ ID NOs: 28-48.

3. The fusion protein of claim 1, wherein the first transposase domain comprises an amino acid sequence of any of SEQ ID NOs: 49-69.

4. The fusion protein of claim 2, wherein the first transposase domain comprises an amino acid sequence of SEQ ID NO: 30 or 38.

5. The fusion protein of claim 3, wherein the first transposase domain comprises an amino acid sequence of SEQ ID NO: 51 or 59.

6. The fusion protein of any of claims 1-5, wherein the DNA targeting domain comprises one or more Zinc Finger Motifs.

7. The fusion protein of claim 1 or 2, wherein the DNA targeting domain comprises the sequence of SEQ ID NO: 74.

8. The fusion protein of any of claims 1-5, wherein the DNA targeting domain comprises one or more TAL domains.

9. The fusion protein of claim 8, wherein the DNA targeting domain binds to a nucleic acid sequence encoding GFP, a LINE1 repeat element, or the zinc finger 268 (ZFM268) binding site.

10. A fusion protein, comprising:
(a) a TAL Array; and
(b) a modified Super piggyBac transposase (“SPB”) comprising a N-terminal deletion and a second cysteine rich domain (CRD) fused to the C-terminus of the SPB transposase;
wherein the C-terminus of the TAL Array is fused to the N-terminal amino acid of the N-terminal deleted SPB to generate a TAL Array-N-terminal deleted SPB fusion protein.

11. The fusion protein of claim 10, further comprising an GS or GGGS linker positioned between the TAL Array and the N-terminal deleted SPB.

12. The fusion protein of claim 10 or 11, wherein the SPB comprises a N-terminal deletion comprising a deletion of amino acids 1-83, 1-84, 1-85, 186, 1-87, 1-88, 1-89, 1-90, 1-91, 1-92, 1-93, 1-94, 1-95, 1-96, 1-97, 1-98, 1-99, 1-100, 1-101, 1-102 or 1-103.

13. A fusion protein, comprising:
(a) a TAL Array; and
(b) a modified Super piggyBac transposase (“SPB”) comprising a N-terminal deletion, one or more integration-deficient PBx mutations and a second cysteine rich domain (CRD) fused to the C-terminus of the SPB transposase;
wherein the C-terminus of the TAL Array is fused to the N-terminal amino acid of the N-terminal deleted SPB to generate a TAL Array-N-terminal deleted SPB fusion protein.

14. The fusion protein of claim 13, further comprising a GS or a GGGS linker positioned between the TAL Array and the N-terminal deleted SPB.

15. The fusion protein of claim 13 or 14, wherein the modified Super piggyBac transposase comprises the amino acid sequence of any of SEQ ID Nos: 28-69.

16. A fusion protein comprising, in N-terminal to C-terminal order: a DNA targeting domain and a first transposase domain comprising the sequence set forth in SEQ ID NO: 1 or 3, wherein the first transposase domain comprises a deletion of the 83-103 most N-terminal amino acids of SEQ ID NO: 1 or 73.

17. The fusion protein of claim 16, wherein the DNA targeting domain comprises one or more Zinc Finger Motifs.

18. The fusion protein of claim 16, wherein the DNA targeting domain comprises one or more TAL domains.

19. The fusion protein of any one of claims 16-18, wherein the DNA targeting domain binds to a nucleic acid sequence encoding GFP, zinc finger 268 (ZFM268), phenylalanine hydroxylase (PAH), beta-2-microglobulin (B2M) or a LINE1 repeat element.

20. The fusion protein of any one of claims 16-19, wherein the first transposase domain and the DNA targeting domain are connected by a linker.

21. The fusion protein of claim 20, wherein the linker comprises the sequence GGGGS.

22. The fusion protein of any one of claims 16-21, wherein the first transposase domain comprises an N-terminal deletion of amino acids 1-83, 1-84, 1-85, 186, 1-87, 1-88, 1-89, 1-90, 1-91, 1-92, 1-93, 1-94, 1-95, 1-96, 1-97, 1-98, 1-99, 1-100, 1-101, 1-102 or 1-103.

23. The fusion protein of any one of claims 16-22, wherein the first transposase domain comprises (a) at least one mutation selected from the group consisting of M185R, M185K, D197K, D197R, D198K, D198R, D201K, and D201R; or (b) at least one mutation selected from the group consisting of L204D, L204E, K500D, K500E, R504E, and R504D relative to SEQ ID NO: 1 or 73, with numbering beginning at the 12^{th} residue of SEQ ID NO: 1 and the first residue of SEQ ID NO: 73.

24. The fusion protein of any one of claim 16-23, further comprising a second transposase domain C-terminal to the first transposase domain, wherein the second transposase domain comprises the sequence set forth in SEQ ID NO: 1 or 73.

25. The fusion protein of claim 24, wherein the second transposase domain comprises a deletion of N-terminal amino acids 1-83, 1-84, 1-85, 186, 1-87, 1-88, 1-89, 1-90, 1-91, 1-92, 1-93, 1-94, 1-95, 1-96, 1-97, 1-98, 1-99, 1-100, 1-101, 1-102 or 1-103 of SEQ ID NO: 1 or 73.

26. The fusion protein of any one of claims 16-25, wherein the second transposase domain comprises (a) at least one mutation selected from the group consisting of M185R, M185K, D197K, D197R, D198K, D198R, D201K, and D201R; or (b) at least one mutation selected from the group consisting of L204D, L204E, K500D, K500E, R504E, and R504D relative to SEQ ID NO: 1 or 73 with numbering beginning at the 12^{th} residue of SEQ ID NO: 1 and the first residue of SEQ ID NO: 73.

27. A polynucleotide comprising a nucleic acid sequence encoding the fusion proteins of any one of claims 1-26.

28. A transposon, comprising symmetrical left end (LE) and right end (RE) inverted terminal repeat sequences (ITRs), wherein the nucleotide sequences of the LE ITR and the RE ITR are each SEQ ID NO: 6.

29. A transposon, comprising symmetrical left end (LE) and right end (RE) inverted terminal repeat sequences (ITRs), wherein the nucleotide sequence of the LE ITR comprises SEQ ID NO: 6 and the nucleotide sequence of the RE ITR comprises SEQ ID NO: 7, SEQ ID NO: 8, SEQ ID NO: 9, SEQ ID NO: 10, SEQ ID NO: 21, or SEQ ID NO: 98.

30. The transposon of claim 28 or 29, wherein the transposon comprises a nucleotide sequence encoding a therapeutic protein.

31. The transposon of claim 30, wherein the transposon comprises a promoter sequence controlling expression of the therapeutic protein.

32. A method for site specific integration of a therapeutic gene into one or more genomic locus of a cell, comprising co-introducing into the cell the transposon of any one of claims 28-30 and the polynucleotide of claim 27.

33. A method of modifying the genome of a cell, the method comprising: providing the cell with the fusion protein of any one of claims 1-26, wherein the cell comprises a modified binding site comprising, in 5′ to 3′ order, the reverse of the sequence of a target site for the DNA targeting domain, a first spacer, a TTAA target integration site for SPB, a second spacer, and the complement of the sequence of the target site for the DNA targeting domain.

34. A fusion protein comprising, in N-terminal to C-terminal order: a nuclear localization signal (NLS), a DNA targeting domain, and a first transposase domain comprising an amino acid sequence of any of SEQ ID NOs: 132-141.

35. The fusion protein of claim 34, wherein the DNA targeting domain comprises one or more TAL domains.

36. The fusion protein of claim 35, wherein the DNA targeting domain binds to a nucleic acid sequence encoding GFP, a LINE1 repeat element, or the zinc finger 268 (ZFM268) binding site.

37. The fusion protein of claim 35 or 36, wherein the fusion protein comprises the sequence set forth in SEQ ID NO: 142-151.
